(19) United States
(12) Patent Application Publication
(10) Pub. No.: US 2020/0323905 A1
(43) Pub. Date: Oct. 15, 2020

(54) METHODS AND COMPOSITIONS FOR MODULATING THE IMMUNE SYSTEM

(71) Applicants: Trustees of Boston University, Boston, MA (US); University of Maryland Baltimore County, Baltimore, MD (US)

(72) Inventors: Xaralabos VARELAS, Boston, MA (US); Eleni STAMPOULOGLOU, Boston, MA (US); Gregory SZETO, Baltimore, MD (US)

(73) Assignees: Trustees of Boston University, Boston, MA (US); University of Maryland Baltimore County, Baltimore, MD (US)

(21) Appl. No.: 16/849,591

(22) Filed: Apr. 15, 2020

Related U.S. Application Data
(60) Provisional application No. 62/833,877, filed on Apr. 15, 2019.
(60) Provisional application No. 62/950,664, filed on Dec. 19, 2019.

(51) Int. Cl. A61K 35/17

(52) U.S. Cl. CPC A61K 35/17; A61K 45/06

(57) ABSTRACT

Described herein are methods for activating or differentiating a T cell comprising contacting a T cell with a pharmaceutically effective amount of an agent that inhibits Yap and culturing the T cells for a time, and under conditions sufficient to induce activation or differentiation. Compositions comprising the activated or differentiated T cells, and methods for treating a disease or disorder caused by or associated with T cell dysfunction comprising administering the same are described herein. In one embodiment, the disease or disorder is cancer, an autoimmune disease or a microbial infectious disease.

### CROSS-REFERENCE TO RELATED APPLICATION

This application claims the benefit under 35 U.S.C. § 119(e) of U.S. Provisional Application Nos 62/833,877 filed on Apr. 15, 2019, and 62/950,664 filed on Dec. 19, 2019, the contents of which are incorporated herein by reference in their entireties.

### GOVERNMENT SUPPORT

This invention was made with Government support under Grant Nos R01HL124392 awarded by the National Institutes of Health. The Government has certain rights in the invention.

### FIELD OF THE INVENTION

The field of the invention relates to methods for modulating (e.g., activating, deactivating, differentiating, etc.) T cells and uses thereof.

### SEQUENCE LISTING

The instant application contains a Sequence Listing which has been submitted electronically in ASCII format and is hereby incorporated by reference in its entirety. Said ASCII copy, created on Apr. 27, 2020, is named 701586-095060WOPT_SL.txt and is 17,353 bytes in size.

### BACKGROUND

The immune system is a collection of organs, cells and specialized tissues that work together to defend the body against foreign invaders and diseased cells. A healthy immune system can recognize foreign or aberrant cells and target them for destruction. However, disorders such as cancer, autoimmune disease, and infectious disease can wreak havoc.

According to the World Health Organization, an estimated 9.6 million deaths globally were attributed to cancer in 2018. Moreover, statistics indicate that the cancer incidence rate is on the rise around the globe. In America, for example, projections suggest that fifty percent of those alive today will be diagnosed with some form of cancer at some point in their lives.

Recent outbreaks of emerging and re-emerging infectious diseases worldwide, such as COVID-19, severe acute respiratory syndrome (SARS), Middle East respiratory syndrome (MERS), measles, avian, pandemic influenza, chikungunya virus, Ebola virus disease (EVD), Zika virus disease, have resulted in a renewed focus on infectious diseases. There is a need for constant readiness and preparedness to deal with infectious disease outbreaks including emerging and re-emerging infectious disease threats.

The dynamic relationship between the immune system and disease development has been well described over the past few decades. Cluster of differentiation 8-positive (CD8⁺) and cluster of differentiation 4-positive (CD4⁺) T cells are central players in the adaptive immune system. T cells elicit targeted, antigen-specific responses for direct killing of an infected or transformed cell, shaping and regulating the immune response in host defense. Most mature T cells circulate in a resting, naïve state, and upon cognate antigen recognition, T cells become activated, proliferate clonally, and differentiate into effector T cells. Naïve CD8⁺ T cells differentiate into cytotoxic T cells, while CD4⁺ T cells differentiate into an array of different types of helper (i.e., T helper cell type 1 [Th1], Th2, Th17) or regulatory T cells (Tregs) depending on microenvironmental cues. Each phenotype is defined by expression of signature transcription factors and effector cytokines leading to distinct functions. T-cell activation also up-regulates negative feedback mechanisms, such as inhibitory receptors, which minimize pathogenic inflammation and autoimmunity. This network of immunosuppressive factors is frequently co-opted in infections and cancer, leading to terminally differentiated and exhausted T cells that lose effector function and ability to infiltrate disease sites.

The finding that revitalization of exhausted, dysfunctional T cells can restore the immune response has revolutionized cancer therapy with the use of checkpoint inhibition. Chimeric antigen receptor T cells (CAR-Ts), engineered for enhanced antigen recognition and costimulation, also demonstrate promising clinical efficacy. However, both immunotherapies are effective for only a fraction of patients. Major challenges to extending the efficacy of immunotherapy to more cancer patients include sustaining T-cell activation and achieving T-cell infiltration in the immunosuppressive microenvironment of solid tumors.

Past research has implicated the Hippo pathway, the Yes-associated protein (Yap) and the transcriptional coactivator with PDZ-binding motif (Taz) in T cell development, however the role of YAP or TAZ in regulating T cell effector functions is vague.

Accordingly, there is a need in the art of immunotherapy to uncover novel methods and pharmaceutical compositions to enhance T cell activation, differentiation, migration and cytotoxicity.

### SUMMARY

The present invention described herein is based, in part, on the discovery that inhibition of YAP activity enhances immune responses and leads to tumor growth inhibition and increased memory T cell generation by increasing T cell activation, migration and cytotoxicity and modulating differentiation. Inhibition of Yap activity further improves the success rate of immunotherapies. YAP functions as a novel immunomodulatory factor in T cells, and that inhibition of YAP activity enhances CD4+ and CD8+ T cell activation, differentiation and function in vitro and in vivo.

As shown herein, Yap levels are elevated upon T-cell activation and conditional deletion of the Yap gene in CD4+ and CD8+ T cells enhanced their activation and differentiation potential. Accordingly, one aspect provided herein is a method for modulating, for example, activating a T cell, the method comprising contacting a T cell with a pharmaceutically effective amount of an agent that inhibits Yap and culturing the T cells for a time, and under conditions sufficient to induce activation. In another embodiment, one can add Yap or enhance expression of Yap to deactivate activated T cells.

Another aspect provided herein is a method for differentiating a T cell, the method comprising contacting a T cell with a pharmaceutically effective amount of an agent that inhibits Yap and culturing the T cells for a time, and under conditions sufficient to induce differentiation.

In one embodiment of any aspect provided herein, the contacting is ex vivo, in vivo, or in vitro.

In one embodiment of any aspect provided herein, the culturing is ex vivo, in vivo, or in vitro.

In one embodiment of any aspect provided herein, the method further comprises the step of, prior to contacting, obtaining a T cell from a biological source prior.

In one embodiment of any aspect provided herein, the T cell is naïve T cell, a cytotoxic T cell, a memory T cell, a natural killer T cell, a tumor infiltrating T cell, a regulatory T cell, a helper T cell, or a synthetic T cell. In one embodiment of any aspect provided herein, the T cell is T cell is a αβ T cell, γδ T cell, CD8+ T cell, or a CD4+ T cell.

In one embodiment of any aspect provided herein, the biological source is a mammal.

In one embodiment of any aspect provided herein, the T cell is genetically modified prior to contacting. In one embodiment of any aspect provided herein, the T cell is engineered to comprise a chimeric antigen receptor prior to contacting.

In one embodiment of any aspect provided herein, the agent that inhibits YAP is selected from the group consisting of a small molecule, an antibody or antibody reagent, a protein, a peptide, a genome editing system, an antisense oligonucleotide, and an RNAi.

In one embodiment of any aspect provided herein, the small molecule is Verteporfin or YAP/TAZ inhibitor-1. In one embodiment, the agent that inhibits Yap is a statin. In one embodiment, the agent that inhibits YAP antagonist comprises simvastatin. In another aspect, exemplary YAP antagonists include β-adrenergic receptor agonists, Dobutamine, Latrunculin A, Latrunculin B, cytochalasin D, actin inhibitors, drugs that act on the cytoskeleton, Blebbistatitin, Botulinum toxin C3, and RHO kinase-targeting drugs (e.g., Y27632).

In one embodiment of any aspect provided herein, the RNAi is a microRNA, an siRNA, or a shRNA.

In one embodiment of any aspect provided herein, the gene editing system is CRISPR-Cas gene editing. In one embodiment, the CRISPR system is a null system. In one embodiment, the peptide or protein binds to nucleic acid of the Yap gene and inhibits it.

In one embodiment of any aspect provided herein, inhibiting YAP is inhibiting the expression level and/or activity of YAP in the T cell. In one embodiment of any aspect provided herein, the expression level and/or activity of YAP is inhibited by at least 50%, at least 60%, at least 70%, at least 80%, at least 90%, or more as compared to an appropriate control.

In one embodiment of any aspect provided herein, inhibiting the expression level and/or activity of YAP in the T cell increases the T cell's capacity to infiltrate a tumor microenvironment and/or tumor.

In one embodiment of any aspect provided herein, inhibiting the expression level and/or activity of YAP in the T cell increases expression of chemokine or chemokine receptor genes in the T cell. For example, those chemokines or chemokine receptors listed in Table 3 herein.

In one embodiment of any aspect provided herein, inhibiting the expression level and/or activity of YAP in the T cell increases expression of at least one of TEAD1, TEAD2, TEAD3 or TEAD4 protein in the T cell.

In one embodiment of any aspect provided herein, inhibiting the expression level and/or activity of YAP in the T cell increases expression of WW domain-containing transcription regulator protein 1 (WWTR1/Taz) in the T cell.

In one embodiment of any aspect provided herein, the method further comprises contacting the T cell with an agent that inhibits WWTR1/Taz.

In one embodiment of any aspect provided herein, inhibiting WWTR1/Taz is decreasing the expression level and/or activity of WWTR1/Taz in the T cell. In one embodiment of any aspect provided herein, the expression level and/or activity of WWTR/Taz is decreased by at least 50%, at least 60%, at least 70%, at least 80%, at least 90%, or more as compared to an appropriate control.

In one embodiment of any aspect provided herein, the method further comprises engineering the T cell to comprise a chimeric antigen receptor. In one embodiment of any aspect provided herein, the method further comprises genetically modifying the T cell.

In one embodiment of any aspect provided herein, the method further comprises, after culturing, transplanting said population of contacted T cells into a recipient subject.

In one embodiment of any aspect provided herein, the population of contacted T cells is autologous to the recipient subject. In one embodiment of any aspect provided herein, the population of contacted T cells is allogeneic to the recipient subject. In one embodiment of any aspect provided herein, the population of contacted T cells is xenogeneic to the recipient subject.

Another aspect provided herein is a method for activating or differentiating a T cell comprising (a) providing a population of T cells, (b) contacting the T cell population with an agent that inhibits Yap, and (c) ex vivo culturing for a time, and underconditions sufficient to induce activation of the T cell population.

Another aspect provided herein is an activated T cell produced any of the methods described herein.

Another aspect provided herein is a differentiated T cell produced by any of the methods described herein.

Another aspect provided herein is a composition comprising any of the activated T cells or differentiated T cells described herein.

In one embodiment of any aspect provided herein, the composition is formulated for T cell transplantation.

In one embodiment of any aspect provided herein, the composition further comprises a pharmaceutically acceptable carrier.

Another aspect provided herein is a pharmaceutical composition comprising an agent that inhibits YAP and a pharmaceutically acceptable carrier.

Another aspect provided herein is a pharmaceutical composition comprising an agent that inhibits YAP and an agent that inhibits WWTR1/Taz.

Another aspect provided herein is a method for T cell transplantation comprising (a) providing a population of T cells; (b) contacting the T cell population with an agent that inhibits Yap; (c) ex vivo culturing for a time, and under conditions sufficient to induce activation; and (d) transplanting said population of contacted T cells into a recipient subject.

In one embodiment of any aspect provided herein, the population of contacted T cells is provided to the recipient subject.

Another aspect provided herein is a method of treating or preventing a disease or disorder caused by or associated with T cell dysfunction, the method comprising administering to a subject in need thereof a therapeutically effective amount of any of the activated T cells or differentiated T cells described herein, any of the compositions described herein, or any agent that inhibits YAP described herein.

In one embodiment of any aspect provided herein, the disease or disorder caused by or associated with T cell dysfunction is selected from the group consisting of a cancer, an autoimmune disease, and a microbial infectious disease.

In one embodiment of any aspect provided herein, the cancer is a carcinoma, a sarcoma, a melanoma, a lymphoma, and a leukemia. In one embodiment of any aspect provided herein, the cancer is a melanoma or a lung cancer.

In one embodiment of any aspect provided herein, the microbial infectious disease is caused by a fungal, bacterial, or viral infection. In one embodiment of any aspect provided herein, the fungal, bacterial, or viral infection is systemic or local.

In one embodiment of any aspect provided herein, the autoimmune disease is selected from the groups consisting of Lupus, Type I Diabetes, Sjögren's syndrome, Rheumatoid arthritis, Inflammatory bowel disease, Multiple sclerosis, Psoriasis, Guillain-Barre syndrome, Chronic inflammatory demyelinating polyneuropathy, Graves' disease, Hashimoto's thyroiditis, Myasthenia gravis, Vasculitis, Addison's disease, Pernicious anemia, Celiac disease, Systemic lupus erythematosus, Cutaneous lupus erythematosus, and Aplastic anemia.

In one embodiment of any aspect provided herein, the T cell dysfunction is due to increased Yap expression in the T cell as compared to a reference level.

In one embodiment of any aspect provided herein, the agent further comprises a T cell targeting moiety. In one embodiment of any aspect provided herein, the T cell targeting moiety specifically binds to a T cell-specific cell-surface polypeptide. In one embodiment of any aspect provided herein, the T cell-specific cell surface polypeptide is CD4 or CD8. In one embodiment, one can use these agents to fine-tune and modulate CD4+ and/or CD8+ T cell responses.

In one embodiment of any aspect provided herein, the agent is formulated for delivery to a T cell.

In one embodiment of any aspect provided herein, the method further comprises administering an agent that inhibits WWTR/Taz.

In one embodiment of any aspect provided herein, the method further comprises administering at least a second therapeutic.

In one embodiment of any aspect provided herein, the second therapeutic is an anti-microbial therapeutic, an anti-autoimmune disease therapeutic, and an anti-cancer therapeutic.

Another aspect provided herein is a method of treating or preventing a disease or disorder caused by or associated with T cell dysfunction, the method comprising administering to a subject in need thereof a therapeutically effective amount of an agent that inhibits YAP and an agent that inhibits WWTR1/Taz.

Another aspect provided herein is a method of treating a microbial infectious disease, the method comprising administering to a subject in need thereof an anti-microbial therapeutic; and any of the activated T cells or differentiated T cells described herein, any of the compositions described herein, or any agent that inhibits YAP described herein.

Another aspect provided herein is a method of treating an autoimmune disease, the method comprising administering to a subject in need thereof an anti-autoimmune therapeutic; and any of the activated T cells or differentiated T cells described herein, any of the compositions described herein, or any agent that inhibits YAP described herein.

Another aspect provided herein is a method of treating a cancer, the method comprising administering to a subject in need thereof an anti-cancer therapeutic; and any of the activated T cells or differentiated T cells described herein, any of the compositions described herein, or any agent that inhibits YAP described herein.

Another aspect provided herein is a method of increasing the efficacy of a vaccine, the method comprising administering to a subject in need thereof a vaccine; and any of the activated T cells or differentiated T cells described herein, any of the compositions described herein, or any agent that inhibits YAP described herein.

In one embodiment of any aspect provided herein, the method further comprises, after culturing, assessing the level of Yap in the T cell.

### Definitions

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

Definitions of common terms in immunology and molecular biology can be found in The Merck Manual of Diagnosis and Therapy, 19th Edition, published by Merck Sharp & Dohme Corp., 2011 (ISBN 978-0-911910-19-3); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), Taylor & Francis Limited, 2014 (ISBN 0815345305, 9780815345305); Lewin's Genes XI, published by Jones & Bartlett Publishers, 2014 (ISBN-1449659055); Michael Richard Green and Joseph Sambrook, Molecular Cloning: A Laboratory Manual, 4^{th} ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g. the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. Where applicable, a decrease can be preferably down to a level accepted as within the range of normal for an individual without a given disorder.

The terms “increased”, “increase”, or “enhance” are all used herein to mean an increase by a statically significant amount. In some embodiments, the terms “increased”, “increase”, or “enhance” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, a 20 fold increase, a 30 fold increase, a 40 fold increase, a 50 fold increase, a 6 fold increase, a 75 fold increase, a 100 fold increase, etc. or any increase between 2-fold and 10-fold or greater as compared to a reference level. In the context of a marker or symptom, an “increase” is a statistically significant increase in such level.

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. In one embodiment, the subject is only a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of e.g., cancer. A subject can be male or female.

A subject can be one who has been previously diagnosed with or identified as suffering from or having a condition in need of treatment (e.g. a disease or disorder caused by or associated with T cell dysfunction) or one or more complications related to such a condition, and optionally, have already undergone treatment for the condition or the one or more complications related to the condition. Alternatively, a subject can also be one who has not been previously diagnosed as having such condition or related complications. For example, a subject can be one who exhibits one or more risk factors for the condition or one or more complications related to the condition or a subject who does not exhibit risk factors.

A “subject in need” of treatment for a particular condition can be a subject having that condition, diagnosed as having that condition, or at risk of developing that condition.

A “disease” is a state of health of an animal, for example a human, wherein the animal cannot maintain homeostasis, and wherein if the disease is not ameliorated, then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, “cancer” refers to a hyperproliferation of cells that have lost normal cellular control, resulting in unregulated growth, lack of differentiation, local tissue invasion, and metastasis. Cancers are classified based on the histological type (e.g., the tissue in which they originate) and their primary site (e.g., the location of the body the cancer first develops), and can be a carcinoma, a melanoma, a sarcoma, a myeloma, a leukemia, or a lymphoma. “Cancer” can also refer to a solid tumor. As used herein, the term “tumor” refers to an abnormal growth of cells or tissues, e.g., of malignant type or benign type. “Cancer” can be metastatic, meaning the cancer cells have disseminated from its primary site of origin and migrated to a secondary site.

An “autoimmune disease or disorder” is characterized by the inability of one's immune system to distinguish between a foreign cell and a healthy cell of one's own body. This results in one's immune system mounting a response to target one's healthy cells.

As used herein, “microbial infectious disease” refers to a disease state resulting from an infection comprising bacteria, virus, parasite and/or fungus. An infectious disease is characterized by its transmission, e.g., passing of a microbe causing the infectious disease from an infected host individual or group to a recipient individual or group, regardless of whether the recipient individual or group was previously infected. The presence can be abnormal in that the microbe is a noncommensal species, e.g. one not typically found in or on a healthy subject, it can be abnormal as in in a localization that the bacteria does not normally colonize (e.g., in the lungs, respiratory system, or sinus cavity), or it can be abnormal in that the microbe is present at abnormally high levels, e.g. at least twice the level found in or on a healthy subject (e.g. twice the level, three times the level, four times the level, five times the level, or greater), or it can be abnormal in that the presence of the microbe is causing or contributing to disease or symptoms thereof, e.g. necrosis, disfigurement, delayed wound healing, etc.

In some embodiments, “activation” can refer to the state of a T cell that has been sufficiently stimulated to induce detectable cellular proliferation. In some embodiments activation can refer to induced cytokine production. In other embodiments, activation can refer to detectable effector functions. At a minimum, an “activated T cells” as used herein is a proliferative T cell. In one embodiment, an activated T cell can be assessed by its cell-surface molecule profile. Non-limiting examples of molecules expressed the surface of an activated T cell include, but are not limited to CD44, CD69, CD71, CD25, and HLA-DR. Activated T cells also secrete cytokines, including, but not limited to IL-2. Methods to identify these surface molecules and secreted cytokines are known in the art.

In one embodiment, the term “engineered” and its grammatical equivalents as used herein can refer to one or more human-designed alterations of a nucleic acid, e.g., the nucleic acid within an organism's genome. In another embodiment, engineered can refer to alterations, additions, and/or deletion of genes. An “engineered cell” can refer to a cell with an added, deleted and/or altered gene. The term “cell” or “engineered cell” and their grammatical equivalents as used herein can refer to a cell of human or non-human animal origin.

In the various embodiments described herein, it is further contemplated that variants (naturally occurring or otherwise), alleles, homologs, conservatively modified variants, and/or conservative substitution variants of any of the particular polypeptides described are encompassed. As to amino acid sequences, one of ordinary skill will recognize that individual substitutions, deletions or additions to a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid and retains the desired activity of the polypeptide. Such conservatively modified variants are in addition to and do not exclude polymorphic variants, interspecies homologs, and alleles consistent with the disclosure.

A given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are well known. Polypeptides comprising conservative amino acid substitutions can be tested in any one of the assays described herein to confirm that a desired activity, e.g. CSR activity and specificity of a native or reference polypeptide is retained.

Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class. Particular conservative substitutions include, for example; Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

In some embodiments, the polypeptide described herein (or a nucleic acid encoding such a polypeptide) can be a functional fragment of one of the amino acid sequences described herein. As used herein, a “functional fragment” is a fragment or segment of a peptide which retains at least 50% of the wildtype reference polypeptide's activity according to an assay known in the art or described below herein. A functional fragment can comprise conservative substitutions of the sequences disclosed herein.

In some embodiments, the polypeptide described herein can be a variant of a sequence described herein. In some embodiments, the variant is a conservatively modified variant. Conservative substitution variants can be obtained by mutations of native nucleotide sequences, for example. A “variant,” as referred to herein, is a polypeptide substantially homologous to a native or reference polypeptide, but which has an amino acid sequence different from that of the native or reference polypeptide because of one or a plurality of deletions, insertions or substitutions. Variant polypeptide-encoding DNA sequences encompass sequences that comprise one or more additions, deletions, or substitutions of nucleotides when compared to a native or reference DNA sequence, but that encode a variant protein or fragment thereof that retains activity of the non-variant polypeptide. A wide variety of PCR-based site-specific mutagenesis approaches are known in the art and can be applied by the ordinarily skilled artisan.

A variant amino acid or DNA sequence can be at least 90%, at least 91%, at least 92%, at least 93%, at least 94%, at least 95%, at least 96%, at least 97%, at least 98%, at least 99%, or more, identical to a native or reference sequence. The degree of homology (percent identity) between a native and a mutant sequence can be determined, for example, by comparing the two sequences using freely available computer programs commonly employed for this purpose on the world wide web (e.g. BLASTp or BLASTn with default settings).

Alterations of the native amino acid sequence can be accomplished by any of a number of techniques known to one of skill in the art. Mutations can be introduced, for example, at particular loci by synthesizing oligonucleotides containing a mutant sequence, flanked by restriction sites permitting ligation to fragments of the native sequence. Following ligation, the resulting reconstructed sequence encodes an analog having the desired amino acid insertion, substitution, or deletion. Alternatively, oligonucleotide-directed site-specific mutagenesis procedures can be employed to provide an altered nucleotide sequence having particular codons altered according to the substitution, deletion, or insertion required. Techniques for making such alterations are very well established and include, for example, those disclosed by Walder et al. (Gene 42:133, 1986); Bauer et al. (Gene 37:73, 1985); Craik (BioTechniques, January 1985, 12-19); Smith et al. (Genetic Engineering: Principles and Methods, Plenum Press, 1981); and U.S. Pat. Nos. 4,518,584 and 4,737,462, which are herein incorporated by reference in their entireties. Any cysteine residue not involved in maintaining the proper conformation of the polypeptide also can be substituted, generally with serine, to improve the oxidative stability of the molecule and prevent aberrant crosslinking. Conversely, cysteine bond(s) can be added to the polypeptide to improve its stability or facilitate oligomerization.

As used herein, the term “DNA” is defined as deoxyribonucleic acid. The term “polynucleotide” is used herein interchangeably with “nucleic acid” to indicate a polymer of nucleosides. Typically, a polynucleotide is composed of nucleosides that are naturally found in DNA or RNA (e.g., adenosine, thymidine, guanosine, cytidine, uridine, deoxyadenosine, deoxythymidine, deoxyguanosine, and deoxycytidine) joined by phosphodiester bonds. However, the term encompasses molecules comprising nucleosides or nucleoside analogs containing chemically or biologically modified bases, modified backbones, etc., whether or not found in naturally occurring nucleic acids, and such molecules may be preferred for certain applications. Where this application refers to a polynucleotide it is understood that both DNA, RNA, and in each case both single- and double-stranded forms (and complements of each single-stranded molecule) are provided. “Polynucleotide sequence” as used herein can refer to the polynucleotide material itself and/or to the sequence information (i.e. the succession of letters used as abbreviations for bases) that biochemically characterizes a specific nucleic acid. A polynucleotide sequence presented herein is presented in a 5′ to 3′ direction unless otherwise indicated.

The term “polypeptide” as used herein refers to a polymer of amino acids. The terms “protein” and “polypeptide” are used interchangeably herein. A peptide is a relatively short polypeptide, typically between about 2 and 60 amino acids in length. Polypeptides used herein typically contain amino acids such as the 20 L-amino acids that are most commonly found in proteins. However, other amino acids and/or amino acid analogs known in the art can be used. One or more of the amino acids in a polypeptide may be modified, for example, by the addition of a chemical entity such as a carbohydrate group, a phosphate group, a fatty acid group, a linker for conjugation, functionalization, etc. A polypeptide that has a nonpolypeptide moiety covalently or noncovalently associated therewith is still considered a “polypeptide”. Exemplary modifications include glycosylation and palmitoylation. Polypeptides can be purified from natural sources, produced using recombinant DNA technology or synthesized through chemical means such as conventional solid phase peptide synthesis, etc. The term “polypeptide sequence” or “amino acid sequence” as used herein can refer to the polypeptide material itself and/or to the sequence information (i.e., the succession of letters or three letter codes used as abbreviations for amino acid names) that biochemically characterizes a polypeptide. A polypeptide sequence presented herein is presented in an N-terminal to C-terminal direction unless otherwise indicated.

In some embodiments, a nucleic acid encoding a polypeptide as described herein (e.g. a WWTR1/Taz polypeptide) is comprised by a vector. In some of the aspects described herein, a nucleic acid sequence encoding a given polypeptide as described herein, or any module thereof, is operably linked to a vector. The term “vector”, as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a plasmid, phage, transposon, cosmid, artificial chromosome, virus, virion, etc.

As used herein, the terms “treat,” “treatment,” or “treating,” refer to therapeutic treatments, wherein the object is to reverse, alleviate, ameliorate, inhibit, slow down or stop the progression or severity of a condition associated with a disease or disorder, e.g. a disease or disorder caused by or associated with T cell dysfunction. The term “treating” includes reducing or alleviating at least one adverse effect or symptom of a condition, disease or disorder. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

As used herein “preventing” or “prevention” refers to any methodology where the disease state or disorder (e.g., a disease or disorder caused by or associated with T cell dysfunction) does not occur due to the actions of the methodology (such as, for example, administration of any therapeutic treatments described herein). In one aspect, it is understood that prevention can also mean that the disease is not established to the extent that occurs in untreated controls. For example, there can be a 5, 10, 15, 20, 25, 30, 35, 40, 50, 60, 70, 80, 90, or 100% reduction in the establishment of disease frequency relative to untreated controls. Accordingly, prevention of a disease encompasses a reduction in the likelihood that a subject will develop the disease, relative to an untreated subject (e.g. a subject who is not administered a therapeutic described herein).

As used herein, the term “administering,” refers to the placement of a therapeutic or pharmaceutical composition as disclosed herein into a subject by a method or route which results in at least partial delivery of the agent at a desired site. Pharmaceutical compositions comprising agents as disclosed herein can be administered by any appropriate route which results in an effective treatment in the subject.

As used herein, an “agent” refers to e.g., a molecule, protein, peptide, antibody, or nucleic acid, that inhibits expression of a polypeptide or polynucleotide, or binds to, partially or totally blocks stimulation, decreases, prevents, delays activation, inactivates, desensitizes, or down regulates the activity of the polypeptide or the polynucleotide. Agents that inhibit Yap, e.g., inhibit expression, e.g., translation, post-translational processing, stability, degradation, or nuclear or cytoplasmic localization of a polypeptide, or transcription, post transcriptional processing, stability or degradation of a polynucleotide or bind to, partially or totally block stimulation, DNA binding, transcription factor activity or enzymatic activity, decrease, prevent, delay activation, inactivate, desensitize, or down regulate the activity of a polypeptide or polynucleotide. An agent can act directly or indirectly. In an alternative embodiment, the agent, e.g., that upmodulate Yap expression, can take an activated T cell and deactivate it. This would be useful in autoimmune diseases, dealing with transplants, etc.

The term “agent” as used herein means any compound or substance such as, but not limited to, a small molecule, nucleic acid, polypeptide, peptide, drug, ion, etc. An “agent” can be any chemical, entity or moiety, including without limitation synthetic and naturally-occurring proteinaceous and non-proteinaceous entities. In some embodiments, an agent is nucleic acid, nucleic acid analogues, proteins, antibodies, peptides, aptamers, oligomer of nucleic acids, amino acids, or carbohydrates including without limitation proteins, oligonucleotides, ribozymes, DNAzymes, glycoproteins, siRNAs, lipoproteins, aptamers, and modifications and combinations thereof etc. In certain embodiments, agents are small molecule having a chemical moiety. For example, chemical moieties included unsubstituted or substituted alkyl, aromatic, or heterocyclyl moieties including macrolides, leptomycins and related natural products or analogues thereof. Compounds can be known to have a desired activity and/or property, or can be selected from a library of diverse compounds.

The agent can be a molecule from one or more chemical classes, e.g., organic molecules, which may include organometallic molecules, inorganic molecules, genetic sequences, etc. Agents may also be fusion proteins from one or more proteins, chimeric proteins (for example domain switching or homologous recombination of functionally significant regions of related or different molecules), synthetic proteins or other protein variations including substitutions, deletions, insertion and other variants.

As used herein, the term “small molecule” refers to a chemical agent which can include, but is not limited to, a peptide, a peptidomimetic, an amino acid, an amino acid analog, a polynucleotide, a polynucleotide analog, an aptamer, a nucleotide, a nucleotide analog, an organic or inorganic compound (e.g., including heterorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

The term “RNAi” as used herein refers to interfering RNA or RNA interference. RNAi refers to a means of selective post-transcriptional gene silencing by destruction of specific mRNA by molecules that bind and inhibit the processing of mRNA, for example inhibit mRNA translation or result in mRNA degradation. As used herein, the term “RNAi” refers to any type of interfering RNA, including but are not limited to, siRNA, shRNA, endogenous microRNA and artificial microRNA. For instance, it includes sequences previously identified as siRNA, regardless of the mechanism of down-stream processing of the RNA (i.e. although siRNAs are believed to have a specific method of in vivo processing resulting in the cleavage of mRNA, such sequences can be incorporated into the vectors in the context of the flanking sequences described herein). Null CRISPR and other gene silencing methods can be used.

Methods and compositions described herein require that the levels and/or activity of Yap are inhibited. As used herein, Yes1 associated transcriptional regulator (Yap), also known as YAP, YKI, COB1, YAP2, and YAP65 refers to a downstream nuclear effector of the Hippo signaling involved in development, growth, repair, and homeostasis. This gene plays a known role in the development and progression of multiple cancers, as a transcriptional regulator of this signaling pathway and may function as a potential target for cancer treatment. Alternative splicing results in multiple transcript variants encoding different isoforms. Yap sequences are known for a number of species, e.g., human Yap, isoform 1 (NCBI Gene ID: 10413) polypeptide (e.g., NCBI Ref Seq NP_001123617.1) and mRNA (e.g., NCBI Ref Seq NM_001130145.3). Yap can refer to human Yap, including naturally occurring variants, isoforms, molecules, and alleles thereof. Yap refers to the mammalian Yap of, e.g., mouse, rat, rabbit, dog, cat, cow, horse, pig, and the like. The nucleic sequence of SEQ ID NO: 1 comprises a nucleic sequence which encodes Yap. The amino acid sequence of SEQ ID NO: 2 comprises the amino acid sequence of the Yap gene product.

Methods and compositions described herein require that the levels and/or activity of WW domain containing transcription regulator 1 (WWTR1) is inhibited. As used herein, WWTR1, also known as Taz, refers to a transcriptional coactivator with PDZ-binding motif. WWTR1 sequences are known for a number of species, e.g., human WWTR1 (NCBI Gene ID: 25937) polypeptide (e.g., NCBI Ref Seq NP_001161750.1) and mRNA (e.g., NCBI Ref Seq NM_001168278.2). WWTR1 can refer to human WWTR1, including naturally occurring variants, molecules, and alleles thereof. WWTR1 refers to the mammalian WWTR1 of, e.g., mouse, rat, rabbit, dog, cat, cow, horse, pig, and the like. The nucleic sequence of SEQ ID NO: 3 comprises a nucleic sequence which encodes WWTR1. The amino acid sequence of SEQ ID NO: 4 comprises the amino acid sequence of the WWTR1 gene product.

As used herein, a “reference level” refers to a normal, otherwise unaffected cell population, e.g., a T cell population, or tissue (e.g., a biological sample obtained from a healthy subject, or a biological sample obtained from the subject at a prior time point, e.g., a biological sample obtained from a patient prior to being diagnosed with a disease or disorder, or a biological sample that has not been contacted with an agent disclosed herein).

As used herein, an “appropriate control” refers to an untreated, otherwise identical cell (e.g., a T cell) population (e.g., obtained patient who was not administered an agent or compositions described herein, or was administered by only a subset of agents described herein, as compared to a non-control cell).

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean±1%.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Other terms are defined within the description of the various aspects and embodiments of the technology of the following.

### BRIEF DESCRIPTION OF THE DRAWINGS

This application file contains at least one drawing executed in color. Copies of this patent application publication with color drawings will be provided by the Office upon request and payment of the necessary fee.

FIGS. 1A-1J show that Yap expression is induced upon T-cell activation resulting in suppression of T-cell activation. WT and Yap-cKO CD4+ and CD8+ T cells were isolated from mouse spleens and stimulated with anti-CD3 and anti-CD28 coated magnetic beads to test expression of Yap protein and TEAD1-4 mRNA. T-cell activation was tested using increasing concentrations of plate-bound anti-CD3 with soluble anti-CD28 in Yap-cKO and WT T cells. Activation marker expression was also tested in WT CD4+ and CD8+ T cells treated with increasing concentrations of verteporfin under IL-2, and CD3/CD28 stimulation. Statistical differences were determined by using an F test to identify differences between nonlinear curve fits (FIGS. 1C-1F), unpaired two-sample t test (FIGS. 1G-1H), or one-way repeated-measures ANOVA with post hoc test for linear trend with increasing verteporfin (FIGS. 1I-1J). The underlying data for the graphs in this figure can be found in data presented herein and for the immunoblots in Raw Images. (FIG. 1A) Yap protein levels in CD4+ T cells isolated from WT mouse spleens at various time points following CD3/CD28 stimulation. (FIG. 1B) Yap protein levels in CD8+ T cells isolated from WT mouse spleens at various time points following CD3/CD28 stimulation. (FIG. 1C) CD44 expression by flow cytometry on WT and Yap-cKO CD4+ T cells 72 hours post CD3/CD28 stimulation (n=2-3 per dose/group). (FIG. 1D) CD44 expression by flow cytometry on WT and Yap-cKO CD8+ T cells 72 hours post CD3/CD28 stimulation (n=2-3 per dose/group). (FIG. 1E) CD25 expression by flow cytometry on WT and Yap-cKO CD4+ T cells 72 hours post CD3/CD28 stimulation (n=2-3 per dose/group). (FIG. 1F) CD25 expression by flow cytometry on WT and Yap-cKO CD8+ T cells 72 hours post CD3/CD28 stimulation (n=2-3 per dose/group). (FIG. 1G) TEAD1-4 mRNA expression in CD4+ T cells 24 hours post CD3/CD28 stimulation (n=3/group). (FIG. 1H) TEAD1-4 mRNA expression in CD8+ T cells 24 hours post CD3/CD28 stimulation (n=3/group). (FIG. 1I) CD71 expression on WT CD4+ T cells 72 hours post IL-2 and CD3/CD28 stimulation (Stim) and increasing concentration of verteporfin (n=4/group). (FIG. 1J) CD71 expression on WT CD8+ T cells 72 hours post IL-2 and CD3/CD28 stimulation (Stim) and increasing concentration of verteporfin (n=4/group). CD3, cluster of differentiation 3; CD, cluster of differentiation, cKO, conditional knockout; GAPDH, glyceraldehyde 3-phosphate dehydrogenase; IL-2, interleukin 2; MFI, median fluorescence intensity; No Stim, no stimulation; Stim, stimulation with IL-2 and anti-CD3/CD28; TEAD, TEA domain family member; WT, wild type; Yap, Yes-associated protein

FIGS. 2A-2D show that deletion of Yap in CD4+ T cells results in increased IFNγ, IL-17, GATA3, and Foxp3 expression under Th1-, Th17-, Th2-, and Treg-polarizing conditions, respectively. Naïve CD4+ T cells from WT and Yap-cKO mice were isolated using magnetic beads. WT and Yap-cKO T cells were cultured under Th1-, Th17-, Th2-, or Treg-polarizing conditions for 5 days in the presence of CD3 and CD28 antibodies. On day 5, IFNγ, IL-17, GATA3, and Foxp3 expression were measured using flow cytometry. Statistical differences were determined by using a Student t test (FIGS. 2A-2C) or an F test to identify differences between nonlinear curve fits (FIG. 2D). The underlying data for the graphs in this figure can be found in S2 Data. (FIG. 2A) IFNγ and IL-17 expression in WT and Yap-cKO CD4⁺ T cells under Th1-polarizing conditions (n=3/group). (FIG. 2B) IFNγ and IL-17 expression in WT and Yap-cKO CD4+ T cells under Th17-polarizing conditions (n=3/group). (FIG. 2C) IFNγ and GATA3 expression in WT and Yap-cKO CD4+ T cells under Th2-polarizing conditions (n=3/group). (FIG. 2D) CD25 and Foxp3 expression in WT and Yap-cKO CD4⁺ T cells under Treg-polarizing conditions (n=3-6 per dose/group). CD, cluster of differentiation; cKO, conditional knockout; Foxp3, forkhead box protein 3; GATA3, GATA binding protein 3; IFNγ, interferon gamma; Iono, ionomycin; IL-17, interleukin 17; No Stim, no stimulation; PMA, phorbol 12-myristate 13-acetate; TGFβ, transforming growth factor beta; Th, T helper cell type; Treg, regulatory T cell; WT, wild type; Yap, Yes-associated protein.

FIGS. 3A-3F show that Thymocyte development is similar between WT and Yap-cKO T cells. (FIG. 3A) Total thymocytes were isolated from thymuses of WT and Yap-cKO mice. Cells were stained and analyzed by flow cytometry for coreceptor maturation (FIG. 3A), positive selection (FIG. 3B), medullary maturation (FIGS. 3C-3E), and negative selection (FIG. 3F). Statistical differences were determined using two-way ANOVA followed by Sidak's pairwise multiple comparisons tests. The underlying data for the graphs in this figure can be found in S3 Data. (FIG. 3A) Absolute numbers of thymocytes in different maturation stages: DN, DP, and SP for CD4 and CD8 coreceptor expression (n=6-7 mice per group). (FIG. 3B) Frequency of thymocytes progressing through positive selection determined by TCRβ and CD69 expression (n=6-7 mice per group). (FIG. 3C) Frequency of TCRβ+CCR7+SP thymocytes in SM stage (CD69+MHCI−) (n=6-7 mice per group). (FIG. 3D) Frequency of TCRβ+CCR7+SP thymocytes in M1 stage (CD69+MHCI+) (n=6-7 mice per group). (FIG. 3E) Frequency of TCRβ+CCR7+SP thymocytes in M2 stage (CD69-MHCI+) (n=6-7 mice per group). (FIG. 3F) Frequency of Nur77+ cells among DP, CD4SP, or CD8SP thymocytes (n=6-7 mice per group). CCR, chemokine receptor; CD, cluster of differentiation; cKO, conditional knockout; DN, double negative; DP, double positive; MHCI, major histocompatibility complex class I; M1, mature 1; M2, mature 2; N.S., not significant; Nur77, nuclear receptor 77; pre-sel., pre-selection; pos. sel., positive selection; SM, semimature; SP, single positive; TCR, T cell receptor; WT, wild type; Yap, Yes-associated protein

FIGS. 4A-4K show that T-cell-specific deletion of Yap results in reduced tumor growth and enhanced T-cell tumor infiltration. Mice were challenged subcutaneously with B16F10 or LLC tumor cells on the right flank. Some mice carrying B16F10 tumors received adoptive cell transfer of WT and Yap-cKO CD8+ T cells. Tumor growth was monitored over the course of 15 days, until the maximum size of the tumors reached 500 mm3. B16F10 tumors were harvested for immunofluorescence or flow cytometric analysis. Statistical differences were determined by using a Student t test. The underlying data for the graphs in this figure can be found in data presented herein. (FIG. 4A) B16 tumor growth curve of WT and Yap-cKO mice (n=9/group). (FIG. 4B) Tumor weight of B16 tumors derived from WT and Yap-cKO mice on day 15 post injection (n=9/group). (FIG. 4C) LLC tumor growth curve of WT and Yap-cKO mice (n=7/WT group, n=5/cKO group). (FIG. 4D) Tumor weight of LLC tumors derived from WT and Yap-cKO mice on day 15 post injection (n=7/WT group, n=5/cKO group). (FIG. 4E) CD8+ T-cell immunofluorescence on day 15 of B16 tumor growth. (FIG. 4F) Absolute numbers of CD3+ TILs from WT and Yap-cKO B16 tumors. Tumors were harvested on day 15; stained with antibodies against CD45, CD3, CD4, and CD8; and analyzed using flow cytometry (n=5/WT mice, n=4/Yap-cKO mice). (FIG. 4G) Absolute numbers of CD4+ TILs from WT and Yap-cKO B16 tumors, prepared as in FIG. 4D (n=5/WT mice, n=4/Yap-cKO mice). (FIG. 4H) Absolute numbers of CD8+ TILs from WT and Yap-cKO B16 tumors, prepared as in FIG. 4D (n=5/WT mice, n=4/Yap-cKO mice). (FIG. 4I) Experimental plan for adoptive cell transfer of WT and Yap-cKO CD8+ T cells in WT B16-bearing mice. (FIG. 4J) Absolute numbers of dTom+ and EYFP+ Yap-cKO versus WT CD8+ T cells in C57BL/6 B16 tumors. WT dTom+CD8+ T cells were mixed 1:1 with Yap-cKO EYFP+CD8+ T cells prior to being injected into WT C57BL/6 mice. Subsequently, mice were injected subcutaneously with B16F10 melanoma cells, and absolute number of infiltrating T cells was determined on day 15 by flow cytometry (n=5/group). (FIG. 4K) Percentage of dTom+ and EYFP+CD8+ T cells out of total B16 tumor-infiltrating CD8+ T cells (n=5/group). CD, cluster of differentiation; cKO, conditional knockout; CTL, Control; dTom, dTomato; EYFP, enhanced yellow fluorescent protein; LLC, Lewis lung carcinoma; TIL, tumor-infiltrating lymphocyte; WT, wild type; Yap, Yes-associated protein

FIGS. 5A-5L show a RNA-seq analysis of Yap-cKO CD4+ and CD8+B16 TILs uncovers distinct gene expression changes that correlate with T-cell tumor infiltration. CD4+ and CD8+ TILs and TDLNs were isolated from WT and Yap-cKO mice challenged with B16F10 tumors, and gene expression was analyzed by RNA-seq in the respective cells. RNA-seq data can be found at the NCBI GEO (Series Accession Number GSE139883) and data not shown. The hyper-enrichment results are outlined in Table 3. (FIG. 5A) Heatmap showing DEGs identified from Yap-cKO versus WT CD4+ TILs. (FIG. 5B) Yap-cKO versus WT CD8+ TIL DEG heatmap. (FIG. 5C) Yap-cKO versus WT CD4+ TDLN DEG heatmap. (FIG. 5D) Yap-cKO versus WT CD8+ TDLN DEG heatmap. (FIG. 5E) Hyper-enrichment analysis shows enrichment of induced gene sets observed in CD4+ Yap-cKO TILs. (FIG. 5F) Hyper-enrichment analysis shows enrichment of induced gene sets observed in CD8+ Yap-cKO TILs. (FIG. 5G) Hyper-enrichment analysis shows enrichment of repressed gene sets observed in CD4+ Yap-cKO TILs. (FIG. 5H) Hyper-enrichment analysis shows enrichment of repressed gene sets observed in CD8+ Yap-cKO TILs. (FIG. 5I) Gene expression changes identified in Yap-cKO CD4+ and CD8+ TILs correlate with genes reflecting tumor infiltration across many cancers in TCGA data. The heatmap is colored by the coefficient, and the text of each cell represents the adjusted p-value of the correlation. (FIG. 5J) Gene expression changes identified in Yap-cKO CD4+ and CD8+ T cells correlate with patient survival data available in TCGA across several cancers. Red: the average survival probability is higher for patients with high activity of the signature. Blue: the average survival probability is higher for patients with low activity of the signature. Each cell includes the p-value for the survival estimation. The distribution of p-values arising from the multiple survival analyses for each signature across TCGA datasets was compared to a uniform distribution using a Kolmogorov-Smirnov test. (FIG. 5K) Kaplan-Meier survival analysis showing the average survival probability of patients with LUAD that show low versus high Yap activity derived from the Yap-cKO CD4+ gene expression signature. (FIG. 5L) Kaplan-Meier survival analysis showing the average survival probability of patients with LUAD that show low versus high Yap activity derived from the Yap-cKO CD8+ gene expression signature. ACC, adrenocortical carcinoma; BLCA, bladder urothelial carcinoma; BRCA, breast invasive carcinoma; CD, cluster of differentiation; CESC, cervical squamous cell carcinoma and endocervical adenocarcinoma; CHOL, cholangiocarcinoma; cKO, conditional knockout; COAD, colon adenocarcinoma; DEG, differentially expressed gene; lymphoid neoplasm diffuse large B-cell lymphoma; DLN, draining lymph node; ESCA, esophageal carcinoma; GBM, glioblastoma multiforme; GEO, Gene Expression Omnibus; HNSC, head and neck squamous cell carcinoma; KICH, kidney chromophobe; KIRC, kidney renal clear cell carcinoma; KIRP, kidney renal papillary cell carcinoma; k.s., Kolmogorov-Smirnov; LGG, brain lower grade glioma; LIHC, liver hepatocellular carcinoma; LUAD, lung adenocarcinoma; LUSC, lung squamous cell carcinoma; MESO, mesothelioma; NCBI, National Center for Biotechnology Information; OV, ovarian serous cystadenocarcinoma; PAAD, pancreatic adenocarcinoma; PCPG, pheochromocytoma and paraganglioma; PRAD, prostate adenocarcinoma; READ, rectum adenocarcinoma; RNA-seq, RNA sequencing; SARC, sarcoma; SKCM, skin cutaneous melanoma; STAD, stomach adenocarcinoma; TCGA, The Cancer Genome Atlas; TDLN, tumor-draining lymph node; TGCT, testicular germ cell tumors; THCA, thyroid carcinoma; THYM, thymoma; TIL, tumor-infiltrating lymphocyte; UCEC, uterine corpus endometrial carcinoma; UCS, uterine carcinosarcoma; UVM, uveal melanoma; WT, wild type; Yap, Yes-associated protein

FIGS. 6A-6K show that Yap deletion or pharmacological inhibition of Yap does not significantly affect T-cell proliferation. CD4+ and CD8+ T cells were isolated from WT or Yap-cKO mice and screened for EYFP expression as well as activation marker expression and proliferation after αCD3 and αCD28 stimulation. Proliferation was also tested for WT CD4+ and CD8+ T cells treated with increasing concentrations of verteporfin under IL-2, anti-CD3, and anti-CD28 stimulation. Statistical differences were determined by using a Student t test, with “ns” indicating not significant. (FIG. 6A) Yap mRNA expression in WT or Yap-cKO CD4+ T cells following CD3/CD28 stimulation. (FIG. 6B) Yap mRNA expression in WT or Yap-cKO CD8+ T cells following CD3/CD28 stimulation. (FIG. 6C) EYFP expression by flow cytometry on CD4+ cells isolated from WT or Yap-cKO mouse spleens. (FIG. 6D) EYFP expression by flow cytometry on CD8+ cells isolated from WT or Yap-cKO mouse spleens. (FIG. 6E) CD69 expression on WT and Yap-cKO CD4+ T cells 72 hours post CD3/CD28 stimulation (n=2-3 per dose/group). (FIG. 6F) CD69 expression on WT and Yap-cKO CD8+ T cells 72 hours post CD3/CD28 stimulation (n=2-3 per dose/group). (FIG. 6G) WT and Yap-cKO CD4+ T-cell proliferation (n=3/group). (FIG. 6H) WT and Yap-cKO CD8+ T-cell proliferation (n=3/group). (FIG. 6I) CD69 expression on WT CD4+ T cells 72 hours post IL-2 and CD3/CD28 stimulation and increasing concentration of verteporfin (n=4/group). (FIG. 6J) CD69 expression on WT CD4+ T cells 72 hours post IL-2 and CD3/CD28 stimulation and increasing concentration of verteporfin (n=4/group). (FIG. 6K) Proliferation of DMSO-versus verteporfin-treated WT CD4+ and CD8+ T cells (representative of 4 independent experiments). Raw data for this experiment are available in FLOWRepository (Repository ID: FR-FCM-Z2D5).

FIGS. 7A-7B show the top 25 up- and down-regulated genes responding to Yap deletion in CD4+ and CD8+ TILs. RNA-seq was performed from CD4+ and CD8+ TILs and TDLNs that were isolated from WT and Yap-cKO mice challenged with B16F10 tumors (data at NCBI GEO GSE139883 and data not shown herein), and the top DEGs are shown. (FIG. 7A) A heatmap representing the top and bottom 25 DEGs in Yap-cKO versus WT CD4+ TILs. (FIG. 7B) A heatmap representing the top and bottom 25 DEGs in Yap-cKO versus WT CD8+ TILs.

FIGS. 8A-8H show the expression of genes related to T-cell activation, chemokines and chemokine receptors, and T-helper subset-defining factors are up-regulated in Yap-cKO CD4+ and CD8+TILs. DEGs identified in Yap-cKO versus WT CD4+ and CD8+ TILs that encode factors related to T-cell function are shown. These data were derived from RNA-seq analysis of the respective mice challenged with B16F10 tumors, which is available at NCBI GEO (GSE139883) and in data not shown. (FIG. 8A) Log 10(normalized RNA-seq counts+1) of T-cell activation-related genes in Yap-cKO versus WT CD8+ TILs. (FIG. 8B) Log 10(normalized RNA-seq counts+1) of T-cell activation-related genes in Yap-cKO versus WT CD4+ TILs. (FIG. 8C) Log 10(normalized RNA-seq counts+1) of chemokine genes in Yap-cKO versus WT CD8+ TILs. (FIG. 8D) Log 10(normalized RNA-seq counts+1) of chemokine receptor genes in Yap-cKO versus WT CD8+ TILs. (FIG. 8E) Log 10(normalized RNA-seq counts+1) of chemokine genes in Yap-cKO versus WT CD4+ TILs. (FIG. 8F) Log 10(normalized RNA-seq counts+1) of chemokine receptor genes in Yap-cKO versus WT CD4+ TILs. (FIG. 8G) Log 10(normalized RNA-seq counts+1) of T-helper subset-defining cytokines in Yap-cKO versus WT CD4+ TILs. (FIG. 8H) Log 10(normalized RNA-seq counts+1) of T-helper subset-defining transcription factors in Yap-cKO versus WT CD4+ TILs. Significant differences were determined by a Student t test; *p<0.05; **p<0.01; ***p<0.001.

FIGS. 9A-9D show that Yap-cKO TILs are skewed towards Th2 and Treg gene expression signatures compared to WT. DEGs identified in Yap-cKO versus WT CD4+ TILs that represent different CD4+ fates are shown. These data were derived from RNA-seq analysis of the respective mice challenged with B16F10 tumors, which is available at NCBI GEO (GSE139883) and data not shown. (FIG. 9A) Heatmap of statistically significant differentially expressed Th1-related genes in Yap-cKO versus WT CD4+ TILs. (FIG. 9B) Heatmap of statistically significant differentially expressed Th2-related genes in Yap-cKO versus WT CD4+ TILs. (FIG. 9C) Heatmap of statistically significant differentially expressed Th17-related genes in Yap-cKO versus WT CD4+ TILs. (FIG. 9D) Heatmap of statistically significant differentially expressed Treg-related genes in Yap-cKO versus WT CD4+ TILs.

FIGS. 10A-10B show that the TEAD-binding motif is enriched in upstream regulatory elements found in genes altered in expression within Yap-deleted TILs. HOMER de novo motif analysis was performed on down-regulated gene expression changes identified in Yap-cKO versus WT (FIG. 10A) CD4+ and (FIG. 10B) CD8+ TILs, revealing the TEAD transcription factor motifs among the top enriched motifs.

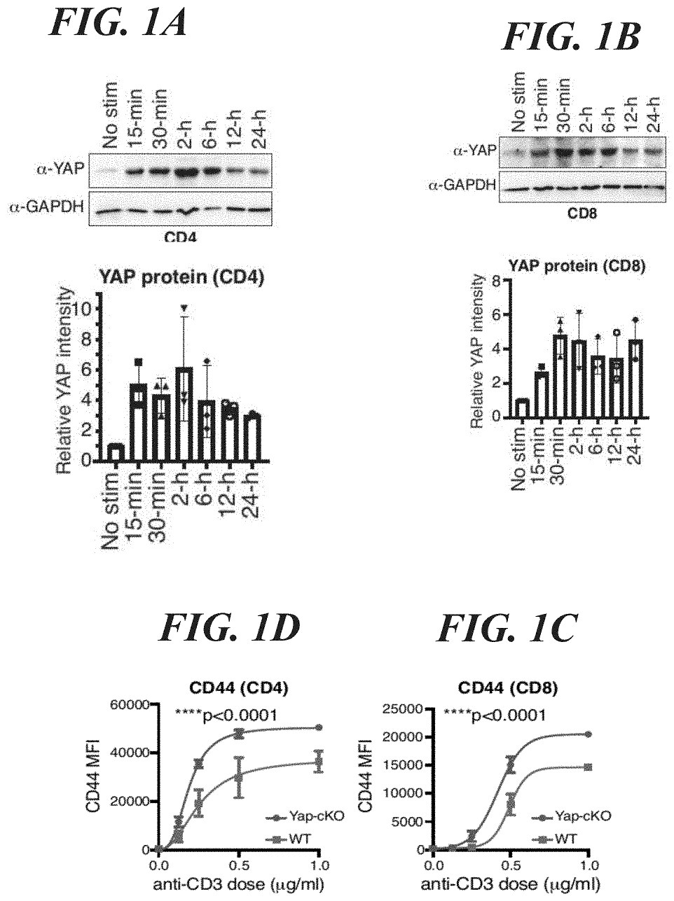

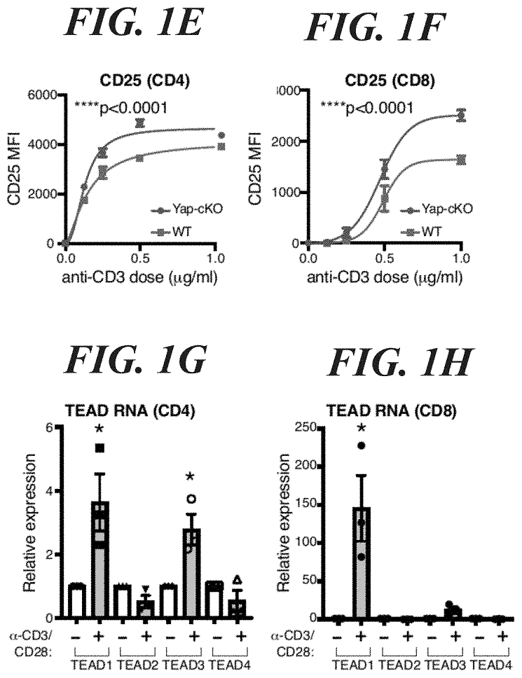

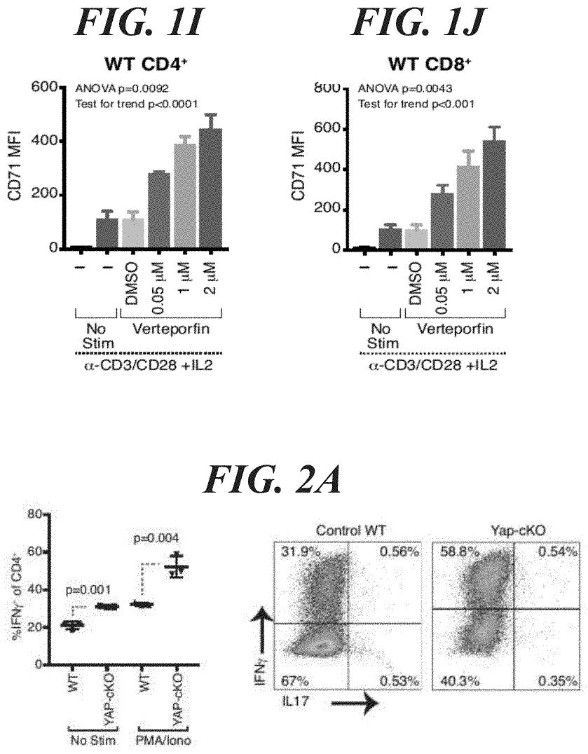

FIGS. 1A-11F show T cell specific YAP deletion results in enhanced T cell activation but not proliferation. WT and YAP-cKO CD4+ and CD8+ T cells were isolated from mouse spleens and stimulated with αCD3 and αCD28 coated magnetic beads to test expression of activation markers and proliferation. Activation marker expression and proliferation was also tested for WT CD4+ and CD8⁺ T cells treated with increasing concentrations of Verteporfin under IL2 and αCD3 and αCD28 stimulation. (For a-f, significant differences were determined by the Student t test; *, p<0.05; **, p<0.01; ***, p<0.001; ****, p<0.0001; ns, not significant). FIG. 11A shows WT and YAP-cKO CD4+ T cells were isolated from mouse spleens and stimulated with αCD3 and αCD28 coated magnetic beads. CD4+ T cell expression of CD25 was measured 24 hours later (n=3/group). FIG. 11B shows WT and YAP-cKO CD8+ T cells were isolated from mouse spleens and stimulated with αCD3 and αCD28 coated magnetic beads. CD8+ T cell expression of CD25 was measured 24 hours later (n=4/group). FIG. 11C shows cells were treated as in FIG. 11A. CD4+ T cell expression of CD69 was measured 24 hours later (n=3/group). FIG. 11D shows cells were treated as in FIG. 11B. CD8+ T cell expression of CD69 was measured 24 hours later (n=4/group). FIG. 11E shows WT CD4+ T cells were isolated from mouse spleens and stimulated with αCD3 and αCD28 coated magnetic beads in the presence of IL2 and increasing concentration of verteporfin. CD4+ T cell expression of CD69 was measured 3 days later. FIG. 11F shows WT CD8+ T cells were isolated from mouse spleens and stimulated with αCD3 and αCD28 coated magnetic beads in the presence of IL2 and increasing concentration of verteporfin. CD8+ T cell expression of CD69 was measured 3 days later.

FIGS. 12A-12B show a RNA-Seq analysis of WT and CD4Cre YAP KO CD4+ and CD8+B16 TILs uncovers YAP as a negative regulator of T cell activation, differentiation, cytokine signaling and migration. Kaplan-Meier Survival analysis showing the average survival probability of patients with lung adenocarcinoma that show low versus high YAP activity derived from the YAP-cKO CD4+ gene expression signature. FIG. 12A shows gene expression changes identified in YAP-cKO CD4+ and CD8+ TILs correlate with genes reflecting tumor infiltration across many cancers in TCGA data. CD4+ and CD8+ TIL signature activation was defined as the sum of activation of the upregulated signature and inactivation of the downregulated signature and was calculated using Gene Set Variation Analysis (GSVA) in primary tumor samples across TCGA. The Tumor IMmune Estimation Resource (TIMER (17)) was used to estimate the abundance of CD4+ T Cell and CD8+ T cells in the tumor microenvironment in a series of TCGA datasets. The heatmap is colored by the correlation value and the text of each cell represents the adjusted p-value. FIG. 12B shows gene expression changes identified in YAP-cKO CD4+ and CD8+ T cells correlate with patient survival data available in TCGA across several cancers. CD4+ and CD8+ TIL signature activation was defined as the sum of activation of the upregulated signature and inactivation of the downregulated signature and was used to stratify patients across TCGA datasets into high or low activity. For each data set, Kaplan-Meir survival analysis was performed and the results were summarized with a heatmap. Blue: the average survival probability for patients with low activity of the signature is higher. Red: the average survival probability for patients with high activity of the signature is higher. Yellow: neither group has a higher probability of survival. The text of each cell is the p-value for the survival estimation. The distribution of p-values arising from the multiple survival analyses for each signature across TCGA datasets was compared to a uniform distribution using a Kolmogorov-Smimov test.

FIGS. 13A-13K show that the deletion of YAP in T cells leads to enhanced generation of lung CD8+ resident memory T cells in response to influenza infection. WT and YAP-cKO mice were infected with 500 FFU influenza (strain PR8) or were treated with saline. Eight weeks later, lungs were harvested for CD8+ T cell phenotyping. (For a-k, significant differences were determined by the Student t test; *, p<0.05; **, p<0.01; ***, p<0.001; ****, p<0.0001; ns, not significant). (n=4/WT saline, n=3/cKO saline, n=3/WT PR8, n=4/cKO PR8). FIG. 13A shows percentage of CD8+ T cells of CD45+ lung infiltrating cells from the lungs of uninfected and influenza infected WT and YAP-cKO mice. FIG. 13B shows percentage of CD69+CD8+ lung infiltrating T cells in uninfected and influenza infected WT and YAP-cKO mice. FIG. 13C shows percentage of CD62L+CD8+ lung infiltrating T cells in uninfected and influenza infected WT and YAP-cKO mice. FIG. 13D shows percentage of CD44+CD8+ lung infiltrating T cells in uninfected and influenza infected WT and YAP-cKO mice. FIG. 13E shows percentage of CD44-CD62L+(naïve) CD8+ lung infiltrating T cells in uninfected and influenza infected WT and YAP-cKO mice. FIG. 13F shows percentage of CD44+CD62L− (effector memory) CD8+ lung infiltrating T cells in uninfected and influenza infected WT and YAP-cKO mice. FIG. 13G shows percentage of CD44+CD62L−CD103+CD69+(resident memory) CD8+ lung infiltrating T cells in uninfected and influenza infected WT and YAP-cKO mice. FIG. 13H shows absolute number of CD8+CD69+ lung infiltrating T cells in uninfected and influenza infected WT and YAP-cKO mice. FIG. 13I shows absolute number of CD8+CD44+ lung infiltrating T cells in uninfected and influenza infected WT and YAP-cKO mice. FIG. 13J shows absolute number of CD8+CD44+CD62L+ lung infiltrating T cells in uninfected and influenza infected WT and YAP-cKO mice. FIG. 13K shows absolute number of CD8+CD44+CD62L-CD103+CD69+ lung infiltrating T cells in uninfected and influenza infected WT and YAP-cKO mice.

FIGS. 14A-14K show that CD4Cre YAP KO does not lead to enhanced generation of lung CD4+ resident memory T cells in response to influenza infection. WT and YAP-cKO mice were infected with 500 FFU influenza (strain PR8) or were treated with saline. Eight weeks later, lungs were harvested for CD4+ T cell phenotyping. (For FIGS. 14A-14K, significant differences were determined by the Student t test; *, p<0.05; **, p<0.01; ***, p<0.001; ****, p<0.0001; ns, not significant). (n=4/WT saline, n=3/cKO saline, n=3/WT PR8, n=4/cKO PR8). FIG. 14A shows percentage of CD4+ T cells of CD45+ lung infiltrating cells from the lungs of uninfected and influenza infected WT and YAP-cKO mice. FIG. 14B shows percentage of CD69+CD4+ lung infiltrating T cells in uninfected and influenza infected WT and YAP-cKO mice. FIG. 14C shows percentage of CD62L+CD4+ lung infiltrating T cells in uninfected and influenza infected WT and YAP-cKO mice. FIG. 14D shows percentage of CD44+CD4+ lung infiltrating T cells in uninfected and influenza infected WT and YAP-cKO mice. FIG. 14D shows the shows the percentage of CD44+CD4+ lung infiltrating T cells in uninfected and influenza infected WT and YAP-cKO mice. FIG. 14E shows the percentage of CD44+-CD621+(naïve) CD4+ lung infiltrating T cells in uninfected and influenza infected WT and YAP-cKO mice. FIG. 14F shows the percentage of CD44±CD62L− (effector memory) CD4+ lung infiltrating T cells in uninfected and influenza infected WT and YAP-cKO mice. FIG. 14.G. Percentage of CD44+CD62L-CD11A+CD69+(resident memory) CD4+ lung infiltrating T cells in uninfected and influenza infected WT and YAP-cKO mice. FIG. 14H. Absolute number of CD4+CD69+ lung infiltrating T cells in uninfected and influenza infected WT and YAP-cKO mice. FIG. 14I. Absolute number of CD4+CD44+ lung infiltrating T cells in uninfected and influenza infected WT and YAP-cKO mice. FIG. 14J. Absolute number of CD4+CD44+CD62L+ lung infiltrating T cells in uninfected and influenza infected WT and YAP-cKO mice. FIG. 14K. Absolute number of CD4+CD44+CD62L−CD11A+CD69+ lung infiltrating T cells in uninfected and influenza infected WT and YAP-cKO mice.

FIGS. 15A-15F shows deletion of YAP in T cells does not impact the formation of lung CD8+ resident memory T cells in response to pneumonia infection. WT and YAP-cKO mice were infected with S. pneumoniae, serotype 19 (SP19). Eight weeks later, lungs were harvested for CD8+ T cell phenotyping. (For a-f, significant differences were determined by the Student t test; *, p<0.05; **, p<0.01; ***, p<0.001; ****, p<0.0001; ns, not significant). (n=3/WT SP19, n=4/cKO SP19). FIG. 15A shows percentage of CD8+ T cells of CD45+ lung infiltrating cells from the lungs of SP19 infected WT and YAP-cKO mice. FIG. 15B shows percentage of CD69+CD8+ lung infiltrating T cells in uninfected and influenza infected WT and YAP-cKO mice. FIG. 15C shows percentage of CD62L+CD8+ lung infiltrating T cells in uninfected and influenza infected WT and YAP-cKO mice. FIG. 15D shows percentage of CD44+CD8+ lung infiltrating T cells in uninfected and influenza infected WT and YAP-cKO mice. FIG. 15E shows percentage of CD44+CD62L− (effector memory) CD8+ lung infiltrating T cells in uninfected and influenza infected WT and YAP-cKO mice. FIG. 15F shows percentage of CD44+CD62L-CD103+CD69+(resident memory) CD8+ lung infiltrating T cells in uninfected and influenza infected WT and YAP-cKO mice.

FIGS. 16A-16F show that the deletion of YAP in T cells leads to enhanced generation of lung CD4+ resident memory T cells in response to pneumonia infection. WT and YAP-cKO mice were infected with S. pneumoniae, serotype 19 (SP19). Eight weeks later, lungs were harvested for CD4+ T cell phenotyping. (For a-f, significant differences were determined by the Student t test; *, p<0.05; **, p<0.01; ***, p<0.001; ****, p<0.0001; ns, not significant). (n=3/WT SP19, n=4/cKO SP19). FIG. 16A shows percentage of CD4+ T cells of CD45+ lung infiltrating cells from the lungs of uninfected and influenza infected WT and YAP-cKO mice. FIG. 16B shows percentage of CD69+CD4+ lung infiltrating T cells in uninfected and influenza infected WT and YAP-cKO mice. FIG. 16C shows percentage of CD62L+CD4+ lung infiltrating T cells in uninfected and influenza infected WT and YAP-cKO mice. FIG. 16D shows percentage of CD44+CD4+ lung infiltrating T cells in uninfected and influenza infected WT and YAP-cKO mice. FIG. 16E shows percentage of CD44+CD62L− (effector memory) CD4+ lung infiltrating T cells in uninfected and influenza infected WT and YAP-cKO mice. FIG. 16F shows percentage of CD44+CD62L−CD11A+CD69+(resident memory) CD4+ lung infiltrating T cells in uninfected and influenza infected WT and YAP-cKO mice.

FIGS. 17A-17D show that T helper subset defining cytokines and transcription factors are upregulated in YAP-cKO CD4+ TILs, with TH2 and Treg signatures being enriched in YAP-cKO vs WT CD4+ TILs. FIG. 17A shows heatmap of statistically significant differentially expressed TH1 genes in WT vs YAP-cKO CD4+ TILs. FIG. 17B shows heatmap of statistically significant differentially expressed TH2 genes in WT vs YAP-cKO CD4+ TILs. FIG. 17C shows heatmap of statistically significant differentially expressed TH17 genes in WT vs YAP-cKO CD4+ TILs. FIG. 17D shows heatmap of statistically significant differentially expressed Treg genes in WT vs YAP-cKO CD4+ TILs.

FIGS. 18A-18B show that shows Yap-deleted CD8+ T cells exhibit enhanced B16 tumor cell killing activity in vitro. Yap-loxP/loxP; CD4-Cre; Thy1a; Tcra-V1/Tcrb-V13 (Yap-cko-pmel) C57BL/6 mice were generated, resulting in deletion of the Yap gene in CD4+ and CD8+ T cells of a mouse that carries a transgenic TCR that recognizes an epitope found on the poorly immunogenic B16 melanoma cell line. CD8+ T-cells were purified from splenocytes of control pmel mice (CD4-Cre; Thy1a; Tcra-V1/Tcrb-V13) and Yap-cko-pmel mice and then activated using anti-CD3/anti-CD28 coated Dynabeads at a 1:1 ratio for 48 hours. These CD8+ T cells were then mixed and co-cultured with B16 melanoma cells expressing Nano-Luciferase (B16-NanoLuc) growing in either 2D or 3D culture conditions. For 2D culture condition, B16-NanoLuc cells were seeded in 96-well plate overnight at a density of 5×10³ cells per well. On day 0, T cells were cocultured with B16-NanoLuc cells at 1:1 ratio. B16 cell number was quantified 48 hours after co-culturing with T cells. For 3D culture condition, 96-well plate was coated with 50 ul 1.5% agar per well. 5×10³ B16 cells were cultured for 72 hours, which allowed the cells to form spheroids. These spheroids were then co-cultured with T cells at 1:1 ratio and B16-NanoLuc cell number was quantified after 24 hours of co-culture. The growth of B16 was quantified using Nano-Glo® Luciferase Assay System (Promega). Results shown in FIG. 18A were normalized by cell counts at day 0. FIG. 18A shows B16-NanoLuc cells growing in 2D culture were co-cultured with control (WT) and Yap-cko-pmel T cells and the relative number of B16-NanoLuc cells were measured following 48 hours of co-culture. FIG. 18B shows B16-NanoLuc cells were seeded in 96-well plate coated with agar for 3 days to form spheroids. Pmel CD8+ T-cells were pre-activated for 5 days, and cocultured with B16 cells for 24 hours. Results were normalized by day 0 luciferase reading. Each datapoint represents a separate experimental well (n=6). A student's t-test was used to determine statistical significance.

FIGS. 19A-19C show that Yap-deleted T cells exhibit higher activation, expansion, markers of tissue homing memory, and Th1-skewed cytokines when grown on different stiffness hydrogels in vitro. Mouse CD8+ T cells were purified from splenocytes of C57BL/6J (WT) or CD4-Cre YAP-floxed C57BL/6J (YAP-cKO) mice. Cells were cultured either on hydrogels or uncoated 96-well tissue culture plates. Hydrogels were tested at low (0.5 kPa) and medium (1 kPa) stiffnesses. Hyaluronic acid-based hydrogels incorporated covalently conjugated anti-CD3, anti-CD28, and IL-2-Ig on the surface. Cells cultured on uncoated culture plates were incubated 1:1 with anti-CD3/anti-CD28-coated Dynabeads. Cells were activated for 7 days and analyzed for cell expansion compared to seeding density at day 0 (FIG. 19A). Cell activation/tissue homing memory was determined by sustained CD69 levels, stained and analyzed by flow cytometry (FIG. 19B). The Th1/Th2 cytokine milieu was quantified using Luminex, adjusted for cell density, and normalized as log 2 fold-change YAP-cKO compared to WT controls (FIG. 19C).

FIG. 20 shows that the YAP paralog TAZ (WWTR1) may be a synergistic immunomotherapy target with YAP following acute YAP-inhibition. Pan (CD4+ and CD8+) T cells were isolated from splenocytes of healthy C57BL/6J mice. Scrambled siRNA or YAP1 siRNA (Horizon Discovery) were delivered to freshly isolated T cells by nucleofection (Amaxa). Nucleofected T cells were plated and activated overnight with 1:1 anti-CD3/anti-CD28−-coated Dynabeads. Cells were collected at 24 h post-activation and RNA purified. Yap1 and Wwtr1 expression were determined using real-time RT-PCR Tagman probes and normalized to internal Actb control probes. Knockdown of YAP and upregulation of TAZ in activated T cells with YAP siRNA was calculated by normalization to activated T cells with scrambled siRNA (FIG. 20). Shown are results from 3 independent experiments.

### DETAILED DESCRIPTION

### Methods for Activating or Differentiating T-Cells

Data presented herein show that Yap levels are elevated upon T-cell activation and conditional deletion of the Yap gene in CD4+ and CD8+ T cells enhances their activation and differentiation potential. These phenotypes translated in vivo to reduced growth of B16F10 melanoma and Lewis lung carcinoma (LLC) lung cancer tumors in mice with Yap-deleted T cells, with these mice showing notably increased T-cell infiltration into tumors. Yap-deleted polyclonal CD8+ T cells have an intrinsic ability to infiltrate tumors with higher efficiency. RNA sequencing (RNA-seq) analyses of Yap-deleted tumor-infiltrating lymphocytes (TILs) revealed up-regulation of key signals important for CD4+ and CD8+ T-cell activation, differentiation, and function. Notably, Yap-regulated gene expression signatures from TILs correlated with T-cell infiltration and patient survival across multiple human cancers in The Cancer Genome Atlas (TCGA), including melanoma and lung cancer and identifies Yap as a broad suppressor of CD4+ and CD8+ T-cell activation and function and a key regulator of T-cell tumor infiltration and survival in cancer immunotherapy patients. YAP deletion also increased the number of activated effector memory and resident memory T cells in response to both bacterial and viral infections.

T cell activation occurs through simultaneous engagement of the T cell receptor and co-stimulatory molecules (i.e., CD3 and CD28). This results in the activation of downstream signaling pathways (e.g., PI3K signaling), and eventual immune response (involving cytokine production). Following activation, a T cell expresses a variety of proteins (also known as markers), including, but not limited to CD44, CD69, CD71, CD25, and HLA-DR. In addition, an activated T cell has an altered cell surface protein glycosylation profile.

T cells, a type of lymphocyte, i.e., a cell which develops in the thymus gland, plays a central role in the immune response. T cells can be distinguished from other lymphocytes by the presence of a T-cell receptor on the cell surface. These immune cells originate as precursor cells, derived from bone marrow, and develop into several distinct types of T cells once they have migrated to the thymus gland. T cell differentiation continues even after they have left the thymus.

Helper CD4+ T cells, also known as T helper cells, or TH cells, assist other lymphocytes, including maturation of B cells into plasma cells and memory B cells, and activation of cytotoxic T cells and macrophages. These cells express the CD4 on their surfaces. Helper T cells become activated when they are presented with peptide antigens by MHC class II molecules, which are expressed on the surface of antigen-presenting cells (APCs). Once activated, they divide rapidly and secrete cytokines that regulate or assist the immune response. These cells can differentiate into one of several subtypes, which have different roles. Cytokines direct T cells into particular subtypes, for example, Th1 cells (which produce IFNγ), Th2 cells (which produce IL-4), Th17 cells (which produce IL-17), Th9 cells (which produce IL-9), and TfH cells (which produce IL-21 and IL-4).

Cytotoxic CD8+ T cells, also known as TC cells, CTLs, T-killer cells, or killer T cells, function to kill microbial-infected cells and tumor cells. Cytotoxic CD8+ T cells are defined by the expression of CD8+ on the cell surface, and recognize their targets by binding to short peptides (8-11AA) associated with MHC class I molecules, present on the surface of all nucleated cells. CD8+ T cells also produce the key cytokines IL-2 and IFNγ, which influence the effector functions of other cells, in particular macrophages and NK cells.

Naïve T cells, also known as antigen-naïve T cells, can expand and differentiate into memory and effector T cells, when contacted by a cognate antigen within the context of an MHC molecule on the surface of a professional antigen presenting cell (e.g. a dendritic cell), and appropriate co-stimulation is present. memory T cell subtypes is that they are long-lived and can quickly expand to large numbers of effector T cells upon re-exposure to their cognate antigen. Populations of memory T cells haven been shown, including tissue-resident memory T (Trm) cells, stem memory TSCM cells, and virtual memory T cells. Memory T cells may be either CD4+ or CD8+ and usually express CD45RO.

Central memory T cells (TCM cells), found in the lymph nodes and in the peripheral circulation, express CD45RO, C—C chemokine receptor type 7 (CCR7), L-selectin (CD62L), and have intermediate to high expression of CD44. This memory subpopulation is commonly found in the lymph nodes and in the peripheral circulation. Effector memory T cells (TEM cells) express CD45RO, and have intermediate to high expression of CD44, and lack expression of CCR7 and L-selectin. These memory T cells lack lymph node-homing receptors and are thus found in the peripheral circulation and tissues. Terminally differentiated effector memory (TERMA) cells re-expressing CD45RA, which is a marker usually found on naïve T cells. Tissue resident memory T cells (TRM) occupy tissues (e.g., skin, lung, etc.) without recirculating and express the cell surface marker αeβ7, also known as CD103.

Regulatory CD4+ T cells are crucial for the maintenance of immunological tolerance by shutting down T cell-mediated immunity toward the end of an immune reaction and to suppress autoreactive T cells that escaped the process of negative selection in the thymus. There are two major classes of CD4+ Treg cells—FOXP3+ Treg cells and FOXP3− Treg cells. Regulatory T cells can develop either during normal development in the thymus, and are then known as thymic Treg cells, naturally occuring, or adaptive, or can be induced peripherally and are called peripherally derived Treg cells or induced. Both subsets require the expression of the transcription factor FOXP3 which can be used to identify the cells. Several other types of T cell have suppressive activity, but do not express FOXP3, such as Tr1 cells, which express IL-10, Th3 cells, which express TGF-beta, and Treg17 cells.

Natural killer T cell bridge the adaptive immune system with the innate immune system. Unlike conventional T cells that recognize peptide antigens presented by major histocompatibility complex (MHC) molecules, NKT cells recognize glycolipid antigen presented by CD1d. Once activated, these cells can perform functions ascribed to both Th and Tc cells (i.e., cytokine production and release of cytolytic/cell killing molecules). They are also able to recognize and eliminate some tumor cells and cells infected with, e.g., a herpes virus.

Gamma delta T cells (γδ T cells) represent a small subset of T cells which possess a γδ TCR rather than the a TCR on the cell surface. The majority of T cells express a TCR chains. This group of T cells is much less common in mammals, e.g., humans and mice, representing about 2% of total T cells, and are found mostly in the gut mucosa, within a population of intraepithelial lymphocytes. While the antigenic molecules that activate γδ T cells are mostly unknown, γδ T cells are not MHC-restricted and seem to be able to recognize whole proteins rather than requiring peptides to be presented by MHC molecules on APCs. Some murine γδ T cells recognize MHC class IB molecules. Human γδ T cells, which use the Vγ9 and Vδ2 gene fragments, constitute the major γδ T cell population in peripheral blood, and specifically and rapidly respond to a set of nonpeptidic phosphorylated isoprenoid precursors, collectively named phosphoantigens, which are produced by virtually all living cells. The most common phosphoantigens from animal and human cells (including cancer cells) are isopentenyl pyrophosphate (IPP) and its isomer dimethylallyl pyrophosphate (DMPP). Many microbes produce the highly active compound hydroxy-DMAPP (HMB-PP) and corresponding mononucleotide conjugates, in addition to IPP and DMAPP. Plant cells produce both types of phosphoantigens. Drugs activating human Vγ9/Vδ2 T cells comprise synthetic phosphoantigens and aminobisphosphonates, which upregulate endogenous IPP/DMAPP.

In various embodiment, the T cell used in the invention described herein is a naïve T cell, a cytotoxic T cell, a memory T cell, a natural killer T cell, a tumor infiltrating T cell, a regulatory T cell, a helper T cell, or a synthetic T cell. In various embodiments, the T cell is a αβ T cell, γδ T cell, CD8+ T cell, or a CD4+ T cell. Methods for identifying and isolating a particular type T cell are known in the art, e.g., assessing a T cell for a cell surface marker that distinguishes a type of T cells, e.g., as described herein above.

In one embodiment, the T cell is not a Treg. In one embodiment, the T cell is not a cell derived from a T reg. In one embodiment, the T cell is not a Treg or a cell derived from a T reg.

In one embodiment, the T cell is not a CD4+ cell. In one embodiment, the T cell is not a cell derived from a CD4+ cell. In one embodiment, the T cell is not a CD4+ cell or a cell derived from a CD4+ cell.

In one embodiment, the T cell is not a Treg or CD4+ cell. In one embodiment, the T cell is not a cell derived from a Treg or CD4+ cell. In one embodiment, the T cell is not a Treg or CD4+ cell, or a cell derived from a Treg or CD4+ cell.

Provided herein is a method for activating a T cell comprising contacting a T cell with a pharmaceutically effective amount of an agent that inhibits Yap for a time sufficient, and under conditions to induce activation the T cell. In one embodiment, contacting the population of T cells with an agent that inhibits Yap results in the activation of the T cell population. In one embodiment, following contact by an agent, at least 5% of the T cell population will be activated. In another embodiment, following contact by an agent, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99% or more will be activated. In one embodiment, contacting a population of T cells with an agent that inhibits Yap increases the population of activated T cells by at least 5% as compared to an appropriate control. In one embodiment, contacting a population of T cells with an agent that inhibits Yap increases the population of activated T cells by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99% or more as compared to an appropriate control. As used herein, an appropriate control refers to a substantially similar population of T cells that is not contacted by an agent that inhibits Yap. One skilled in the art will be capable of determining if a T cell has become activated following contact with an agent described herein. For example, one can use a Proliferative Capacity assay (CFSE dilution and absolute T cell numbers are assessed by FACS using fluorescently-labeled counting beads), a Cytokine Production assay (10-Plex Luminex Assays using cytokine levels as a readout), a Target-cell Killing Capacity assay (Bioluminescence analysis of target cells in vitro or animal model system to track both tumor and T cells infused in immunodeficient mice), and/or a Cell Degranulation Analysis (CD107a release assay in response to target cells as measured by FACS). One skilled in the art can additionally determine if a T cell is activated by assessing the markers present on the T cell surface (e.g., CD44, CD69, CD71, CD25, and HLA-DR), or by examining the glycosylation profile of the cell surface.

Further provided herein is a method for differentiating a T cell comprising contacting a T cell with a pharmaceutically effective amount an agent that inhibits Yap for a time, and under conditions sufficient to induce differentiation of the T cell. In one embodiment, contacting the population of T cells and with an agent that inhibits Yap results in the differentiation of the T cell population. For example, a CD4+ T cell can be differentiated into a Th1, Th2, Th17, or Treg cell. In one embodiment, contacting the population of CD4+ T cells with an agent that inhibits under Th1, Th2, Th17, or Treg differentiating conditions results in the enhanced differentiation of the CD4+ T cell population. In one embodiment, following contact by an agent, at least 5% of the T cell population will become differentiated. In another embodiment, following contact by an agent, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99% or more of the T cell population will become differentiated. In one embodiment, contacting a population of T cells with an agent that inhibits Yap increases the population of differentiated T cells by at least 5% as compared to an appropriate control. In one embodiment, contacting a population of T cells with an agent that inhibits Yap increases the population of differentiated T cells by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99% or more, or at least 2×, 3×, 4×, 5×, 6×, 7×, 8×, 9×, 10×, or more as compared to an appropriate control. As used herein, an appropriate control refers to a substantially similar population of T cells that is not contacted by an agent that inhibits Yap. One skilled in the art will be capable of determining if a T cell has become differentiated following contact with an agent described herein, for example, assessing the marker profile of the differentiated T cell surface, e.g., expressed by the T cell and/or on the cell surface of the T cell. For example, when differentiated from a CD4+ T cell, the Th1 cells express T-BET and IFNγ, Th2 cells express GATA3 and IL-4, Th17 cells express RORγ and IL-17, or Treg cells express Foxp3.

In one embodiment, an agent that inhibits Yap is used enhance Treg differentiation; Tregs have immunosuppressive properties. These differentiated Tregs are can be used, e.g., in a T cell transplantation to treat, e.g., an autoimmune disease.

Further provided herein is a method of for activating or differentiating a T cell comprising (a) providing a population of T cells, (b) contacting the T cell population with an agent that inhibits YAP, and (c) ex vivo culturing for a time, and under conditions sufficient to induce activation or differentiation of the T cell population

In various embodiments, prior to contacting, the T cell is obtained from a biological source. As used herein “biological source” can be a living source, e.g., a mammal, preferably a human, such as a donor or patient, or an artificial T cells obtained from a lab or manufacturing setting. Artificial T cells are further described, e.g., Hasani-Sadrabadi, M M, et al. Advanced Materials (2018), which is incorporated herein by reference in its entirety. In aspects described herein, T cells to be contacted with an agent that inhibits Yap can be obtained using standard techniques known in the field. For example, T cells can be isolated from peripheral blood taken from a donor or patient. T cells can be isolated from a mammal. Preferably, T cells are isolated from a human. The biological source can be a healthy patient, that is, a patient that has not been diagnosed with or at risk of having a disease or disorder, e.g., cancer, an auto-immune disease, or a microbial infectious disease. Alternatively, the biological source can be a patient having been diagnosed with a disease or disorder, e.g., cancer, an auto-immune disease, or a microbial infectious disease, or at risk of having a disease or disorder.

In one embodiment, the contacting of a population of T cell occurs in vitro. In one embodiment, the contacting of a population of T cell occurs in vivo. In one embodiment, the contacting of a population of T cell occurs ex vivo. In another embodiment, the contacting occurs in suspension. In one embodiment, a population of T cells is contacted with an agent for at least 1 day, at least 2 days, at least 3 days, at least 4 days, for at least 5 days, for at least 6 days, for at least 7 days, for at least 8 days, for at least 9 days, for at least 10 days, for at least 11 days, for at least 12 days, for at least 13 days, for at least 14 days, for at least 15 days, for at least 16 days, for at least 17 days, for at least 18 days, for at least 19 days, for at least 20 days, for at least 21 days, for at least 22 days, for at least 23 days, for at least 24 days, for at least 25 days or more, or for at least 2-25 day, at least 5-25 days, at least 10-25 days, at least 15-25 days, at least 20-25 days, at least 2-20 days, at least 2-15 days, at least 2-10 days, at least 2-5 days, at least 5-20 days, at least 10-20 days, at least 15-20 days, at least 5-10 days, at least 5-15 days, or any range therein. In a preferred embodiment of any aspect, the contact of the population of T cells and agent is continuous for the duration of the contact.

In one embodiment, contacting the population of T cells with an agent that inhibits Yap results in an increased capacity for the T cell population to infiltrate a tumor microenvironment and/or tumor. In one embodiment, contacting a population of T cells with an agent that inhibits Yap increases the capacity for the T cell population to infiltrate a tumor microenvironment and/or tumor by at least 5% as compared to an appropriate control. In one embodiment, contacting a population of T cells with an agent that inhibits Yap increases the capacity for the T cell population to infiltrate a tumor microenvironment and/or tumor by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99% or more, or at least 2×, 3×, 4×, 5×, 6×, 7×, 8×, 9×, 10×, 50×, 100×, 150×, 200×, 250×, 300×, 350×, 500×, 750× or more as compared to an appropriate control. As used herein, an appropriate control refers to a substantially similar population of T cells that is not contacted by an agent that inhibits Yap. One skilled in the art will be capable of determining if a T cell has an increased capacity for the T cell population to infiltrate a tumor microenvironment and/or tumor, e.g., Target-cell Killing Capacity assay (Bioluminescence analysis of target cells in vitro or animal model system to track both tumor and T cells infused in immunodeficient mice). Microscopy of tumor sections using antibodies specific for T cell markers, or FACS analysis of isolated and dissociated tumors using antibodies specific for T cell markers can further be used to assess if a T cell has infiltrated the tumor microenvironment.

In one embodiment, contacting the population of T cells with an agent that inhibits Yap results in an increased expression of chemokine or chemokine receptor genes on the T cell, for example, those chemokines or chemokine receptors listed in Table 3 herein. In one embodiment, contacting the population of T cells with an agent that inhibits Yap results in an increased expression of at least one gene listed in Table 3 herein. In one embodiment, contacting a population of T cells with an agent that inhibits Yap increases the expression of chemokine or chemokine receptor genes, or any gene listed in Table 3, in the T cell by at least 5% as compared to an appropriate control. In one embodiment, contacting a population of T cells with an agent that inhibits Yap increases the expression of chemokine or chemokine receptor genes, or any gene listed in Table 3, in the T cell by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99% or more, or at least 2×, 3×, 4×, 5×, 6×, 7×, 8×, 9×, Ox, 50×, 100×, 150×, 200×, 250×, 300×, 350×, 500×, 750× or more as compared to an appropriate control. As used herein, an appropriate control refers to a substantially similar population of T cells that is not contacted by an agent that inhibits Yap. One skilled in the art will be capable of determining if a T cell has an increased expression of chemokine or chemokine receptor genes, or any gene listed in Table 3, in the T cell, e.g., by measuring the mRNA or gene product (protein) level of the chemokine or chemokine receptor genes using standard techniques, e.g., PCR-based assays or western blotting, respectively. Imaging of cell surface markers on the T cell (either known or described herein) can reveal whether a T cell has infiltrated the tumor microenvironment and/or tumor.

In one embodiment, contacting the population of T cells with an agent that inhibits Yap results in an increased expression of TEAD1, TEAD2, TEAD3 and/or TEAD4 protein in the T cell. For example, following contact with an agent that inhibits Yap, a CD8+ T cell has increased TEAD1 expression, and a CD4+ cell has increased TEAD1 and TEAD3 expression. In one embodiment, contacting a population of T cells with an agent that inhibits Yap increases the expression of TEAD1, TEAD2, and/or TEAD3 protein in the T cell by at least 5% as compared to an appropriate control. In one embodiment, contacting a population of T cells with an agent that inhibits Yap increases the expression of TEAD1, TEAD2, and/or TEAD3 protein in the T cell by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99% or more, or at least 2×, 3×, 4×, 5×, 6×, 7×, 8×, 9×, 10×, 50×, 100×, 150×, 200×, 250×, 300×, 350×, 500×, 750× or more as compared to an appropriate control. As used herein, an appropriate control refers to a substantially similar population of T cells that is not contacted by an agent that inhibits Yap. One skilled in the art will be capable of determining if a T cell has an increased TEAD1, TEAD2, TEAD3 and/or TEAD4 protein in the T cell, e.g., by measuring the mRNA or protein level of the TEAD1, TEAD2, TEAD3 and/or TEAD4 using standard techniques, e.g., PCR-based assays or western blotting, respectively.

In one embodiment, contacting the population of T cells with an agent that inhibits Yap results in an increased expression of WWTR1/Taz protein in the T cell. In one embodiment, contacting a population of T cells with an agent that inhibits Yap increases the expression of WWTR/Taz protein in the T cell by at least 5% as compared to an appropriate control. In one embodiment, contacting a population of T cells with an agent that inhibits Yap increases the expression of WWTR/Taz protein in the T cell by at least 10, at least 15, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99% or more, or at least 2×, 3×, 4×, 5×, 6×, 7×, 8×, 9×, 10×, 50×, 100×, 150×, 200×, 250×, 300×, 350×, 500×, 750× or more as compared to an appropriate control. As used herein, an appropriate control refers to a substantially similar population of T cells that is not contacted by an agent that inhibits Yap. One skilled in the art will be capable of determining if a T cell has an increased WWTR1/Taz protein in the T cell, e.g., by measuring the mRNA or protein level of WWTR1/Taz using standard techniques, e.g., PCR-based assays or western blotting, respectively.

Accordingly, in one embodiment, the methods described herein further comprise contacting the T cell population with an agent that inhibits WWTR1/Taz. The agent that inhibits Yap and the agent that inhibits WWTR1/Taz can contact the population of T cells at substantially the same, or at different times, e.g., a first agent is removed from contacting a population of T cells prior to the second agent contacting the population of T cells. Alternatively, the contacting with the agent that inhibits Yap and the agent that inhibits WWTR1/Taz can overlap for any period of time during contacting. Further, the order in which the T cell population is contacted by an agent that inhibits Yap and the agent that inhibits WWTR1/Taz can be in any order sufficient to induce activation or differentiation of a T cell, e.g., the T cell population can be contacted with the agent that inhibits Yap first, or following contact with the agent that inhibits WWTR1/Taz.

In one embodiment, contacting the population of T cells and with an agent that inhibits Yap does not alter the proliferation rate, e.g., does not increase or decrease the proliferation rate, of the population of T cells.

In one embodiment, contacting the population of T cells and with an agent that inhibits Yap does not alter, e.g., increase or decrease the rate of, the maturation of the T cell in the thymus or the output of T cells from the thymus.

In certain embodiments, the methods described herein further comprise the step of, after culturing, transplanting the population of contacted T cells into a recipient subject. Transplanting a population of contacted T cells is further described in detail herein below.

In one embodiment, the T cell has previously been genetically modified, e.g., to alter the genetic material to add or delete nucleic acid sequence, or engineered, e.g., to comprise a chimeric antigen receptor (CAR) prior to contacting with an agent that inhibits Yap. In one embodiment, an activated or differentiated T cell produced using methods described herein can further by genetically modified, e.g., to alter the genetic material to add or delete nucleic acid sequence, or engineered, e.g., to comprise a chimeric antigen receptor (CAR). “Chimeric antigen receptor” or “CAR” or “CARs” as used herein refer to engineered receptors, which graft an antigen specificity onto cells (for example T cells such as naïve T cells, central memory T cells, effector memory T cells or combination thereof). CARs are also known as artificial T-cell receptors, chimeric T-cell receptors or chimeric immunoreceptors. In one embodiment, the CARs useful in the technology described herein comprise at least two antigen-specific targeting regions, an extracellular domain, a transmembrane domain, and an intracellular signaling domain. In such embodiments, the two or more antigen-specific targeting regions target at least two different antigens and may be arranged in tandem and separated by linker sequences. In another embodiment, the CAR until a sufficient number of cells is obtained for adoptive transfer to the patient. In this embodiment, the T cell is contacted with an agent described herein upon thawing.

As used herein, a “CAR T cell” refers to a T cell which expresses a CAR. When expressed in a T cell, CARs have the ability to redirect T-cell specificity and reactivity toward a selected target in a non-MHC-restricted manner, exploiting the antigen-binding properties of monoclonal antibodies. The non-MHC-restricted antigen recognition gives T-cells expressing CARs the ability to recognize an antigen independent of antigen processing, thus bypassing a major mechanism of tumor escape.

In one embodiment, the CAR's extracellular binding domain is composed of a single chain Fv fragment (scFv) derived from fusing the variable heavy and light regions of a monoclonal antibody. Alternatively, scFvs may be used that are derived from Fab's (instead of from an antibody, e.g., obtained from Fab libraries), in various embodiments, this scFv is fused to a transmembrane domain and then to an intracellular signaling domain. “First-generation” CARs include those that solely provide CD3zeta (CD3ζ) signals upon antigen binding, “Second-generation” CARs include those that provide both costimulation (e.g., CD28 or CD 137) and activation (CD3ζ). “Third-generation” CARs include those that provide multiple costimulatory (e.g., CD28 and CD 137) domains and activation domains (e.g., CD3ζ). In various embodiments, the CAR is selected to have high affinity or avidity for the antigen. A more detailed description of CARs and CAR T cells can be found in Maus et al. Blood 2014 123:2624-35; Reardon et al. Neuro-Oncology 2014 16:1441-1458; Hoyos et al. Haematologica 2012 97:1622; Byrd et al. J Clin Oncol 2014 32:3039-47; Maher et al. Cancer Res 2009 69:4559-4562; and Tamada et al. Clin Cancer Res 2012 18:6436-6445; each of which is incorporated by reference herein in its entirety. An ordinarily skilled person can generate CAR T cells to be contacted with an aAPC using standard techniques.

An aspect described herein further provide an activated T cell produced using any method described herein.

An aspect described herein further provide a differentiated T cell produced using any method described herein.

Another aspect provided herein is a method to deactivate an activated T cell, comprising contacting the T cell with an agent that increases the expression of Yap, for example, a vector comprising an expression plasmid of Yap, an activator of Yap, or alternatively, an inhibitor of a gene or protein that represses Yap. One skilled in the art can identify such agent.

In one other embodiment, following contact with an agent that increases Yap expression, Yap expression in T cells is increased by at least by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99% or more, or at least 2×, 3×, 4×, 5×, 6×, 7×, 8×, 9×, 10×, 50×, 100×, 150×, 200×, 250×, 300×, 350×, 500×, 750× or more as compared to reference level. As used herein, a reference level refers to the level of Yap in an activated T cells, or the T cell prior to contact. One skilled in the art will be capable of determining the level of Yap in a T cell e.g., by measuring the mRNA or protein level of Yap using standard techniques, e.g., PCR-based assays or western blotting, respectively.

### Agents

Various aspects required that a population of T cells is contacted with an agent that inhibits Yap, or administering an agent that inhibits Yap to a subject having, or at risk of having a disease or disorder caused by or associated with T cell dysfunction. n various embodiments, an agent that inhibits WWTR1/Taz is further use to contact the population of T cell or administered to the subject. In one embodiment, the agent that inhibits, e.g., Yap or WWTR1/Taz, is a small molecule, an antibody or antibody fragment, a peptide, protein, e.g. a ligand, an antisense oligonucleotide, a genome editing system, or an RNAi.

An agent is considered effective for inhibiting e.g., Yap or WWTR1/Taz if, for example, upon administration, it inhibits the presence, amount, activity and/or level of Yap in the cell, for example, a T cell.

An agent can inhibit e.g., the transcription, or the translation of Yap or WWTR1/Taz in the cell. An agent can inhibit the activity or alter the activity (e.g., such that the activity no longer occurs, or occurs at a reduced rate) of, e.g., Yap in the cell (e.g., Yap's expression).

In one embodiment, an agent that inhibits, e.g., Yap or WWTR/Taz, inhibits the expression level or activity of Yap. To determine is an agent is effective at inhibiting Yap, mRNA and protein levels of a given target (e.g., Yap) can be assessed using RT-PCR and western-blotting, respectively. Biological assays that detect the activity of Yap can be used to assess if Yap is inhibited, for example, to determine if an increase in T cell activation, as seen by an expression of, e.g., CD44, CD69, CD71, CD25, and/or HLA-DR, has occurred.

In one embodiment, an agent that inhibits the level and/or activity of Yap by at least 5%, by at least 10%, by at least 15%, by at least 20%, by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, by at least 95%, by at least 99% or more as compared to an appropriate control. As used herein, an “appropriate control” refers to the level and/or activity of Yap prior to administration of the agent, or the level and/or activity of Yap in a population of cells, e.g., T cells, that was not in contact with the agent.

In one embodiment, an agent that inhibits the level and/or activity of WWTR1/Taz by at least 5%, by at least 10%, by at least 15%, by at least 20%, by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, by at least 95%, by at least 99% or more as compared to an appropriate control. As used herein, an “appropriate control” refers to the level and/or activity of WWTR1/Taz prior to administration of the agent, or the level and/or activity of WWTR1/Taz in a population of cells, e.g., T cells, that was not in contact with the agent.

The agent may function directly in the form in which it is administered. Alternatively, the agent can be modified or utilized intracellularly to produce something which inhibits, e.g., Yap or WWTR1/Taz, such as introduction of a nucleic acid sequence into the cell and its transcription resulting in the production of the nucleic acid and/or protein inhibitor of Yap. In some embodiments, the agent is any chemical, entity or moiety, including without limitation synthetic and naturally-occurring non-proteinaceous entities. In certain embodiments the agent is a small molecule having a chemical moiety. For example, chemical moieties included unsubstituted or substituted alkyl, aromatic, or heterocyclyl moieties including macrolides, leptomycins and related natural products or analogues thereof. Agents can be known to have a desired activity and/or property, or can be identified from a library of diverse compounds.

In one embodiment, the agent further comprises a moiety for targeting a T cell. In one embodiment, the moiety is a T cell target molecule. As used herein, a “T cell target molecule” is a ligand for a T cell receptor, e.g., a naturally-occurring T cell receptor, or an engineered T cell receptor, e.g., a CAR. A T cell target molecule will activate a T cell, provided appropriate co-stimulatory signals. In this context, the T cell target molecule can be a full length target polypeptide, i.e., as it occurs in vivo or native to the genome, or a fragment thereof that binds the T cell receptor and promotes proliferation and/or activation of the T cell (given the appropriate co-stimulatory signals).

In one embodiment, the T cell target molecule coupled to an agent includes, but is not limited to CD19, BCMA, SLAMF7, EGFR or EGFR variant III. In one embodiment, the expression of a T cell target coupled to the agent will facilitate the recognition of the agent by a T cell or CAR T cell. In this embodiment, the presence of the T cell target molecule on the agent provides a mechanism, e.g., for inhibiting Yap specifically in a T cell.

In one embodiment, the T cell-specific cell surface polypeptide a polypeptide present on the surface of the T cell, for example, CD4 or CD8.

In various embodiments, the agent is a small molecule that inhibits, e.g., Yap or WWTR1/Taz. Methods for screening small molecules are known in the art and can be used to identify a small molecule that is efficient at, for example, increasing T cell activation in a population of T cells, as seen by an expression of, e.g., CD44, CD69, CD71, CD25, and/or HLA-DR, given the desired target (e.g., Yap).

Exemplary small molecules that target Yap for inhibition include, e.g., Verteporfin or YAP/TAZ inhibitor-1. Verteporfin (trade name Visudyne), a benzoporphyrin derivative, is a medication used as a photosensitizer for photodynamic therapy to eliminate the abnormal blood vessels in the eye associated with conditions such as the wet form of macular degeneration. Following administration, a laser treatment can be used to activate the small molecule.

The chemical formula of Verteporfin is C₄₁H₄₂N₄O₈. Verteporfin (CAS No: 129497-78-5) is commercially available, and has a known chemical of

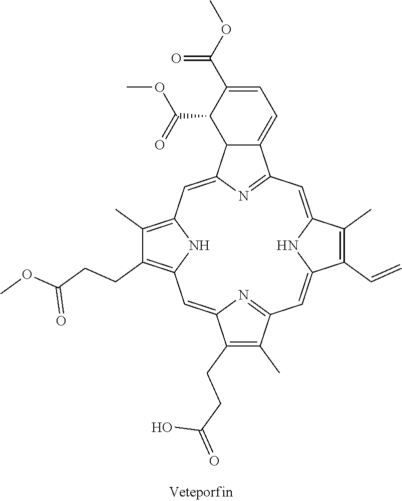 [SMILES: C=Cc1c(C)c2cc3nc(cc4[nH]c(cc5nc(cc1[nH]2)C(C)=C5CCC(=O)O)c(CCC(=O)OC)c4C)C1C3=CC=C(C(=O)OC)[C@H]1C(=O)OC]

YAP/TAZ inhibitor-1 is a small molecule dual inhibitor of Yap and Taz, and is further described in, e.g., International Application No. WO2017058716A1, which is incorporated herein by reference in its entirety.

The chemical formula of YAP/TAZ inhibitor-1 is C₃₃H₃₉N₃O₅S₂. YAP/TAZ inhibitor-1 (CAS No: 2093565-23-0) is commercially available, and has a known chemical of

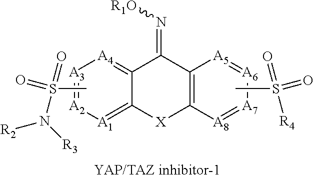

In one embodiment, the agent that inhibits Yap is a statin. In one embodiment, the agent that inhibits YAP antagonist comprises simvastatin. In another aspect, exemplary YAP antagonists include β-adrenergic receptor agonists, Dobutamine, Latrunculin A, Latrunculin B, cytochalasin D, actin inhibitors, drugs that act on the cytoskeleton, Blebbistatitin, Botulinum toxin C3, and RHO kinase-targeting drugs (e.g., Y27632).

In various embodiments, the agent that inhibits, e.g., Yap or WWTR1/Taz is an antibody or antigen-binding fragment thereof, or an antibody reagent that is specific for Yap. The term “antibody” or “antibody reagent” broadly refers to any immunoglobulin (Ig) molecule or compositions of Igs and/or immunologically active portions of immunoglobulin molecules (i.e., molecules that contain an antigen binding site that immunospecifically bind an antigen) comprised of four polypeptide chains, two heavy (H) chains and two light (L) chains, or any functional fragment, mutant, variant, or derivation thereof, which retains the essential epitope binding features of an Ig molecule. Such mutant, variant, or derivative antibody formats are known in the art. Nonlimiting embodiments of which are discussed below, and include but are not limited to a variety of forms, including full length antibodies and antigen-binding portions thereof; including, for example, an immunoglobulin molecule, a monoclonal antibody, a chimeric antibody, a CDR-grafted antibody, a human antibody, a humanized antibody, a single chain antibody, a Fab, a F(ab′), a F(ab′)2, a Fv antibody, fragments produced by a Fab expression library, a disulfide linked Fv, a scFv, a single domain antibody (dAb), a diabody, a multispecific antibody, a dual specific antibody, an anti-idiotypic antibody, a bispecific antibody, a functionally active epitope-binding fragment thereof, bifunctional hybrid antibodies (e.g., Lanzavecchia et al., Eur. J. Immunol. 17, 105 (1987)) and single chains (e.g., Huston et al., Proc. Natl. Acad. Sci. U.S.A., 85, 5879-5883 (1988) and Bird et al., Science 242, 423-426 (1988), which are incorporated herein by reference) and/or antigen-binding fragments of any of the above (See, generally, Hood et al., Immunology, Benjamin, N.Y., 2ND ed. (1984), Harlow and Lane, Antibodies. A Laboratory Manual, Cold Spring Harbor Laboratory (1988) and Hunkapiller and Hood, Nature, 323, 15-16 (1986), which are incorporated herein by reference). Antibodies also refer to immunoglobulin molecules and immunologically active portions of immunoglobulin molecules, i.e., molecules that contain antigen or target binding sites or “antigen-binding fragments.” The antibody or immunoglobulin molecules described herein can be of any type (e.g., IgG, IgE, IgM, IgD, IgA and IgY), class (e.g., IgG1, IgG2, IgG3, IgG4, IgA1 and IgA2) or subclass of immunoglobulin molecule, as is understood by one of skill in the art. Furthermore, in humans, the light chain can be a kappa chain or a lambda chain.

In a full-length antibody, each heavy chain is comprised of a heavy chain variable domain (abbreviated herein as HCVR or VH) and a heavy chain constant region. The heavy chain constant region is comprised of three domains: CH1, CH2, and CH3. Each light chain is comprised of a light chain variable domain (abbreviated herein LCVR as VL) and a light chain constant region. The light chain constant region is comprised of one domain, CL. The VH and VL regions can be further subdivided into regions of hypervariability, termed complementarity determining regions (CDRs), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. This structure is well-known to those skilled in the art. The chains are usually linked to one another via disulfide bonds.

The term “Fc region” is used to define the C-terminal region of an immunoglobulin heavy chain, which may be generated by papain digestion of an intact antibody. The Fc region may be a native sequence Fc region or a variant Fc region. The Fc region of an immunoglobulin generally comprises two constant domains, a CH2 domain, and a CH3 domain, and optionally comprises a CH4 domain. Replacements of amino acid residues in the Fc portion to alter antibody effector function are known in the art (U.S. Pat. Nos. 5,648,260 and 5,624,821). The Fc portion of an antibody mediates several important effector functions, for example, cytokine induction, ADCC, phagocytosis, complement dependent cytotoxicity (CDC), and half-life/clearance rate of antibody and antigen-antibody complexes. In some cases, these effector functions are desirable for therapeutic antibody but in other cases might be unnecessary or even deleterious, depending on the therapeutic objectives. Certain human IgG isotypes, particularly IgG1 and IgG3, mediate ADCC and CDC via binding to Fc.gamma.Rs and complement C1q, respectively. Neonatal Fc receptors (FcRn) are the critical components determining the circulating half-life of antibodies. In still another embodiment at least one amino acid residue is replaced in the constant region of the antibody, for example the Fc region of the antibody, such that effector functions of the antibody are altered.

The term “antigen-binding portion” of an antibody refers to one or more fragments of an antibody that retain the ability to specifically bind to an antigen (e.g., CD80 or CD86). Antigen-binding functions of an antibody can be performed by fragments of a full-length antibody. Such antibody fragment embodiments may also be incorporated in bispecific, dual specific, or multi-specific formats such as a dual variable domain (DVD-Ig) format; specifically binding to two or more different antigens. Examples of binding fragments encompassed within the term “antigen-binding portion” of an antibody include (i) a Fab fragment, a monovalent fragment consisting of the VL, VH, CL, and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al. (1989) Nature, 341: 544-546; PCT Publication No. WO 90/05144), which comprises a single variable domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see, for example, Bird et al. (1988) Science 242: 423-426; and Huston et al. (1988) Proc. Natl. Acad. Sci. USA 85: 5879-5883). Such single chain antibodies are also intended to be encompassed within the term “antigen-binding portion” of an antibody.

Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see, for example, Holliger et al. (1993) Proc. Natl. Acad. Sci. USA 90: 6444-6448; Poljak (1994) Structure 2: 1121-1123); Kontermann and Dubel eds., Antibody Engineering, Springer-Verlag, N.Y. (2001), p. 790 (ISBN 3-540-41354-5). As used herein, a “bispecific antibody” refers to an antibody that can simultaneously bind to two different types of antigen.

In addition, single chain antibodies also include “linear antibodies” comprising a pair of tandem Fv segments (VH-CH1-VH-CH1) which, together with complementary light chain polypeptides, form a pair of antigen binding regions (Zapata et al. (1995) Protein Eng. 8(10): 1057-1062; and U.S. Pat. No. 5,641,870). An immunoglobulin constant (C) domain refers to a heavy (CH) or light (CL) chain constant domain. Murine and human IgG heavy chain and light chain constant domain amino acid sequences are known in the art.

In some embodiments of any of the aspects, the antibody reagent is a human or humanized antibody.

The term “humanized antibody” refers to antibodies that comprise heavy and light chain variable domain sequences from a non-human species (e.g., a mouse) but in which at least a portion of the VH and/or VL sequence has been altered to be more “human-like”, i.e., more similar to human germline variable sequences. Accordingly, “humanized” antibodies are a form of a chimeric antibody, that are engineered or designed to comprise minimal sequence derived from non-human immunoglobulin. For the most part, humanized antibodies are human immunoglobulins (recipient or acceptor antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, Fv framework region (FR) residues of the human immunoglobulin are replaced by corresponding non-human residues. Furthermore, humanized antibodies can comprise residues which are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992). As used herein, a “composite human antibody” or “deimmunized antibody” are specific types of engineered or humanized antibodies designed to reduce or eliminate T cell epitopes from the variable domains.

One type of humanized antibody is a CDR-grafted antibody, in which human CDR sequences are introduced into non-human VH and VL sequences to replace the corresponding nonhuman CDR sequences. Also “humanized antibody” is an antibody or a variant, derivative, analog or fragment thereof which immunospecifically binds to an antigen of interest and which comprises a framework (FR) region having substantially the amino acid sequence of a human antibody and a complementary determining region (CDR) having substantially the amino acid sequence of a non-human antibody. As used herein, the term “substantially” in the context of a CDR refers to a CDR having an amino acid sequence at least 80%, at least 85%, at least 90%, at least 95%, at least 98% or at least 99% identical to the amino acid sequence of a non-human antibody CDR. A humanized antibody comprises substantially all of at least one, and typically two, variable domains (Fab, Fab′, F(ab′). sub.2, FabC, Fv) in which all or substantially all of the CDR regions correspond to those of a non-human immunoglobulin (i.e., donor antibody) and all or substantially all of the framework regions are those of a human immunoglobulin consensus sequence. In an embodiment, a humanized antibody also comprises at least a portion of an immunoglobulin constant region (Fc), typically that of a human immunoglobulin. In some embodiments of any of the aspects, a humanized antibody contains both the light chain as well as at least the variable domain of a heavy chain. The antibody also may include the CH1, hinge, CH2, CH3, and CH4 regions of the heavy chain. In some embodiments of any of the aspects, a humanized antibody only contains a humanized light chain. In some embodiments of any of the aspects, a humanized antibody only contains a humanized heavy chain. In specific embodiments, a humanized antibody only contains a humanized variable domain of a light chain and/or humanized heavy chain. A humanized antibody may be selected from any class of immunoglobulins, including IgM, IgG, IgD, IgA and IgE, and any isotype including without limitation IgG1, IgG2, IgG3, and IgG4. The humanized antibody may comprise sequences from more than one class or isotype, and particular constant domains may be selected to optimize desired effector functions using techniques well known in the art.

The framework and CDR regions of a humanized antibody need not correspond precisely to the parental sequences, e.g., the donor antibody CDR or the consensus framework may be mutagenized by substitution, insertion and/or deletion of at least one amino acid residue so that the CDR or framework residue at that site does not correspond to either the donor antibody or the consensus framework. In an exemplary embodiment, such mutations, however, will not be extensive. Usually, at least 80%, preferably at least 85%, more preferably at least 90%, and most preferably at least 95% of the humanized antibody residues will correspond to those of the parental FR and CDR sequences. As used herein, the term “consensus framework” refers to the framework region in the consensus immunoglobulin sequence. As used herein, the term “consensus immunoglobulin sequence” refers to the sequence formed from the most frequently occurring amino acids (or nucleotides) in a family of related immunoglobulin sequences (see, e.g., Winnaker, From Genes to Clones (Verlagsgesellschaft, Weinheim, Germany 1987)). In a family of immunoglobulins, each position in the consensus sequence is occupied by the amino acid occurring most frequently at that position in the family. If two amino acids occur equally frequently, either can be included in the consensus sequence.

A “human antibody,” “non-engineered human antibody,” or “fully human antibody” is one which possesses an amino acid sequence which corresponds to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art. In one embodiment, the human antibody is selected from a phage library, where that phage library expresses human antibodies (Vaughan et al. Nature Biotechnology 14:309-314 (1996): Sheets et al. Proc. Natl. Acad. Sci. 95:6157-6162 (1998)); Hoogenboom and Winter, J. Mol. Biol., 227:381 (1991); Marks et al., J. Mol. Biol., 222:581 (1991)). Human antibodies can also be made by introducing human immunoglobulin loci into transgenic animals, e.g., mice in which the endogenous mouse immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10: 779-783 (1992); Lonberg et al., Nature 368: 856-859 (1994); Morrison, Nature 368:812-13 (1994); Fishwild et al., Nature Biotechnology 14: 845-51 (1996); Neuberger, Nature Biotechnology 14: 826 (1996); Lonberg and Huszar, Intern. Rev. Immunol. 13:65-93 (1995). Alternatively, the human antibody can be prepared via immortalization of human B lymphocytes producing an antibody directed against a target antigen (such B lymphocytes can be recovered from an individual or can have been immunized in vitro). See, e.g., Cole et al., Monoclonal Antibodies and Cancer Therapy, Alan R. Liss, p. 77 (1985); Boerner et al., J. Immunol., 147 (1):86-95 (1991); and U.S. Pat. No. 5,750,373.

In one embodiment, the antibody or antibody reagent binds to an amino acid sequence that corresponds to the amino acid sequence encoding Yap, isoform 1 (SEQ ID NO: 2).

(SEQ ID NO: 2)
MDPGQQPPPQPAPQGQGQPPSQPPQGQGPPSGPGQPAPAATQAA
PQAPPAGHQIVHVRGDSETDLEALFNAVMNPKTANVPQTVPMRLRKLPDS
FFKPPEPKSHSRQASTDAGTAGALTPQHVRAHSSPASLQLGAVSPGTLTP
TGVVSGPAATPTAQHLRQSSFEIPDDVPLPAGWEMAKTSSGQRYFLNHID
QTTTWQDPRKAMLSQMNVTAPTSPPVQQNMMNSASGPLPDGWEQAMTQDG
EIYYINHKNKTTSWLDPRLDPRFAMNQRISQSAPVKQPPPLAPQSPQGGV
MGGSNSNQQQQMRLQQLQMEKERLRLKQQELLRQAMRNINPSTANSPKCQ
ELALRSQLPTLEQDGGTQNPVSSPGMSQELRTMTTNSSDPFLNSGTYHSR
DESTDSGLSMSSYSVPRTPDDFLNSVDEMDTGDTINQSTLPSQQNRFPDY
LEAIPGTNVDLGTLEGDGMNIEGEELMPSLQEALSSDILNDMESVLAATK
LDKESFLTWL

In another embodiment, the anti-Yap antibody or antibody reagent binds to an amino acid sequence that comprises the sequence of SEQ ID NO: 2; or binds to an amino acid sequence that comprises a sequence with at least 80%, at least 85%, at least 90%, at least 95%, at least 96%, at least 97%, at least 98%, at least 99% or greater sequence identity to the sequence of SEQ ID NO: 2. In one embodiment, the anti-Yap antibody or antibody reagent binds to an amino acid sequence that comprises the entire sequence of SEQ ID NO: 2. In another embodiment, the antibody or antibody reagent binds to an amino acid sequence that comprises a fragment of the sequence of SEQ ID NO: 2, wherein the fragment is sufficient to bind its target, e.g., Yap, and for example, increasing T cell activation in a population of T cells, as seen by an expression of, e.g., CD44, CD69, CD71, CD25, and/or HLA-DR.

Anti-Yap antibodies are commercially available from, e.g., those antibodies listed in Table 1.

TABLE 1
Anti-YAP antibodies

| | Clone ID for | | |
|---|---|---|---|
| | monoclonal | Catalog | Reference |
| Target | antibodies | Numbers | Information |
| YAP | D8H1X | 14074 | Cell signaling |
| | | | Technologies |
| | | | (Danvers, MA, USA) |
| YAP/TAZ | D24E4 | 8418 | Cell signaling |
| | | | Technologies |
| YAP | 1A12 | 12395 | Cell signaling |
| | | | Technologies |
| YAP | | 4912 | Cell signaling |
| | | | Technologies |
| YAP | | NB110-58358 | Novus Biologicals |
| | | | (Centennial, |
| | | | Colorado, USA) |
| YAP | 2F12 | H00010413-M01 | Novus Biologicals |
| YAP | 867711 | MAB8094 | Novus Biologicals |
| YAP | 1A12 | NBP2-22117 | Novus Biologicals |
| YAP | SU33-06 | NBP2-67467 | Novus Biologicals |
| YAP | | NBP2-62669 | Novus Biologicals |
| YAP | | NBP1-46105 | Novus Biologicals |
| YAP | | Y4770 | Millipore Sigma, |
| | | | Burlington, |
| | | | Massachusetts, USA |
| YAP | 2H1 | SAB1404823 | Millipore Sigma |
| YAP | | AV50530 | Millipore Sigma |
| YAP | | HPA038885 | Millipore Sigma |
| YAP | | SAB2108066 | Millipore Sigma |
| YAP | 4D11.1 | MABC203 | Millipore Sigma |
| YAP | 4F22 | ZRB1001 | Millipore Sigma |
| YAP | EP1674Y | ab52771 | Abcam, Cambridge, |
| | | | United Kingdom |
| YAP | | ab56701 | Abcam |
| YAP | | ab81183 | Abcam |
| YAP | | ab226817 | Abcam |
| YAP | | ab245286 | Abcam |
| YAP | | ab246007 | Abcam |

In another embodiment, the agent that inhibits WWTR1/Taz is an anti-WWTR1/Taz antibody or antibody reagent. In one embodiment, the antibody or antibody reagent binds to an amino acid sequence that corresponds to the amino acid sequence encoding WWTR1, transcript variant 2 (SEQ ID NO: 4).

SEQ ID NO: 4 presents that nucleic acid sequence for WWTR1, transcript variant 2.

(SEQ ID NO: 4)
MNPASAPPPLPPPGQQVIHVTQDLDTDLEALFNSVMNPKPSSWRKKILPE
SFFKEPDSGSHSRQSSTDSSGGHPGPRLAGGAQHVRSHSSPASLQLGTGA
GAAGSPAQQHAHLRQQSYDVTDELPLPPGWEMTFTATGQRYFLNHIEKIT
TWQDPRKAMNQPLNHMNLHPAVSSTPVPQRSMAVSQPNLVMNHQHQQQMA
PSTLSQQNHPTQNPPAGLMSMPNALTTQQQQQQKLRLQRIQMERERIRMR
QEELMRQEAALCRQLPMEAETLAPVQAAVNPPTMTPDMRSITNNSSDPFL
NGGPYHSREQSTDSGLGLGCYSVPTTPEDFLSNVDEMDTGENAGQTPMNI
NPQQTRFPDFLDCLPGTNVDLGTLESEDLIPLFNDVESALNKSEPFLTWL

In one embodiment, the anti-WWTR/Taz antibody or antibody reagent binds to an amino acid sequence that comprises the sequence of SEQ ID NO: 4; or binds to an amino acid sequence that comprises a sequence with at least 80%, at least 85%, at least 90%, at least 95%, at least 96%, at least 97%, at least 98%, at least 99% or greater sequence identity to the sequence of SEQ ID NO: 4. In one embodiment, the anti-WWTR1/Taz antibody or antibody reagent binds to an amino acid sequence that comprises the entire sequence of SEQ ID NO: 4. In another embodiment, the antibody or antibody reagent binds to an amino acid sequence that comprises a fragment of the sequence of SEQ ID NO: 4, wherein the fragment is sufficient to bind its target, e.g., WWTR1/Taz and increase T cell activation in a population of T cells, as seen by an expression of, e.g., CD44, CD69, CD71, CD25, and/or HLA-DR.

Anti-WWTR/Taz antibodies are commercially available from, e.g., those antibodies listed in Table 2.

TABLE 2
Anti- WWTR1/Taz antibodies

| | Clone ID for | | |
|---|---|---|---|
| | monoclonal | Catalog | Reference |
| Target | antibodies | Numbers | Information |
| WWTR1/TAZ | D3I6D | 70148 | Cell signaling |
| | | | Technologies |
| WWTR1/TAZ | E5P2N | 71192 | Cell signaling |
| | | | Technologies |
| WWTR1/TAZ | V386 | 4883 | Cell signaling |
| | | | Technologies |
| WWTR1/TAZ | | ab84927 | Abcam |
| WWTR1/TAZ | | ab110239 | Abcam |
| WWTR1/TAZ | | ab224239 | Abcam |
| WWTR1/TAZ | CL0371 | ab242313 | Abcam |
| WWTR1/TAZ | | ab119373 | Abcam |
| WWTR1/TAZ | | NB110-58359 | Novus Biologicals |
| WWTR1/TAZ | | NBP1-85067 | Novus Biologicals |
| WWTR1/TAZ | CL0371 | NBP2-52926 | Novus Biologicals |
| WWTR1/TAZ | | NB600-220 | Novus Biologicals |
| WWTR1/TAZ | OTI1H9 | NBP2-01209 | Novus Biologicals |
| WWTR1/TAZ | 672027 | MAB7210 | Novus Biologicals |
| WWTR1/TAZ | | AF7210 | Novus Biologicals |
| WWTR1/TAZ | CL0370 | NBP2-30656 | Novus Biologicals |
| WWTR1/TAZ | 1F1 | SAB1402558 | Millipore Sigma |
| WWTR1/TAZ | CL0371 | AMAB90730 | Millipore Sigma |
| WWTR1/TAZ | CL0370 | AMAB90729 | Millipore Sigma |
| WWTR1/TAZ | | SAB4300644 | Millipore Sigma |
| WWTR1/TAZ | | HPA039557 | Millipore Sigma |
| WWTR1/TAZ | | T4077 | Millipore Sigma |
| WWTR1/TAZ | M2-616 | 560235 | BD Biosciences, Franklin |
| | | | lakes, New Jersey, USA |

In one embodiment, the agent that inhibits Yap is an antisense oligonucleotide. As used herein, an “antisense oligonucleotide” refers to a synthesized nucleic acid sequence that is complementary to a DNA or mRNA sequence, such as that of a microRNA. Antisense oligonucleotides are typically designed to block expression of a DNA or RNA target by binding to the target and halting expression at the level of transcription, translation, or splicing. Antisense oligonucleotides of the present invention are complementary nucleic acid sequences designed to hybridize under cellular conditions to a gene, e.g., Yap. Thus, oligonucleotides are chosen that are sufficiently complementary to the target, i.e., that hybridize sufficiently well and with sufficient specificity in the context of the cellular environment, to give the desired effect. For example, an antisense oligonucleotide that inhibits Yap may comprise at least 5, at least 10, at least 15, at least 20, at least 25, at least 30, or more bases complementary to a portion of the coding sequence of the human Yap gene, isoform 1 (e.g., SEQ ID NO: 1).

SEQ ID NO: 1 is a nucleotide sequence that encodes Yap.

| (SEQ ID NO: 1) | |
|---|---|
| | atggatcc cgggcagcag |
| 421 | ccgccgcctc aaccggcccc ccagggccaa gggcagccgc cttcgcagcc cccgcagggg |
| 481 | cagggcccgc cgtccggacc cgggcaaccg gcacccgcgg cgacccaggc ggcgccgcag |
| 541 | gcaccccccg ccgggcatca gatcgtgcac gtccgcgggg actcggagac cgacctggag |
| 601 | gcgctcttca acgccgtcat gaaccccaag acggccaacg tgccccagac cgtgcccatg |
| 661 | aggctccgga agctgcccga ctccttcttc aagccgccgg agcccaaatc ccactcccga |
| 721 | caggccagta ctgatgcagg cactgcagga gccctgactc cacagcatgt tcgagctcat |
| 781 | tcctctccag cttctctgca gttgggagct gtttctcctg ggacactgac ccccactgga |
| 841 | gtagtctctg gcccagcagc tacacccaca gctcagcatc ttcgacagtc ttcttttgag |
| 901 | atacctgatg atgtacctct gccagcaggt tgggagatgg caaagacatc ttctggtcag |
| 961 | agatacttct taaatcacat cgatcagaca acaacatggc aggaccccag gaaggccatg |
| 1021 | ctgtcccaga tgaacgtcac agcccccacc agtccaccag tgcagcagaa tatgatgaac |
| 1081 | tcggcttcag gtcctcttcc tgatggatgg gaacaagcca tgactcagga tggagaaatt |
| 1141 | tactatataa accataagaa caagaccacc tcttggctag acccaaggct tgaccctcgt |
| 1201 | tttgccatga accagagaat cagtcagagt gctccagtga aacagccacc acccctggct |
| 1261 | ccccagagcc cacagggagg cgtcatgggt ggcagcaact ccaaccagca gcaacagatg |
| 1321 | cgactgcagc aactgcagat ggagaaggag aggctgcggc tgaaacagca agaactgctt |
| 1381 | cggcaggcaa tgcggaatat caatcccagc acagcaaatt ctccaaaatg tcaggagtta |
| 1441 | gccctgcgta gccagttacc aacactggag caggatggtg ggactcaaaa tccagtgtct |
| 1501 | tctcccggga tgtctcagga attgagaaca atgacgacca atagctcaga tcctttcctt |
| 1561 | aacagtggca cctatcactc tcgagatgag agtacagaca gtggactaag catgagcagc |
| 1621 | tacagtgtcc ctcgaacccc agatgacttc ctgaacagtg tggatgagat ggatacaggt |
| 1681 | gatactatca accaaagcac cctgccctca cagcagaacc gtttcccaga ctaccttgaa |
| 1741 | gccattcctg ggacaaatgt ggaccttgga acactggaag gagatggaat gaacatagaa |
| 1801 | ggagaggagc tgatgccaag tctgcaggaa gctttgagtt ctgacatcct taatgacatg |
| 1861 | gagtctgttt tggctgccac caagctagat aaagaaagct ttcttacatg gttatag |

In one embodiment, the agent that inhibits WWTR1/Taz is an antisense oligonucleotide. For example, an antisense oligonucleotide that inhibits WWTR/Taz may comprise at least 5, at least 10, at least 15, at least 20, at least 25, at least 30, or more bases complementary to a portion of the coding sequence of the human WWTR1/Taz gene, isoform 1 (e.g., SEQ ID NO: 3).

SEQ ID NO: 3 presents that nucleic acid sequence for WWTR1, transcript variant 2.

| (SEQ ID NO: 3) | |
|---|---|
| | atg aatccggcct cggcgccccc tccgctcccg ccgcctgggc |
| 301 | agcaagtgat ccacgtcacg caggacctag acacagacct cgaagccctc ttcaactctg |
| 361 | tcatgaatcc gaagcctagc tcgtggcgga agaagatcct gccggagtct ttctttaagg |
| 421 | agcctgattc gggctcgcac tcgcgccagt ccagcaccga ctcgtcgggc ggccacccgg |
| 481 | ggcctcgact ggctgggggt gcccagcatg tccgctcgca ctcgtcgccc gcgtccctgc |
| 541 | agctgggcac cggcgcgggt gctgcgggta gccccgcgca gcagcacgcg cacctccgcc |
| 601 | agcagtccta cgacgtgacc gacgagctgc cactgccccc gggctgggag atgaccttca |
| 661 | cggccactgg ccagaggtac ttcctcaatc acatagaaaa aatcaccaca tggcaagacc |
| 721 | ctaggaaggc gatgaatcag cctctgaatc atatgaacct ccaccctgcc gtcagttcca |
| 781 | caccagtgcc tcagaggtcc atggcagtat cccagccaaa tctcgtgatg aatcaccaac |
| 841 | accagcagca gatggccccc agtaccctga gccagcagaa ccaccccact cagaacccac |
| 901 | ccgcagggct catgagtatg cccaatgcgc tgaccactca gcagcagcag cagcagaaac |
| 961 | tgcggcttca gagaatccag atggagagag aaaggattcg aatgcgccaa gaggagctca |
| 1021 | tgaggcagga agctgccctc tgtcgacagc tccccatgga agctgagact cttgccccag |
| 1081 | ttcaggctgc tgtcaaccca cccacgatga ccccagacat gagatccatc actaataata |
| 1141 | gctcagatcc tttcctcaat ggagggccat atcattcgag ggagcagagc actgacagtg |
| 1201 | gcctggggtt agggtgctac agtgtcccca caactccgga ggacttcctc agcaatgtgg |
| 1261 | atgagatgga tacaggagaa aacgcaggac aaacacccat gaacatcaat ccccaacaga |
| 1321 | cccgtttccc tgatttcctt gactgtcttc caggaacaaa cgttgactta ggaactttgg |
| 1381 | aatctgaaga cctgatcccc ctcttcaatg atgtagagtc tgctctgaac aaaagtgagc |
| 1441 | cctttctaac ctggctgtaa |

In one embodiment, Yap or WWTR1/Taz are depleted from the cell's genome using any genome editing system including, but not limited to, zinc finger nucleases, TALENS, meganucleases, and CRISPR/Cas systems. In one embodiment, the genomic editing system used to incorporate the nucleic acid encoding one or more guide RNAs into the cell's genome is not a CRISPR/Cas system; this can prevent undesirable cell death in cells that retain a small amount of Cas enzyme/protein. It is also contemplated herein that either the Cas enzyme or the sgRNAs are each expressed under the control of a different inducible promoter, thereby allowing temporal expression of each to prevent such interference. The CRISPR/Cas system is originally an RNA-mediated bacterial immune system that provides a form of acquired immunity against viruses and plasmids; it comprises three components: a Cas (CRISPR associated protein) endonuclease (such as Streptococcus pyogenes Cas9 or Francisella novicida Cas12a), a crRNA (CRISPR RNA), and a tracrRNA (transactivating crRNA). Clustered regularly interspaced short palindromic repeats (CRISPR) are short repetitions of bacterial DNA followed by short repetitions of spacer DNA from viruses or plasmids. The Cas9 endonuclease contains two nuclease domains and is programmed by a crRNA and tracrRNA hybrid to cleave the target sequence. In some embodiments, the Cas9 endonuclease is programmed by a crRNA and tracrRNA hybrid to cleave, e.g., a YAP or WWTR1/TAZ sequence. In other embodiments, the Cas9 endonuclease is programmed by a single-guide RNA (sgRNA), which contains both a crRNA and tracrRNA sequence. In some cases, the guide RNAs (gRNAs) are selected to generate a functional gene deletion, in other cases the gRNAs are selected to recruit a catalytically inactive Cas molecule to inhibit transcription of the target loci (CRISPR interference; CRISPRi) or activate transcription of human target loci (CRISPR activation; CRISPRa).

There are two main considerations in the selection of the 20-nt guide sequence for gene targeting: 1) the target sequence should precede the protospacer adjacent motif (PAM) sequence specific for the Cas nucleus used (5′-GG PAM for S. pyogenes Cas9), and 2) guide sequences should be chosen to minimize off-target activity. Guide RNA sequences can be readily generated for a given target sequence using prediction software, for example, CRISPRdirect (available on the world wide web at http://crispr.dbels.jp/), see Natio, et al. Bioinformatics. (2015) April 1; 31(7): 1120-1123; ATUMgRNA Design Tool (available on the world wide web at www.atum.bio:ecommnerce/cas9/input); an CRISPR-ERA (available on the world wide web at http://crispr-era.stanford.eduu/indexjsp), see Liu, et al. Bioinformatics, (2015) November 15; 31(22): 3676-3678. All references cited herein are incorporated herein by reference in their entireties, Non-limiting examples of publicly available gRNA design software include; sgRNA Scorer 1.0, Quilt Universal guide RNA designer, Cas-OFFinder & Cas-Designer, CRISPR-ERA, CRISPR/Cas9 target online predictor, Off-Spotter—for designing gRNAs, CRISPR MultiTargeter, ZiFiT Targeter, CRISPRdirect, CRISPR design from crispr.mit.edu/, E-CRISP etc.

Exemplary gRNA sequence for target the YAP gene, e.g., for inhibition, include:

(SEQ ID NO: 5)
5′-CATCAGATCGTGCACGTCCG-3′
(SEQ ID NO: 6)
5′-GCTGCGAAGGCGGCTGCCCT-3′
(SEQ ID NO: 7)
5′-CAGGGCCCGCCGTCCGGACC-3′
(SEQ ID NO: 8)
5′-TGCCCCAGACCGTGCCCAT-3′

Exemplary gRNA sequence for target the WWTR1/TAZ gene, e.g., for inhibition, include:

(SEQ ID NO: 9)
5′-ATCCGAAGCCTAGCTCGTGG-3′
(SEQ ID NO: 10)
5′-TGTCTAGGTCCTGCGTGACG-3′
(SEQ ID NO: 11)
5′-CTCTGCTCCCTCGAATGATA-3′
(SEQ ID NO: 12)
5′-TCTCATGTCTGGGGTCATCG-3′

A CRISPR/Cas system can be delivered using a plasmid, vector, or a ribonucleoprotein complex. Ribonucleoprotein complexes comprising a Cas protein can further comprise a nucleic acid sequence encoding crRNA and tracrRNA. When a nucleic acid encoding one or more sgRNAs and a nucleic acid encoding an RNA-guided endonuclease each need to be administered, the use of an adenovirus associated vector (AAV) is specifically contemplated. Other vectors for simultaneously delivering nucleic acids to both components of the genome editing/fragmentation system (e.g., sgRNAs, RNA-guided endonuclease) include lentiviral vectors, such as Epstein Barr, Human immunodeficiency virus (HIV), and hepatitis B virus (HBV). Each of the components of the RNA-guided genome editing system (e.g., sgRNA and endonuclease) can be delivered in a separate vector as known in the art or as described herein. Exemplary guideRNA sequences, e.g., KOgRNA and CRISPRigRNA sequences, are presented in Tables 4 and 5, respectively.

In one embodiment, the agent inhibits, e.g., Yap or WWTR1/Taz, by RNA inhibition. Inhibitors of the expression of a given gene can be an inhibitory nucleic acid. In some embodiments of any of the aspects, the inhibitory nucleic acid is an inhibitory RNA (iRNA). The RNAi can be single stranded or double stranded.

The iRNA can be siRNA, shRNA, endogenous microRNA (miRNA), or artificial miRNA. In one embodiment, an iRNA as described herein effects inhibition of the expression and/or activity of a target, e.g. Yap. In some embodiments of any of the aspects, the agent is siRNA that inhibits Yap. In some embodiments of any of the aspects, the agent is shRNA that inhibits Yap.

One skilled in the art would be able to design siRNA, shRNA, or miRNA to target Yap, e.g., using publically available design tools. siRNA, shRNA, or miRNA is commonly made using algortihms, such as RNAi Design (available, e.g., on the world wide web at maidesigner.thermofisher.com/maiexpress/ and on the world wide web at biotools.idtdna.com/site/order/designtool/index/DSIRNA_CUSTOM); Dharmacon (Layfayette, Colo.) (available, e.g., on the world wide web at https://www.thermofisher.com/order/custom-genomic-products/tools/sirna/); or Sigma Aldrich (St. Louis, Mo.) (available, e.g., on the world wide web at https://www.sigmaaldrich.com/life-science/custom-oligos/sirna-oligos/sirna-design-service.html).

In some embodiments of any of the aspects, the iRNA can be a dsRNA. A dsRNA includes two RNA strands that are sufficiently complementary to hybridize to form a duplex structure under conditions in which the dsRNA will be used. One strand of a dsRNA (the antisense strand) includes a region of complementarity that is substantially complementary, and generally fully complementary, to a target sequence. The target sequence can be derived from the sequence of an mRNA formed during the expression of the target. The other strand (the sense strand) includes a region that is complementary to the antisense strand, such that the two strands hybridize and form a duplex structure when combined under suitable conditions.

The RNA of an iRNA can be chemically modified to enhance stability or other beneficial characteristics. The nucleic acids featured in the invention may be synthesized and/or modified by methods well established in the art, such as those described in “Current protocols in nucleic acid chemistry,” Beaucage, S. L. et al. (Edrs.), John Wiley & Sons, Inc., New York, N.Y., USA, which is hereby incorporated herein by reference. Exemplary RNAi sequences, e.g., for targeting Yap or WWTR/Taz are provided herein below.

RNAi to target human YAP:
sense:
(SEQ ID NO: 13)
UGUGGAUGAGAUGGAUACA;
antisense:
(SEQ ID NO: 14)
UGUAUCCAUCUCAUCCACA
sense:
(SEQ ID NO: 15)
GACAUCUUCUGGUCAGAGA;
antisense:
(SEQ ID NO: 16)
UCUCUGACCAGAAGAUGUC
sense:
(SEQ ID NO: 17)
GGUGAUACUAUCAACCAAA;
antisense:
(SEQ ID NO: 18)
UUUGGUUGAUAGUAUCACC
sense:
(SEQ ID NO: 19)
CAAUAUGAAUAUGGAGAUC;
antisense:
(SEQ ID NO: 20)
GAUCUCCAUAUUCAUAUUG
RNAi to target human WWTR1:
sense:
(SEQ ID NO: 21)
ACGUUGACUUAGGAACUU;
antisense:
(SEQ ID NO: 22)
AAGUUCCUAAGUCAACGU
sense:
(SEQ ID NO: 23)
AGGUACUUCCUCAAUCACA;
antisense:
(SEQ ID NO: 24)
UGUGAUUGAGGAAGUACCU
sense:
(SEQ ID NO: 25)
GGUACUUCCUCAAUCACAU;
antisense:
(SEQ ID NO: 26)
AUGUGAUUGAGGAAGUACC

In one embodiment, the agent is miRNA that inhibits, e.g., Yap or WWTR1/Taz. microRNAs are small non-coding RNAs with an average length of 22 nucleotides. These molecules act by binding to complementary sequences within mRNA molecules, usually in the 3′ untranslated (3′UTR) region, thereby promoting target mRNA degradation or inhibited mRNA translation. The interaction between microRNA and mRNAs is mediated by what is known as the “seed sequence”, a 6-8-nucleotide region of the microRNA that directs sequence-specific binding to the mRNA through imperfect Watson-Crick base pairing. More than 900 microRNAs are known to be expressed in mammals. Many of these can be grouped into families on the basis of their seed sequence, thereby identifying a “cluster” of similar microRNAs. A miRNA can be expressed in a cell, e.g., as naked DNA. A miRNA can be encoded by a nucleic acid that is expressed in the cell, e.g., as naked DNA or can be encoded by a nucleic acid that is contained within a vector.

The numerous gene silencing tools described herein may result in gene of the target gene (e.g., Yap or WWTR1/Taz), such as with an RNAi molecule (e.g. siRNA or miRNA). This entails a decrease in the mRNA level in a cell for a target by at least about 5%, about 10%, about 20%, about 30%, about 40%, about 50%, about 60%, about 70%, about 80%, about 90%, about 95%, about 99%, or more of the mRNA level found in the cell without the presence of the agent. In one preferred embodiment, the mRNA levels are decreased by at least about 70%, about 80%, about 90%, about 95%, about 99%, or more. One skilled in the art will be able to readily assess whether the siRNA, shRNA, or miRNA effective target e.g., Yap, for its downregulation, for example by transfecting the siRNA, shRNA, or miRNA into cells and detecting the levels of a gene or gene product (e.g., Yap or WWTR/Taz) found within the cell via PCR-based assay or western-blotting, respectively.

The agent may be contained in, and thus further include a vector. Many such vectors useful for transferring exogenous genes into target mammalian cells are available. The vectors may be episomal, e.g. plasmids, virus-derived vectors such cytomegalovirus, adenovirus, etc., or may be integrated into the target cell genome, through homologous recombination or random integration, e.g. retrovirus-derived vectors such as MMLV, HIV-1, ALV, etc. In some embodiments, combinations of retroviruses and an appropriate packaging cell line may also find use, where the capsid proteins will be functional for infecting the target cells. Usually, the cells and virus will be incubated for at least about 24 hours in the culture medium. The cells are then allowed to grow in the culture medium for short intervals in some applications, e.g. 24-73 hours, or for at least two weeks, and may be allowed to grow for five weeks or more, before analysis. Commonly used retroviral vectors are “defective”, that is, unable to produce viral proteins required for productive infection. Replication of the vector requires growth in the packaging cell line.

The term “vector”, as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a plasmid, phage, transposon, cosmid, artificial chromosome, virus, virion, etc.

As used herein, the term “expression vector” refers to a vector that directs expression of an RNA or polypeptide (e.g., a Yap inhibitor, or agent that inhibits WWTR1/Taz) from nucleic acid sequences contained therein linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example in human cells for expression and in a prokaryotic host for cloning and amplification. The term “expression” refers to the cellular processes involved in producing RNA and proteins and as appropriate, secreting proteins, including where applicable, but not limited to, for example, transcription, transcript processing, translation and protein folding, modification and processing. “Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g. 5′ untranslated (5′UTR) or “leader” sequences and 3′ UTR or “trailer” sequences, as well as intervening sequences (introns) between individual coding segments (exons).

Integrating vectors have their delivered RNA/DNA permanently incorporated into the host cell chromosomes. Non-integrating vectors remain episomal which means the nucleic acid contained therein is never integrated into the host cell chromosomes. Examples of integrating vectors include retroviral vectors, lentiviral vectors, hybrid adenoviral vectors, and herpes simplex viral vector.

One example of a non-integrative vector is a non-integrative viral vector. Non-integrative viral vectors eliminate the risks posed by integrative retroviruses, as they do not incorporate their genome into the host DNA. One example is the Epstein Barr oriP/Nuclear Antigen-1 (“EBNA1”) vector, which is capable of limited self-replication and known to function in mammalian cells. As containing two elements from Epstein-Barr virus, oriP and EBNA1, binding of the EBNA1 protein to the virus replicon region oriP maintains a relatively long-term episomal presence of plasmids in mammalian cells. This particular feature of the oriP/EBNA1 vector makes it ideal for generation of integration-free iPSCs. Another non-integrative viral vector is adenoviral vector and the adeno-associated viral (AAV) vector.

Another non-integrative viral vector is RNA Sendai viral vector, which can produce protein without entering the nucleus of an infected cell. The F-deficient Sendai virus vector remains in the cytoplasm of infected cells for a few passages, but is diluted out quickly and completely lost after several passages (e.g., 10 passages).

Another example of a non-integrative vector is a minicircle vector. Minicircle vectors are circularized vectors in which the plasmid backbone has been released leaving only the eukaryotic promoter and cDNA(s) that are to be expressed.

As used herein, the term “viral vector” refers to a nucleic acid vector construct that includes at least one element of viral origin and has the capacity to be packaged into a viral vector particle. The viral vector can contain a nucleic acid encoding a polypeptide as described herein in place of non-essential viral genes. The vector and/or particle may be utilized for the purpose of transferring nucleic acids into cells either in vitro or in vivo. Numerous forms of viral vectors are known in the art.

### Compositions

One aspect provided herein is a composition comprising a population of activated T cells or a differentiated T cell produced using any of the methods described herein. In one embodiment, the composition is formulated for T cell transplantation; such formulation is described herein below.

Another aspect provided herein is a pharmaceutical composition comprising any of the agents that inhibits Yap, as described herein. In one embodiment, the pharmaceutical composition comprises at least 1, 2, 3, 4, 5 or more agents that inhibits Yap. When at least two agents are present in a composition, the at least two agents can be the same agent, e.g., two RNAi molecules, or different agent, e.g., a small molecule and an RNAi molecule.

Another aspect provided herein is a pharmaceutical composition comprising any of the agents that inhibits Yap and an agent that inhibits WWTR1/Taz.

In one embodiment, the compositions further comprise a pharmaceutically acceptable carrier. As used herein, the term “pharmaceutically acceptable”, and grammatical variations thereof, as they refer to compositions, carriers, diluents and reagents, are used interchangeably and represent that the materials are capable of administration to or upon a mammal without the production of undesirable physiological effects such as nausea, dizziness, gastric upset and the like. Each carrier must also be “acceptable” in the sense of being compatible with the other ingredients of the formulation. A pharmaceutically acceptable carrier will not promote the raising of an immune response to an agent with which it is admixed, unless so desired. The preparation of a pharmacological composition that contains active ingredients dissolved or dispersed therein is well understood in the art and need not be limited based on formulation. The pharmaceutical formulation contains a compound of the invention in combination with one or more pharmaceutically acceptable ingredients. The carrier can be in the form of a solid, semi-solid or liquid diluent, cream or a capsule. Typically, such compositions are prepared as injectable either as liquid solutions or suspensions, however, solid forms suitable for solution, or suspensions, in liquid prior to use can also be prepared. The preparation can also be emulsified or presented as a liposome composition. The active ingredient can be mixed with excipients which are pharmaceutically acceptable and compatible with the active ingredient and in amounts suitable for use in the therapeutic methods described herein. Suitable excipients are, for example, water, saline, dextrose, glycerol, ethanol or the like and combinations thereof. In addition, if desired, the composition can contain minor amounts of auxiliary substances such as wetting or emulsifying agents, pH buffering agents and the like which enhance the effectiveness of the active ingredient. The therapeutic composition of the present invention can include pharmaceutically acceptable salts of the components therein. Pharmaceutically acceptable salts include the acid addition salts (formed with the free amino groups of the polypeptide) that are formed with inorganic acids such as, for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like. Physiologically tolerable carriers are well known in the art. Exemplary liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes. Liquid compositions can also contain liquid phases in addition to and to the exclusion of water. Exemplary of such additional liquid phases are glycerin, vegetable oils such as cottonseed oil, and water-oil emulsions. The amount of an active agent used in the invention that will be effective in the treatment of a particular disorder or condition will depend on the nature of the disorder or condition, and can be determined by standard clinical techniques. The phrase “pharmaceutically acceptable carrier or diluent” means a pharmaceutically acceptable material, composition or vehicle, such as a liquid or solid filler, diluent, excipient, solvent or encapsulating material, involved in carrying or transporting the subject agents from one organ, or portion of the body, to another organ, or portion of the body.

Compositions described herein can be formulated for any route of administration described herein below. Methods for formulating a composition for a desired administration are further discussed below.

### T-Cell Transplantation

Described herein is a cellular therapy in which activated or differentiated T cells are transplanted, i.e., infused, to a recipient in need thereof. One aspect herein provides a method for T cell transplantation comprising (a) providing a population of T cells, (b) contacting the T cell population with an agent that inhibits Yap, (c) ex vivo culturing for a time, and under conditions sufficient to induce activation, and (d) transplanting said population of contacted T cells into a recipient subject. In another aspect, an activated or differentiated T cell produced using any method described herein can be used in a cellular therapy (e.g., T cell transplant). It is to be understood that any method of transplanting a cell, e.g., a T cell, known in the art can be used in the methods described herein. One skilled in the art would be capable of performing such transplant.

The infused cell is able to modulate the immune system to induce a response, for example, to recognize a cancer cell and target it for cellular death, in the recipient. Unlike other conventional therapies, T cells are able to replicate in vivo, resulting in long-term persistence that can lead to sustained immune response. In various embodiments, T cells administered to the recipient subject persist in the recipient subject for at least four months, five months, six months, seven months, eight months, nine months, ten months, eleven months, twelve months, thirteen months, fourteen month, fifteen months, sixteen months, seventeen months, eighteen months, nineteen months, twenty months, twenty-one months, twenty-two months, twenty-three months, two years, three years, four years, or five years after administration of the T cell to the recipient subject.

In one embodiment, the contacted T cells administered in a cellular therapy are autologous to the recipient subject. As used herein, “autologous” refers to a hematopoietic stem cell obtained from the same subject, e.g., the recipient subject

In one embodiment, the contacted T cells administered in a cellular therapy are allogeneic to the recipient subject. As used herein, “allogenic” refers to a T cell obtained from a different subject, e.g., not the recipient subject, that is a genetic match for the recipient subject.

In one embodiment, the contacted T cells administered in a cellular therapy are xenogeneic to the recipient subject. As used herein, “xenogeneic” refers to a T cell obtained from a different subject, e.g., not the recipient subject, that is a not the same species as the recipient subject.

In one aspect of the invention, the technology described herein relates to a pharmaceutical composition comprising activated or differentiated T cells as described herein, and optionally a pharmaceutically acceptable carrier for transplant to a recipient subject. The active ingredients of the pharmaceutical composition at a minimum comprise activated or differentiated T cells as described herein. In some embodiments, the active ingredients of the pharmaceutical composition consist essentially of activated or differentiated T cells as described herein. In some embodiments, the active ingredients of the pharmaceutical composition consist of activated or differentiated T cells as described herein. Pharmaceutically acceptable carriers for cell-based therapeutic formulations include saline and aqueous buffer solutions, Ringer's solution, and serum component, such as serum albumin, HDL and LDL. The terms such as “excipient”, “carrier”, “pharmaceutically acceptable carrier” or the like are used interchangeably herein. It is contemplated herein that the pharmaceutically acceptable carrier be sufficient to promote T cell health.

Suitable vehicles that can be used to provide parenteral dosage forms of activated or differentiated T cells as disclosed herein are well known to those skilled in the art. Examples include, without limitation: saline solution; glucose solution; aqueous vehicles including but not limited to, sodium chloride injection, Ringer's injection, dextrose Injection, dextrose and sodium chloride injection, and lactated Ringer's injection.

A pharmaceutical composition comprising the T cells described herein can generally be administered at a dosage of 10⁴ to 10⁹ cells/kg, 10⁵ to 10⁹ cells/kg, 10⁶ to 10⁹ cells/kg, 10⁷ to 10⁹ cells/kg, 10⁸ to 10⁹ cells/kg, 10⁵ to 10⁸ cells/kg, 10⁶ to 10⁸ cells/kg, 10⁷ to 10⁸ cells/kg, 10⁵ to 10⁷ cells/kg, 10⁶ to 10⁷ cells/kg, or 10⁵ to 10⁶ cells/kg, 10⁴ to 10⁸ cells/kg, 10⁴ to 10⁷ cells/kg, 10⁴ to 10⁶ cells/kg, 10⁴ to 10⁵ cells/kg body weight, including all integer values within those ranges. If necessary, T cell compositions can also be administered multiple times at these dosages. The cells can be administered by using infusion techniques that are commonly known in immunotherapy (see, e.g., Rosenberg et al., New Eng. J. of Med. 319:1676, 1988).

In certain aspects, it may be desired to administer activated T cells to a subject and then subsequently redraw blood (or have an apheresis performed), activate T cells therefrom as described herein, and reinfuse the patient with these activated and expanded T cells. This process can be carried out multiple times every few weeks. In certain aspects, T cells can be activated from blood draws of from 10 cc to 400 cc. In certain aspects, T cells are activated from blood draws of 20 cc, 30 cc, 40 cc, 50 cc, 60 cc, 70 cc, 80 cc, 90 cc, or 100 cc.

Modes of administration can include, for example intravenous (i.v.) injection or infusion. The compositions described herein can be administered to a patient transarterially, intratumorally, intranodally, or intramedullary. In some embodiments, the compositions of T cells may be injected directly into a tumor, lymph node, or site of infection. In one embodiment, the T cell compositions are administered into a body cavity or body fluid (e.g., ascites, pleural fluid, peritoneal fluid, or cerebrospinal fluid).

The dosage of the above treatments to be administered to a patient will vary with the precise nature of the condition being treated and the recipient of the treatment. The scaling of dosages for human administration can be performed according to art-accepted practices.

In some embodiments, a single treatment regimen is required. In others, administration of one or more subsequent doses or treatment regimens can be performed. For example, after treatment biweekly for three months, treatment can be repeated once per month, for six months or a year or longer. In some embodiments, no additional treatments are administered following the initial treatment.

### Methods of Treatment

Provided herein are methods of treating or preventing a disease or disorder caused by or associated with T cell dysfunction, the method comprising administering to a subject in need thereof a therapeutically effective amount of an activated T cell or differentiated T cell produced using any of the methods described herein, any composition described herein, or any agent that inhibits YAP described herein. In one embodiment, the methods for treating a disease or disorder further comprise administering an agent that inhibits WWTR1/Taz.

Also provided herein is a method of treating or preventing a disease or disorder caused by or associated with T cell dysfunction, the method comprising administering to a subject in need thereof a therapeutically effective amount of any agent that inhibits YAP described herein and an agent that inhibits WWTR1/Taz.

In one embodiment, the methods further comprise administering at least one second therapeutic, for example, an anti-microbial therapeutic, an anti-autoimmune disease therapeutic, and an anti-cancer therapeutic

In various aspects provided herein, co-administration of a therapeutic described herein (e.g., activated or differentiated T cell produced using any of the methods described herein, any composition described herein, or any agent that inhibits YAP described herein), and a second therapeutic enhances the efficacy of the second therapeutic. The administration of the T cells, compositions, or agents described herein modulates the immune system such that the second therapeutic has a greater effect on its target, e.g., an antibiotic has a greater effect on its target bacterium, as compared to administering the second therapeutic alone. For example, administration of the T cells, compositions, or agents described herein promotes the differentiation of T cells into memory T cells that propagate the effect of the second therapeutic, for example, an anti-cancer immunotherapy.

Provided herein is a method of treating a microbial infectious disease, the method comprising administering to a subject in need thereof an anti-microbial therapeutic; and an activated T cell or differentiated T cell produced using any of the methods described herein, any composition described herein, or any agent that inhibits YAP described herein.

Also provided herein is a method of treating an autoimmune disease, the method comprising administering to a subject in need thereof an anti-autoimmune therapeutic; and an activated T cell or differentiated T cell produced using any of the methods described herein, any composition described herein, or any agent that inhibits YAP described herein.

Further provided herein is a method of treating a cancer, the method comprising administering to a subject in need thereof an anti-cancer therapeutic; and an activated T cell or differentiated T cell produced using any of the methods described herein, any composition described herein, or any agent that inhibits YAP described herein. In various embodiments, the cancer treated herein is a carcinoma, a melanoma, a sarcoma, a myeloma, a leukemia, a lymphoma, or a solid tumor. In one embodiment, the cancer is a melanoma. In one embodiment, the cancer is a lung cancer.

A carcinoma is a cancer that originates in an epithelial tissue. Carcinomas account for approximately 80-90% of all cancers. Carcinomas can affect organs or glands capable of secretion (e.g., breasts, lung, prostate, colon, or bladder). There are two subtypes of carcinomas: adenocarcinoma, which develops in an organ or gland, and squamous cell carcinoma, which originates in the squamous epithelium. Adenocarcinomas generally occur in mucus membranes, and are observed as a thickened plaque-like white mucosa. They often spread easily through the soft tissue where they occur. Exemplary adenocarcinomas include, but are not limited to, lung cancer, prostate cancer, pancreatic cancer, esophageal cancer, and colorectal cancer. Squamous cell carcinomas can originate from any region of the body. Examples of carcinomas include, but are not limited to, prostate cancer, colorectal cancer, microsatellite stable colon cancer, microsatellite instable colon cancer, hepatocellular carcinoma, breast cancer, lung cancer, small cell lung cancer, non-small cell lung cancer, lung adenocarcinoma, melanoma, basal cell carcinoma, squamous cell carcinoma, renal cell carcinoma, ductal carcinoma in situ, ductal carcinoma.

Sarcomas are cancers that originate in supportive and connective tissues, for example bones, tendons, cartilage, muscle, and fat. Sarcoma tumors usually resemble the tissue in which they grow. Non-limiting examples of sarcomas include, Osteosarcoma or osteogenic sarcoma (originating from bone), Chondrosarcoma (originating from cartilage), Leiomyosarcoma (originating from smooth muscle), Rhabdomyosarcoma (originating from skeletal muscle), Mesothelial sarcoma or mesothelioma (originate from membranous lining of body cavities), Fibrosarcoma (originating from fibrous tissue), Angiosarcoma or hemangioendothelioma (originating from blood vessels), Liposarcoma (originating from adipose tissue), Glioma or astrocytoma (originating from neurogenic connective tissue found in the brain), Myxosarcoma (originating from primitive embryonic connective tissue), or Mesenchymous or mixed mesodermal tumor (originating from mixed connective tissue types).

Melanoma is a type of cancer forming from pigment-containing melanocytes. Melanoma typically develops in the skin, but can occur in the mouth, intestine, or eye.

Myelomas are cancers that originate in plasma cells of bone marrow. Non-limiting examples of myelomas include multiple myeloma, plasmacytoma and amyloidosis.

Lymphomas develop in the glands or nodes of the lymphatic system (e.g., the spleen, tonsils, and thymus), which purifies bodily fluids and produces white blood cells, or lymphocytes. Unlike leukemia, lymphomas form solid tumors. Lymphoma can also occur in specific organs, for example the stomach, breast, or brain; this is referred to as extranodal lymphomas). Lymphomas are subclassified into two categories: Hodgkin lymphoma and Non-Hodgkin lymphoma. The presence of Reed-Sternberg cells in Hodgkin lymphoma diagnostically distinguishes Hodgkin lymphoma from Non-Hodgkin lymphoma.

Non-limiting examples of lymphoma include Diffuse large B-cell lymphoma (DLBCL), Follicular lymphoma, Chronic lymphocytic leukemia (CLL), Small lymphocytic lymphoma (SLL), Mantle cell lymphoma (MCL), Marginal zone lymphomas, Burkitt lymphoma, hairy cell leukemia (HCL). In one embodiment, the cancer is DLBCL or Follicular lymphoma.

Leukemias (also known as “blood cancers”) are cancers of the bone marrow, which is the site of blood cell production. Leukemia is often associated with the overproduction of immature white blood cells. Immature white blood cells do not function properly, rendering the patient prone to infection. Leukemia additionally affects red blood cells, and can cause poor blood clotting and fatigue due to anemia.

In one embodiment of any aspect, the leukemia is acute myeloid leukemia (AML), Chronic myeloid leukemia (CML), Acute lymphocytic leukemia (ALL), and Chronic lymphocytic leukemia (CLL). Examples of leukemia include, but are not limited to, Myelogenous or granulocytic leukemia (malignancy of the myeloid and granulocytic white blood cell series), Lymphatic, lymphocytic, or lymphoblastic leukemia (malignancy of the lymphoid and lymphocytic blood cell series), and Polycythemia vera or erythremia (malignancy of various blood cell products, but with red cells predominating).

In one embodiment, the cancer is a solid tumor. Non-limiting examples of solid tumors include Adrenocortical Tumor, Alveolar Soft Part Sarcoma, Chondrosarcoma, Colorectal Carcinoma, Desmoid Tumors, Desmoplastic Small Round Cell Tumor, Endocrine Tumors, Endodermal Sinus Tumor, Epithelioid Hemangioendothelioma, Ewing Sarcoma, Germ Cell Tumors (Solid Tumor), Giant Cell Tumor of Bone and Soft Tissue, Hepatoblastoma, Hepatocellular Carcinoma, Melanoma, Nephroma, Neuroblastoma, Non-Rhabdomyosarcoma Soft Tissue Sarcoma (NRSTS), Osteosarcoma, Paraspinal Sarcoma, Renal Cell Carcinoma, Retinoblastoma, Rhabdomyosarcoma, Synovial Sarcoma, and Wilms Tumor. Solid tumors can be found in bones, muscles, or organs, and can be sarcomas or carinomas.

In one embodiment of any aspect, the cancer is resistant to a cancer therapy. A cancer resistant to a therapy, for example, a chemotherapy, is one that previously responded to the treatment but is now capable of growing or persisting despite the presence of continued treatment. Resistance to a therapy can occur due to, e.g., acquired mutations in the cancer cell, gene amplification in the cancer cell, or the cancer cell develops mechanisms to prevent the uptake of the treatment.

In one embodiment, the cancer is metastatic (e.g., the cancer has disseminated from its primary location to at least one secondary location).

In one embodiment, the cancer has relapsed following administration of a cancer therapy. A “relapsed cancer” is defined as the return of a disease or the signs and symptoms of a disease after a period of improvement.

In one embodiment, T cells, compositions, and agents described herein are not used to treat cancer. In one embodiment, T cells, compositions, and agents described herein are not administered to a subject for use in treating cancer. In one embodiment, T cells, compositions, and agents described herein are administered to a subject that has not been diagnosed as having, or is not at risk of having cancer.

In accordance with the invention, T cells, compositions, and agents described herein can be administered to a subject to treat or prevent a microbial infectious disease. In one embodiment, the microbial infectious disease is systemic or local.

Transmission of a microbe causing an infectious disease can occur in various manners. Transmission can occur via airborne particles or droplets. Airborne particles resulting from, e.g., coughing, sneezing, breathing, are small (<5 μm) dry and wet particles that persist in the air for long periods of time, allowing airborne contamination after the departure of the host individual. Airborne particles are <5 μm. Airborne droplets resulting from, e.g., coughing, sneezing, is caused by small (>5 μm) and often wet particles that persist in the air for a short period of time. Contamination usually occurs in the presence of the host individual. Transmission can occur via direct physical contact, e.g., touching an infected individual, including sexual contact; or indirect physical contact, e.g., by touching a contaminated surface, including soil (fomite). Fecal-oral transmission, wherein microbes in fecal particles pass from one person to the mouth of another person, is a highly common route of transmission in pediatric medicine, veterinary medicine and developing countries. Transmission can also be indirect, via another organism, either a vector (e.g. a mosquito or fly) or an intermediate host (e.g. tapeworm in pigs can be transmitted to humans who ingest improperly cooked pork). Indirect transmission could involve zoonoses or, more typically, larger pathogens like macroparasites with more complex life cycles. Transmissions can be autochthonous (i.e. between two individuals in the same place) or may involve travel of the microorganism or the affected hosts.

Exemplary microbial infectious diseases include, but are not limited to, Acute Flaccid Myelitis(AFM); Anaplasmosis; Anthrax; Babesiosis; Botulism; Brucellosis; Campylobacteriosis; Carbapenem-resistant Infection; Chancroid; Chikungunya Virus Infection (Chikungunya); Chlamydia; Ciguatera (Harmful Algae Blooms (HABs)); Clostridium Difficile Infection; Clostridium Perfringens (Epsilon Toxin); Coccidioidomycosis fungal infection (Valley fever); COVID-19 (Coronavirus Disease 2019); Creutzfeldt-Jacob Disease, transmissible spongiform encephalopathy (CJD); Cryptosporidiosis (Crypto); Cyclosporiasis; Dengue 1,2,3,4 (Dengue Fever); Diphtheria; E. coli infection, Shiga toxin-producing (STEC); Eastern Equine Encephalitis (EEE); Ebola Hemorrhagic Fever (Ebola); Ehrlichiosis; Encephalitis, Arboviral or parainfectious; Enterovirus Infection, Non-Polio (Non-Polio Enterovirus); Enterovirus Infection, D68 (EV-D68); Flea-borne spotted fever; Flinders Island spotted fever; Flying squirrel typhus; Giardiasis (Giardia); Glanders; Gonococcal Infection (Gonorrhea); Granuloma inguinale; Haemophilus Influenza disease, Type B (Hib or H-flu); Hantavirus Pulmonary Syndrome (HPS); Hemolytic Uremic Syndrome (HUS); Hepatitis A (Hep A); Hepatitis B (Hep B); Hepatitis C (Hep C); Hepatitis D (Hep D); Hepatitis E (Hep E); Herpes; Herpes Zoster, zoster VZV (Shingles); Histoplasmosis infection (Histoplasmosis); Human Immunodeficiency Virus/AIDS (HIV/AIDS); Human Papillomavirus (HPV); Influenza (Flu); Lead Poisoning; Legionellosis (Legionnaires Disease); Leprosy (Hansens Disease); Leptospirosis; Listeriosis (Listeria); Lyme Disease; Lymphogranuloma venereum infection; Malaria; Measles; Melioidosis; Meningitis, Viral (Meningitis, viral); Meningococcal Disease, Bacterial (Meningitis, bacterial); Middle East Respiratory Syndrome Coronavirus (MERS-CoV); Mumps; Norovirus; Paralytic Shellfish Poisoning (Paralytic Shellfish Poisoning, Ciguatera); Pediculosis (Lice, Head and Body Lice); Pelvic Inflammatory Disease (PID); Pertussis (Whooping Cough); Plague; Bubonic, Septicemic, Pneumonic (Plague); Pneumococcal Disease (Pneumonia); Poliomyelitis (Polio); Powassan; Psittacosis (Parrot Fever); Pthiriasis (Crabs; Pubic Lice Infestation); Pustular Rash diseases (Small pox, monkeypox, cowpox); Q-Fever; Rabies; Ricin Poisoning; Rickettsiosis (Rocky Mountain Spotted Fever); Rubella, Including congenital (German Measles); Salmonellosis gastroenteritis (Salmonella); Scabies Infestation (Scabies); Scombroid; Septic Shock (Sepsis); Severe Acute Respiratory Syndrome (SARS); Scarlet fever; Scrub typhus (Tsutsugamushi fever); Shigellosis Shigellosis gastroenteritis (Shigella); Smallpox; Staphyloccal Infection, Methicillin-resistant (MRSA); Staphylococcal Food Poisoning, Enterotoxin—B Poisoning (Staph Food Poisoning); Staphylococcal Infection, Vancomycin Intermediate (VISA); Staphylococcal Infection, Vancomycin Resistant (VRSA); Streptococcal Disease, Group A (invasive) (Strep A (invasive)); Streptococcal Disease, Group B (Strep-B); Streptococcal Toxic-Shock Syndrome, STSS, Toxic Shock (STSS, TSS); Syphilis, primary, secondary, early latent, late latent, congenital; Tetanus Infection, tetani (Lock Jaw); Trichomoniasis (Trichomonas infection); Trichonosis Infection (Trichinosis); Trench fever (five-day fever, quintan fever, urban trench fever); Tuberculosis (TB); Tuberculosis (Latent) (LTBI); Tularemia (Rabbit fever); Typhoid Fever, Group D; Typhus; Vaginosis, bacterial (Yeast Infection); Vaping-Associated Lung Injury (e-Cigarette Associated Lung Injury); Varicella (Chickenpox); Vibrio cholera (Cholera); Vibriosis (Vibrio); Viral Hemorrhagic Fever (Ebola, Lassa, Marburg); West Nile Virus; Yellow Fever; Yersenia (Yersinia); and Zika Virus Infection (Zika).

In accordance with the invention, T cells, compositions, and agents described herein can be administered to a subject to treat or prevent an autoimmune disease. Non-limiting examples of additional autoimmune disease or disorder include type 1 diabetes, inflammatory arthritis, mellitus, multiples sclerosis, psoriasis, inflammatory bowel diseases, SLE, vasculitis, allergic inflammation, such as allergic asthma, atopic dermatitis, and contact hypersensitivity, rheumatoid arthritis, multiple sclerosis (MS), lupus, systemic lupus erythematosus, Cutaneous lupus erythematosus Graves' disease (overactive thyroid), Hashimoto's thyroiditis (underactive thyroid), chronic graft v. host disease, hemophilia with antibodies to coagulation factors, celiac disease, Crohn's disease and ulcerative colitis, Guillain-Barre syndrome, primary biliary sclerosis/cirrhosis, sclerosing cholangitis, autoimmune hepatitis, Raynaud's phenomenon, scleroderma, Sjogren's syndrome, Goodpasture's syndrome, Wegener's granulomatosis, polymyalgia rheumatica, temporal arteritis/giant cell arteritis, chronic fatigue syndrome CFS), autoimmune Addison's Disease, ankylosing spondylitis, Acute disseminated encephalomyelitis, antiphospholipid antibody syndrome, aplastic anemia, idiopathic thrombocytopenic purpura, Myasthenia gravis, opsoclonus myoclonus syndrome, optic neuritis, Chronic inflammatory demyelinating polyneuropathy, Ord's thyroiditis, pemphigus, pernicious anaemia, polyarthritis in dogs, Reiter's syndrome, Takayasu's arteritis, warm autoimmune hemolytic anemia, and fibromyalgia (FM).

In one embodiment, the disease or disorder is caused by or associated with an increased Yap expression in T cells. In one embodiment, Yap expression in T cells is increased by at least 5% as compared to an appropriate control. In another embodiment, Yap expression in T cells is increased by at least by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99% or more, or at least 2×, 3×, 4×, 5×, 6×, 7×, 8×, 9×, 10×, 50×, 100×, 150×, 200×, 250×, 300×, 350×, 500×, 750× or more as compared to reference level. As used herein, a reference level refers to the level of Yap in T cells in a healthy subject, i.e., not having a disease or disorder is caused by or associated with an increased Yap expression in T cells. One skilled in the art will be capable of determining the level of Yap in a T cell e.g., by measuring the mRNA or protein level of Yap using standard techniques, e.g., PCR-based assays or western blotting, respectively.

### Increasing Vaccine Response

One aspect provided herein is a method of increasing the efficacy of a vaccine, the method comprising administering to a subject in need thereof a vaccine; and an activated T cell or differentiated T cell produced using any of the methods described herein, any composition described herein, or any agent that inhibits YAP described herein.

Vaccine efficacy can be determined by one skilled in the art by, e.g., measuring quantitative titers to show antibodies induced by the vaccine are present in the subject's blood stream. A quantitative titer should assess whether IgG antibodies that are present in the blood stream. The presence of a sufficient amount antibodies in a subject indicates that the subject is immune to the corresponding disease. As used herein, “sufficient titer” refers to the level of IgG antibodies required to immunity to given disease or pathogen. One skilled in the art can determine the sufficient titer needed for a given disease or pathogen. Vaccination or a booster dose of a vaccine is recommended if a subject does not have a sufficient titer.

In one embodiment, administering a vaccine in combination with the T cell, compositions, or agents described herein results in an increased titer (i.e., increased level of IgG antibodies) in the subject, as compared to administering the vaccine alone. In one embodiment, the titer is increased by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99% or more, or at least 2×, 3×, 4×, 5×, 6×, 7×, 8×, 9×, 10×, 50×, 100×, 150×, 200×, 250×, 300×, 350×, 500×, 750× or more when the vaccine is administered with the T cell, compositions, or agents described herein, as compared to administering the vaccine alone.

In one embodiment, administering a vaccine in combination with the T cell, compositions, or agents described herein prolongs that duration in which a sufficient titer is present in the subject, as compared to administering the vaccine alone. In one embodiment, the duration in which a sufficient titer is present is increased by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99% or more, or at least 2×, 3×, 4×, 5×, 6×, 7×, 8×, 9×, 10×, 50×, 100×, 150×, 200×, 250×, 300×, 350×, 500×, 750× or more when the vaccine is administered with the T cell, compositions, or agents described herein, as compared to administering the vaccine alone.

In one embodiment, administering a vaccine in combination with the T cell, compositions, or agents described herein decreases the time from vaccination to the presence of a sufficient titer in the subject, as compared to administering the vaccine alone. That is, the combination administration promotes more rapid production of IgG antibodies in the subject. In one embodiment, the time from vaccination to the presence of a sufficient titer is decreased by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99% or more, or at least 2×, 3×, 4×, 5×, 6×, 7×, 8×, 9×, Ox, 50×, 100×, 150×, 200×, 250×, 300×, 350×, 500×, 750× or more when the vaccine is administered with the T cell, compositions, or agents described herein, as compared to administering the vaccine alone.

Vaccines are characterized as either live attenuated vaccines or inactivated vaccines. “Live attenuated vaccines” refers to vaccine that contains whole bacteria or viruses which have been “weakened” so that they create a protective immune response but do not cause disease in healthy people. Live vaccines produce a strong and lasting immune response, however, are not suitable for subjects having a weakened or compromises immune system. Exemplary live attenuated vaccines include, but are not limited to Rotavirus vaccine, MMR vaccine, Nasal flu vaccine, Shingles vaccine, Chickenpox vaccine, BCG vaccine, Yellow fever vaccine, and Oral typhoid vaccine.

“Inactivated vaccines” refers to vaccine that contains whole bacteria or viruses which have been killed, or small parts of bacteria or viruses, such as proteins or sugars, which cannot cause disease. There are several types of inactivated vaccine, including whole killed vaccines, subunit vaccines (sometimes called ‘acellular’), toxoid vaccines, conjugate vaccines, and recombinant vaccines. Because inactivated vaccines do not contain any live bacteria or viruses, they cannot cause the diseases against which they protect, and thus are safe for subjects having a weakened or compromises immune system. However, inactivated vaccines often require repeated doses and/or booster doses. Adjuvants such as aluminium salts are often added to inactivated vaccines to help to strengthen and lengthen the immune response to the vaccine.

In one embodiment, an additional dose or booster dose of an inactivated vaccine is not required when the initial dose is administered with a T cell, composition, or agent of the invention.

Whole killed vaccines contain whole killed viruses. Exemplary whole killed vaccines include, but are not limited to, whooping cough vaccine, inactivated polio vaccine, some inactivated flu vaccines which are described as “split virion,” rabies vaccine, and Japanese encephalitis vaccine, and Hepatitis A vaccine.

Subunit vaccines, also referred to as acellular, do not contain any whole bacteria or viruses at all, but rather contain polysaccharides (sugars) or proteins from the surface of bacteria or viruses. Our immune system recognizes the polysaccharides or proteins as “foreign,” and thus can trigger an immune response which can protect against the disease.

Toxoid vaccines contain an inactivated versions of a bacterial toxins. They are called “toxoids” because they look like toxins but are not poisonous. Toxoid vaccines trigger a strong immune response. Exemplary toxoid vaccines include, but are not limited to, Diphtheria vaccine, Tetanus vaccine, Pertussis (whooping cough) vaccine.

Conjugate vaccines comprise a polysaccharide was attached (conjugated) to something known to create a strong immune response, e.g., to diphtheria or tetanus toxoid protein. The immune system recognizes these proteins very easily and this helps to generate a stronger immune response to the polysaccharide. Exemplary conjugate vaccines include, but are not limited to, Hib vaccine (a polysaccharide joined to tetanus toxoid), MenC vaccine (a polysaccharide joined to tetanus toxoid), PCV (polysaccharides from the surface of 13 types of the bacteria which causes pneumococcal disease joined to diphtheria toxoid (CRM197)), and MenACWY (polysaccharides from the surface of four types of the bacteria which causes meningococcal disease joined to diphtheria or tetanus toxoid)

Recombinant vaccines include fragments of DNA is taken from the virus or bacterium against which we want to protect. The fragments of DNA are amplified in a cell, e.g., a yeast cell, to produce large quantities of active ingredient for the vaccine (usually just a single protein or sugar). The fragment is purified and used as the active ingredient in the vaccine. Exemplary recombinant vaccines include, but are not limited to Hepatitis B vaccine, HPV vaccine, and MenB vaccine. This contains proteins from the surface of meningococcal bacteria. Three of the proteins are made using recombinant technology.

Other exemplary vaccines include inactivated flu vaccines which are described as ‘surface antigen’ (containing purified proteins from the surface of flu viruses), PPV (adult pneumococcal vaccine), which contains polysaccharides from the surface of 23 types of the bacteria which causes pneumococcal disease, and Injected typhoid vaccine (a polysaccharide vaccine).

### Administration

In some embodiments, the methods described herein relate to treating a subject having or diagnosed as having a disease or disorder caused by or associated with T cell dysfunction, e.g., cancer, an autoimmune disease, or a microbial infection, comprising administering a T cell produced using any of the methods described herein, any of the compositions described herein, or any agent, e.g., an agent that inhibits Yap, described herein. Subjects having cancer, for example, can be identified by a physician using current methods (i.e. assays) of diagnosing a condition. Symptoms and/or complications of cancer, which characterize these disease and aid in diagnosis are well known in the art and include but are not limited to, persistent cough, blood in stool, weight loss, anemia, or lumps in soft tissue. Tests that may aid in a diagnosis of, e.g. cancer, include but are not limited blood test (such as a complete blood count (CBC)), genetic testing for known cancer biomarker, biopsy of suspect tissues, and non-invasive imaging, (such a magnetic resonance imaging (MRI) or a computed tomography (CT) scan). A family history of, e.g., cancer, will also aid in determining if a subject is likely to have the disease or in making a diagnosis of cancer.

The T cells, compositions, or agents described herein can be administered to a subject having or diagnosed as having a disease or disorder caused by or associated with T cell dysfunction, e.g., cancer, an autoimmune disease, or a microbial infection. In some embodiments, the methods described herein comprise administering an effective amount of T cells, compositions, or agents to a subject in order to alleviate at least one symptom of, e.g., cancer. As used herein, “alleviating at least one symptom of cancer” is ameliorating any condition or symptom associated with, e.g., cancer (e.g., persistent cough, blood in stool, weight loss, anemia, or lumps in soft tissue). As compared with an equivalent untreated control, such reduction is by at least 5%, 10%, 20%, 40%, 50%, 60%, 80%, 90%, 95%, 99% or more as measured by any standard technique. A variety of means for administering the T cells, compositions, or agents described herein to subjects are known to those of skill in the art. In one embodiment, the T cells, compositions, or agents are administered systemically or locally (e.g., to the site of a tumor). In one embodiment, the T cells, compositions, or agents are administered intravenously. In one embodiment, the T cells, compositions, or agents are administered continuously, in intervals, or sporadically. The route of administration of the agent will be optimized for the type of therapeutic being delivered (e.g., T cells, compositions, or agents), and can be determined by a skilled practitioner.

The term “effective amount” as used herein refers to the amount of T cells, compositions, or agents that can be administered to a subject having or diagnosed as having e.g., a disease or disorder caused by or associated with T cell dysfunction, needed to alleviate at least one or more symptom associated with, e.g., cancer, an autoimmune disease, or a microbial infection. The term “therapeutically effective amount” therefore refers to an amount of T cells, compositions, or agents that is sufficient to provide, e.g., a particular anti-cancer effect when administered to a typical subject. An effective amount as used herein, in various contexts, would also include an amount of an agent sufficient to delay the development of a symptom of, e.g., cancer, alter the course of a symptom of, e.g., cancer (e.g., slowing the progression of tumor formation or metastasis), or reverse a symptom of, e.g., (e.g., shrinking or eliminating tumors). Thus, it is not generally practicable to specify an exact “effective amount”. However, for any given case, an appropriate “effective amount” can be determined by one of ordinary skill in the art using only routine experimentation.

In one embodiment, the composition or agent is administered continuously (e.g., at constant levels over a period of time). Continuous administration of an agent can be achieved, e.g., by epidermal patches, continuous release formulations, or on-body injectors.

Effective amounts, toxicity, and therapeutic efficacy can be evaluated by standard pharmaceutical procedures in cell cultures or experimental animals. The dosage can vary depending upon the dosage form employed and the route of administration utilized. The dose ratio between toxic and therapeutic effects is the therapeutic index and can be expressed as the ratio LD50/ED50. Compositions and methods that exhibit large therapeutic indices are preferred. A therapeutically effective dose can be estimated initially from cell culture assays. Also, a dose can be formulated in animal models to achieve a circulating plasma concentration range that includes the IC50 (i.e., the concentration of the agent, which achieves a half-maximal inhibition of symptoms) as determined in cell culture, or in an appropriate animal model. Levels in plasma can be measured, for example, by high performance liquid chromatography. The effects of any particular dosage can be monitored by a suitable bioassay, e.g., measuring neurological function, or blood work, among others. The dosage can be determined by a physician and adjusted, as necessary, to suit observed effects of the treatment.

### Dosage

“Unit dosage form” as the term is used herein refers to a dosage for suitable one administration. By way of example a unit dosage form can be an amount of therapeutic disposed in a delivery device, e.g., a syringe or intravenous drip bag. In one embodiment, a unit dosage form is administered in a single administration. In another, embodiment more than one unit dosage form can be administered simultaneously.

The dosage of the T cells, compositions, or agents described herein can be determined by a physician and adjusted, as necessary, to suit observed effects of the treatment. With respect to duration and frequency of treatment, it is typical for skilled clinicians to monitor subjects in order to determine when the treatment is providing therapeutic benefit, and to determine whether to administer further cells, discontinue treatment, resume treatment, or make other alterations to the treatment regimen. The dosage should not be so large as to cause adverse side effects, such as cytokine release syndrome. Generally, the dosage will vary with the age, condition, and sex of the patient and can be determined by one of skill in the art. The dosage can also be adjusted by the individual physician in the event of any complication.

Typically, the dosage ranges for an agent, e.g., a small molecule, are between 0.001 mg/kg body weight to 5 g/kg body weight, inclusive. In some embodiments, the dosage range is from 0.001 mg/kg body weight to 1 g/kg body weight, from 0.001 mg/kg body weight to 0.5 g/kg body weight, from 0.001 mg/kg body weight to 0.1 g/kg body weight, from 0.001 mg/kg body weight to 50 mg/kg body weight, from 0.001 mg/kg body weight to 25 mg/kg body weight, from 0.001 mg/kg body weight to 10 mg/kg body weight, from 0.001 mg/kg body weight to 5 mg/kg body weight, from 0.001 mg/kg body weight to 1 mg/kg body weight, from 0.001 mg/kg body weight to 0.1 mg/kg body weight, from 0.001 mg/kg body weight to 0.005 mg/kg body weight. Alternatively, in some embodiments the dosage range is from 0.1 g/kg body weight to 5 g/kg body weight, from 0.5 g/kg body weight to 5 g/kg body weight, from 1 g/kg body weight to 5 g/kg body weight, from 1.5 g/kg body weight to 5 g/kg body weight, from 2 g/kg body weight to 5 g/kg body weight, from 2.5 g/kg body weight to 5 g/kg body weight, from 3 g/kg body weight to 5 g/kg body weight, from 3.5 g/kg body weight to 5 g/kg body weight, from 4 g/kg body weight to 5 g/kg body weight, from 4.5 g/kg body weight to 5 g/kg body weight, from 4.8 g/kg body weight to 5 g/kg body weight. In one embodiment, the dose range is from 5 g/kg body weight to 30 g/kg body weight. Alternatively, the dose range will be titrated to maintain serum levels between 5 g/mL and 30 g/mL.

### Combinational Therapy

In one embodiment, the T cells, compositions, or agents described herein is used as a monotherapy, that is, no other therapeutic is administered with the T cells, compositions, or agents described herein. In one embodiment, the T cells, compositions, or agent described herein can be used in combination with other known agents and therapies for a disease or disorder caused by or associated with T cell dysfunction, e.g., an anti-cancer, an anti-autoimmune disease, or an anti-microbial therapeutic. Administered “in combination,” as used herein, means that two (or more) different treatments are delivered to the subject during the course of the subject's affliction with the disorder, e.g., the two or more treatments are delivered after the subject has been diagnosed with the disorder or disease (for example, cancer) and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery.” In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered. The T cells, compositions, or agents described herein and the at least one additional therapy can be administered simultaneously, in the same or in separate compositions, or sequentially. For sequential administration, the T cells, compositions, or agents described herein can be administered first, and the additional therapeutic can be administered second, or the order of administration can be reversed. The T cells, compositions, or agents and/or other therapeutic agents, procedures or modalities can be administered during periods of active disorder, or during a period of remission or less active disease. The agent can be administered before another treatment, concurrently with the treatment, post-treatment, or during remission of the disorder.

When administered in combination, the T cells, compositions, or agents and the at least one additional therapeutic (e.g., second or third agent), or all, can be administered in an amount or dose that is higher, lower or the same as the amount or dosage of each agent used individually, e.g., as a monotherapy. In certain embodiments, the administered amount or dosage of the agent, the additional agent (e.g., second or third agent), or all, is lower (e.g., at least 20%, at least 30%, at least 40%, or at least 50%) than the amount or dosage of each agent used individually. In other embodiments, the amount or dosage of agent, the additional agent (e.g., second or third agent), or all, that results in a desired effect (e.g., treatment of cancer, an autoimmune disease or a microbial infectious disease) is lower (e.g., at least 20%, at least 30%, at least 40%, or at least 50% lower) than the amount or dosage of each agent individually required to achieve the same therapeutic effect.

A t cell, composition, or agent described herein can be administered with an anti-cancer therapeutic. An anti-cancer therapy can be, e.g., chemotherapy, radiation therapy, chemo-radiation therapy, immunotherapy, hormone therapy, surgery or stem cell therapy. Exemplary chemotherapeutic agents include, but are not limited to, a platinum chemotherapeutic agent, an anthracyclin therapeutic agent, or an alkylating chemotherapeutic agent. Non-limiting examples of chemotherapeutic agents include an anthracycline (e.g., doxorubicin (e.g., liposomal doxorubicin)), a vinca alkaloid (e.g., vinblastine, vincristine, vindesine, vinorelbine), an alkylating agent (e.g., cyclophosphamide, decarbazine, melphalan, ifosfamide, temozolomide), an immune cell antibody (e.g., alemtuzamab, gemtuzumab, rituximab, tositumomab), an antimetabolite (including, e.g., folic acid antagonists, pyrimidine analogs, purine analogs and adenosine deaminase inhibitors (e.g., fludarabine)), an mTOR inhibitor, a TNFR glucocorticoid induced TNFR related protein (GITR) agonist, a proteasome inhibitor (e.g., aclacinomycin A, gliotoxin or bortezomib), an immunomodulator such as thalidomide or a thalidomide derivative (e.g., lenalidomide). General chemotherapeutic agents considered for use in combination therapies include anastrozole (Arimidex®), bicalutamide (Casodex®), bleomycin sulfate (Blenoxane®), busulfan (Myleran®), busulfan injection (Busulfex®), capecitabine (Xeloda®), N4-pentoxycarbonyl-5-deoxy-5-fluorocytidine, carboplatin (Paraplatin®), carmustine (BiCNU®), chlorambucil (Leukeran®), cisplatin (Platinol®), cladribine (Leustatin®), cyclophosphamide (Cytoxan® or Neosar®), cytarabine, cytosine arabinoside (Cytosar-U®), cytarabine liposome injection (DepoCyt®), dacarbazine (DTIC-Dome®), dactinomycin (Actinomycin D, Cosmegan), daunorubicin hydrochloride (Cerubidine®), daunorubicin citrate liposome injection (DaunoXome®), dexamethasone, docetaxel (Taxotere®), doxorubicin hydrochloride (Adriamycin®, Rubex®), etoposide (Vepesid®), fludarabine phosphate (Fludara®), 5-fluorouracil (Adrucil®, Efudex®), flutamide (Eulexin®), tezacitibine, Gemcitabine (difluorodeoxycitidine), hydroxyurea (Hydrea®), Idarubicin (Idamycin®), ifosfamide (IFEX®), irinotecan (Camptosar®), L-asparaginase (ELSPAR®), leucovorin calcium, melphalan (Alkeran®), 6-mercaptopurine (Purinethol®), methotrexate (Folex®), mitoxantrone (Novantrone®), mylotarg, paclitaxel (Taxol®), phoenix (Yttrium90/MX-DTPA), pentostatin, polifeprosan 20 with carmustine implant (Gliadel®), tamoxifen citrate (Nolvadex®), teniposide (Vumon®), 6-thioguanine, thiotepa, tirapazamine (Tirazone®), topotecan hydrochloride for injection (Hycamptin®), vinblastine (Velban®), vincristine (Oncovin®), and vinorelbine (Navelbine®). Exemplary alkylating agents include, without limitation, nitrogen mustards, ethylenimine derivatives, alkyl sulfonates, nitrosoureas and triazenes): uracil mustard (Aminouracil Mustard®, Chlorethaminacil®, Demethyldopan®, Desmethyldopan®, Haemanthamine®, Nordopan®, Uracil nitrogen Mustard®, Uracillost®, Uracilmostaza®, Uramustin®, Uramustine®), chlormethine (Mustargen®), cyclophosphamide (Cytoxan®, Neosar®, Clafen®, Endoxan®, Procytox®, Revimmune™), ifosfamide (Mitoxana®), melphalan (Alkeran®), Chlorambucil (Leukeran®), pipobroman (Amedel®, Vercyte®), triethylenemelamine (Hemel®, Hexalen®, Hexastat®), triethylenethiophosphoramine, Temozolomide (Temodar®), thiotepa (Thioplex®), busulfan (Busilvex®, Myleran®), carmustine (BiCNU®), lomustine (CeeNU®), streptozocin (Zanosar®), and Dacarbazine (DTIC-Dome®). Additional exemplary alkylating agents include, without limitation, Oxaliplatin (Eloxatin®); Temozolomide (Temodar® and Temodal®); Dactinomycin (also known as actinomycin-D, Cosmegen®); Melphalan (also known as L-PAM, L-sarcolysin, and phenylalanine mustard, Alkeran®); Altretamine (also known as hexamethylmelamine (HMM), Hexalen®); Carmustine (BiCNU®); Bendamustine (Treanda®); Busulfan (Busulfex® and Myleran®); Carboplatin (Paraplatin®); Lomustine (also known as CCNU, CeeNU®); Cisplatin (also known as CDDP, Platinol® and Platinol®-AQ); Chlorambucil (Leukeran®); Cyclophosphamide (Cytoxan® and Neosar®); Dacarbazine (also known as DTIC, DIC and imidazole carboxamide, DTIC-Dome®); Altretamine (also known as hexamethylmelamine (HMM), Hexalen®); Ifosfamide (Ifex®); Prednumustine; Procarbazine (Matulane®); Mechlorethamine (also known as nitrogen mustard, mustine and mechloroethamine hydrochloride, Mustargen®); Streptozocin (Zanosar®); Thiotepa (also known as thiophosphoamide, TESPA and TSPA, Thioplex®); Cyclophosphamide (Endoxan®, Cytoxan®, Neosar®, Procytox®, Revimmune®); and Bendamustine HCl (Treanda®). Exemplary mTOR inhibitors include, e.g., temsirolimus; ridaforolimus (formally known as deferolimus, (1R,2R,45)-4-[(2R)-2 [(1R,95,125,15R,16E,18R,19R,21R,235,24E,26E,28Z,305,325,35R)-1,18-dihydroxy-19,30-dimethoxy-15,17,21,23, 29,35-hexamethyl-2,3,10,14,20-pentaoxo-11,36-dioxa-4-azatricyclo[30.3.1.04′9]hexatriaconta-16,24,26,28-tetraen-12-yl]propyl]-2-methoxycyclohexyl dimethylphosphinate, also known as AP23573 and MK8669, and described in PCT Publication No. WO 03/064383); everolimus (Afinitor® or RADOOl); rapamycin (AY22989, Sirolimus®); simapimod (CAS 164301-51-3); emsirolimus, (5-{2,4-Bis[(35)-3-methylmorpholin-4-yl]pyrido[2,3-(i]pyrimidin-7-yl}-2-methoxyphenyl)methanol (AZD8055); 2-Amino-8-[iraw5,-4-(2-hydroxyethoxy)cyclohexyl]-6-(6-methoxy-3-pyridinyl)-4-methyl-pyrido[2,3-JJpyrimidin-7(8H)-one (PF04691502, CAS 1013101-36-4); and N2-[1,4-dioxo-4-[[4-(4-oxo-8-phenyl-4H-1-benzopyran-2-yl)morpholinium-4-yl]methoxy]butyl]-L-arginylglycyl-L-α-aspartylL-serine-, inner salt (SF1126, CAS 936487-67-1), and XL765. Exemplary immunomodulators include, e.g., afutuzumab (available from Roche®); pegfilgrastim (Neulasta®); lenalidomide (CC-5013, Revlimid®); thalidomide (Thalomid®), actimid (CC4047); and IRX-2 (mixture of human cytokines including interleukin 1, interleukin 2, and interferon γ, CAS 951209-71-5, available from IRX Therapeutics). Exemplary anthracyclines include, e.g., doxorubicin (Adriamycin® and Rubex®); bleomycin (Lenoxane®); daunorubicin (dauorubicin hydrochloride, daunomycin, and rubidomycin hydrochloride, Cerubidine®); daunorubicin liposomal (daunorubicin citrate liposome, DaunoXome®); mitoxantrone (DHAD, Novantrone®); epirubicin (Ellence™); idarubicin (Idamycin®, Idamycin PFS®); mitomycin C (Mutamycin®); geldanamycin; herbimycin; ravidomycin; and desacetylravidomycin. Exemplary vinca alkaloids include, e.g., vinorelbine tartrate (Navelbine®), Vincristine (Oncovin®), and Vindesine (Eldisine®)); vinblastine (also known as vinblastine sulfate, vincaleukoblastine and VLB, Alkaban-AQ® and Velban®); and vinorelbine (Navelbine®). Exemplary proteosome inhibitors include bortezomib (Velcade®); carfilzomib (PX-171-007, (5)-4-Methyl-N-((5)-1-(((5)-4-methyl-1-((R)-2-methyloxiran-2-yl)-1-oxopentan-2-yl)amino)-1-oxo-3-phenylpropan-2-yl)-2-((5)-2-(2-morpholinoacetamido)-4-phenylbutanamido)-pentanamide); marizomib (NPT0052); ixazomib citrate (MLN-9708); delanzomib (CEP-18770); and O-Methyl-N-[(2-methyl-5-thiazolyl)carbonyl]-L-seryl-O-methyl-N-[(llS′)-2-[(2R)-2-methyl-2-oxiranyl]-2-oxo-1-(phenylmethyl)ethyl]-L-serinamide (ONX-0912).

One of skill in the art can readily identify a chemotherapeutic agent of use with methods and compositions describe herein (e.g. see Physicians' Cancer Chemotherapy Drug Manual 2014, Edward Chu, Vincent T. DeVita Jr., Jones & Bartlett Learning; Principles of Cancer Therapy, Chapter 85 in Harrison's Principles of Internal Medicine, 18th edition; Therapeutic Targeting of Cancer Cells: Era of Molecularly Targeted Agents and Cancer Pharmacology, Chs. 28-29 in Abeloff's Clinical Oncology, 2013 Elsevier; and Fischer D S (ed): The Cancer Chemotherapy Handbook, 4th ed. St. Louis, Mosby-Year Book, 2003).

Radiation therapy, according to the invention disclosed herein, encompasses both non-invasive (external) and invasive (internal) radiation therapies. In an external radiation therapy, treatment is affected by radiation sources outside the body, whereas in an invasive radiation therapy treatment is affected by radiation sources planted inside the body. The representative diseases treated by non-invasive or invasive radiation therapy include, for example, cancer, rheumatoid arthritis, angioplasty, or restenosis.

A chemo-radiation therapy, e.g., a combination of a chemotherapy and radiation therapy, in combination with the compositions described herein.

As used herein, “immunotherapy” refers to a treatment designed, e.g., to increase the immune system of a subject to stop or slow the growth of cancer cells, stop the metastasis of cancer cells, and/or target the cancer cell for programmed cell death. Exemplary immunotherapies include a monoclonal antibody, a non-specific immunotherapy, an oncolytic virus therapy, adoptive T-cell therapy (e.g., adoptive CD4⁺ or CD8⁺ effector T cell therapy), adopted natural killer (NK) cell therapy, adopted NK T cell therapy and cancer (e.g., tumor) vaccines.

In accordance with one embodiment, the subject is administered a non-specific immunotherapy in combination with the compositions described herein. Two common non-specific immunotherapies include, e.g., interferons and interleukins. Interferons (such as Roferon-A [2α], Intron A [2β], Alferon [2α]) boost the immune system to target cancer cells for programmed cell death, and/or slow the growth of cancer cells. Interleukins (such as interleukin-2, IL-2, or aldesleukin (Proleukin)) boost the immune system to produce cells that target cancer cells for programmed cell death. Interleukins are used to treat, e.g., kidney cancer and skin cancer, including melanoma. Non-specific immunotherapies can be administered as a monotherapy, or administered after or at the same time as another anti-cancer therapy, such as chemotherapy or radiation therapy.

In accordance with one embodiment, the subject is administered an oncolytic virus in combination with the compositions described herein. Oncolytic virus therapy utilizes a genetically modified virus (e.g., a herpes simplex virus, or other virus) to target cancer cells for programmed cell death via an immune response. An oncolytic virus is administered locally, e.g., injected into a tumor, where the virus enters the cancer cells and replicates. The replication can result in lysis of the cancer cells, resulting in the release of antigens and activating an immune response that targets the cancer cells for programmed cell death. Administration of the virus can be repeated until the desired effect is obtained (e.g., the tumor is eradicated). Oncolytic virus therapy (e.g., talimogene laherparepvec (Imlygic), or T-VEC) has been approved for treatment of melanoma.

In one embodiment, a subject having cancer is administered a cancer vaccine in combination with the compositions described herein. Cancers that can be treated with and/or prevented by cancer vaccines include but are not limited to bladder cancer, brain tumors, breast cancer, cervical cancer, colorectal cancer, kidney cancer, leukemia, lung cancer, melanoma, myeloma, pancreatic cancer, and prostate cancer.

In one embodiment, a subject having cancer is administered an adoptive NK cell therapy in combination with the compositions described herein. Natural killer (NK) cells are immune cells that function to target a cancer cell for programmed cell death without requiring prior sensitization to a tumor antigen. NK target cancer cells throughavarietyofmechanisms, e.g., through receptor-mediated cytotoxicity. NK cells express a germ-line encoded receptors, such as the c-type lectin homodimer, NKG2D, which binds to stress induced ligands (e.g., ULBP's, MICA/MICB) expressed on tumor cells. Upon ligation, NK cells degranulate, releasing perforin and granzymes to induce target cell apoptosis. NK cell degranulation can also be triggered though a process called antibody dependent cell-mediated cytotoxicity (ADCC). NK cells and T cells can be modified (e.g., with cytokines such as IL-2, IL-12, IL-15, or IL-18) to increase their cancer cell capabilities and specificity. NK cells administered to a subject can be autologous or allogeneic. NK cells administered to a subject can be expand in vivo or ex vivo. Cancers that can be treated with adoptive NK cell or T cell therapy include, but are not limited to advanced melanoma, renal cell carcinoma, acute myeloid leukemia, lymphoma, solid tumors, non-Hodgkin's lymphoma, chronic lymphocytic leukemia, non-B lineage hematologic malignancies, Her2⁺ breast cancer, and Her2⁺ gastric cancer. The use of adoptive NK cell and adoptive NK T cell therapies are further reviewed in, e.g., Davis, Z B, et al. Cancer J. 2015 November-December; 21(6): 486-491, which is incorporated by reference herein in its entirety.

In one embodiment, adoptive T cell therapy, e.g., CD4+ or CD8+ effector T cell therapy, or NK T cell therapy, is reactive with tumor antigens. T cells for adoptive T cell therapies can be are purified from, e.g, tumor tissue, blood, or other patient tissue. Purified T cells can be e.g., activated, expanded, and/or genetically modified, e.g., ex vivo in cell culture. Activated, expanded, and/or genetically modified T cells can be e.g., administered into the patient, for example, by intravenous injection or other acceptable routes, in combination with compositions described herein. It is envisioned that the agent that inhibits activity of CBM signalosome would to enhance recruitment of the administered cells to the site of the tumor.

In accordance with one embodiment, the subject is administered a hormone therapy in combination with the compositions described herein. Hormone therapy is designed to add, block, or remove hormones from the body to, e.g., halt or slow the growth of cancer cells. Hormone therapy can include administration of, e.g., progesterone, oophorectomy, tamoxifen, gonadotropin-releasing hormone (GnRH) agonists or analogues and androgen therapy. Hormone therapy can also refer to removing glands, e.g., thyroid, pancreas, and ovary, to reduce the levels of hormones in the body. Hormone therapies are known in the art and can be administered by a skilled person.

In accordance with one embodiment, the subject is administered a stem cell therapy in combination with the compositions described herein. Stem cell therapy can comprise removing a subjects stem cells prior to receiving treatment to destroy all stem cells (e.g., chemotherapy, radiotherapy, or a combination thereof). Stems cells can be re-administered to the patient following such treatment (e.g., a stem cell transplant). A stem cell transplant can be autologous, or allogenic. A stem cell transplant can be a tandem transplant (e.g., two or more transplants in a row), a mini-transplant (e.g., a subject's immune system is suppressed less than a typical transplant), or a syngeneic stem cell transplant (e.g., allogenic stem cells received from an identical twin). Cancers that can be treated with stem cell therapy include but are not limited to leukemias, lymphomas, multiple myeloma, testicular cancer, neuroblastoma, and certain childhood cancers.

A T cell, composition, or agent described herein can be administered with an anti-autoimmune therapeutic. Exemplary therapeutics used to treat autoimmune disease include, but are not limited to, corticosteroids, e.g. prednisone (Deltasone®, Orasone®) budesonide (Entocort EC®), prednisolone (Millipred®); Janus kinase inhibitors, e.g., tofacitinib (Xeljanz®); Calcineurin inhibitors, e.g., cyclosporine (Neoral® Sandimmune®, SangCyak, tacrolimus (AstagrafXLk. Envarsus XR®, Prografk); mTOR inhbitors, e.g., sirolimus (Rapamune) everoimus (Afinitor®, Zortressk); IMDH inhibitors, e.g. azathioprine (Azasan®, Inuran®), leflunonide (Arava®), nycophenolate (CellCept®, Myfortic®); biologics, e.g., abatacept (Orencia®), adalimumab (Hunira®), anakinra (Kineret®), certolizumab (CimziaR), etanercept (EnbrelR), golimumab (SimponiR), infliximab (Remicade®), ixekizumab (TaltzA), natalizumab (Tysabri®), rituximab (RituxanJ), secukinumab (Cosentyx), tocilizumab (Actemraa), ustekinunab (StelaraR vedolizunab (Entyvioa); and Monoclonal antibodies, e.g., basiliximab (Simulect®), and daclizumab (Zinbrytaa).

A T cell, composition, or agent described herein can be administered with an anti-microbial therapeutic. Exemplary therapeutics used to treat microbial infectious disease include, but are not limited to, an anti-fungal therapeutic, anti-bacterial therapeutic, such as an antibiotic or anti-bacterial agent, or an anti-viral therapeutic. Exemplary anti-fungal therapeutics include, but are not limited to clotrimazole, econazole, miconazole, terbinafine, fluconazole, ketoconazole, and amphotericin.

Exemplary antibiotics include, but are not limited to Aknilox, Ambisome, Amoxycillin, Ampicillin, Augmentin, Avelox, Azithromycin, Bactroban, Betadine, Betnovate, Blephamide, Cefaclor, Cefadroxil, Cefdinir, Cefepime, Cefix, Cefixime, Cefoxitin, Cefpodoxime, Cefprozil, Cefuroxime, Cefzil, Cephalexin, Cephazolin, Ceptaz, Chloramphenicol, Chlorhexidine, Chloromycetin, Chlorsig, Ciprofloxacin, Clarithromycin, Clindagel, Clindamycin, Clindatech, Cloxacillin, Colistin, Co-trimoxazole, Demeclocycline, Diclocil, Dicloxacillin, Doxycycline, Duricef, Erythromycin, Flamazine, Floxin, Framycetin, Fucidin, Furadantin, Fusidic, Gatifloxacin, Gemifloxacin, Gemifloxacin, llosone, Iodine, Levaquin, Levofloxacin, Lomefloxacin, Maxaquin, Mefoxin, Meronem, Minocycline, Moxifloxacin, Myambutol, Mycostatin, Neosporin, Netromycin, Nitrofurantoin, Norfloxacin, Norilet, Ofloxacin, Omnicef, Ospamox, Oxytetracycline, Paraxin, Penicillin, Pneumovax, Polyfax, Povidone, Rifadin, Rifampin, Rifaximin, Rifinah, Rimactane, Rocephin, Roxithromycin, Seromycin, Soframycin, Sparfloxacin, Staphlex, Targocid, Tetracycline, Tetradox, Tetralysal, tobramycin, Tobramycin, Trecator, Tygacil, Vancocin, Velosef, Vibramycin, Xifaxan, Zagam, Zitrotek, Zoderm, Zymar, and Zyvox.

Exemplary anti-bacterial agents include, but is not limited to aminoglycosides (e.g., amikacin (Amikina), gentamicin (Garamycina), kanamycin (Kantrexa), neomycin (Mycifradina), netilmicin (Netromycina), tobramycin (Nebcina), Paromomycin (Humatina)), ansamycins (e.g., geldanamycin, herbimycin), carbacephem (e.g., loracarbef (Lorabida), Carbapenems (e.g., ertapenem (lnvanza), doripenem (Doribaxa), imipenem/cilastatin (Primaxin), meropenem (Merrema), cephalosporins (first generation) (e.g., cefadroxil (Duricef®), cefazolin (Ancef®), cefalotin or cefalothin (Keflin®), cefalexin (Keflex®), cephalosporins (second generation) (e.g., cefaclor (Ceclor®), cefamandole (Mandol®), cefoxitin (Mefoxin®), cefprozil (Cefzil®), cefuroxime (Ceftin®, Zinnat®)), cephalosporins (third generation) (e.g., cefixime (Suprax®), cefdinir (Omnicef®, Cefdiel®), cefditoren (Spectracef®), cefoperazone (Cefobid®), cefotaxime (Ciaforan®), cefpodoxime (Vantin®), ceftazidime (Fortaz®), ceftibuten (Cedax®), ceftizoxime (Cefizox®), ceftriaxone (Rocephin®)), cephalosporins (fourth generation) (e.g., cefepime (Maxipime®)), cephalosporins (fifth generation) (e.g., ceftobiprole (Zeftera®)), glycopeptides (e.g., teicoplanin (Targocid®), vancomycin (Vancocin®), telavancin (Vibativ®)), lincosamides (e.g., clindamycin (Cieocin®), lincomycin (Lincocin®)), lipopeptide (e.g., daptomycin (Cubicin®)), macrolides (e.g., azithromycin (Zithromax®, Surnamed®, Zitrocin®), clarithromycin (Biaxin®), dirithromycin (Dynabac®), erythromycin (Erythocin®, Erythroped®), roxithromycin, troleandomycin (Tao®), telithromycin (Ketek®), spectinomycin (Trobicin®)), monobactams (e.g., aztreonam (Azactam®)), nitrofurans (e.g., furazolidone (Furoxone®), nitrofurantoin (Macrodantin, Macrobid®)), penicillins (e.g., amoxicillin (Novamox®, Amoxil®), ampicillin (Principen®), azlocillin, carbenicillin (Geocillin®), cloxacillin (Tegopen®), dicloxacillin (Dynapen®), flucloxacillin (Fioxapen®), mezlocillin (Mezlin®), methicillin (Staphcillin®), nafcillin (Unipen®), oxacillin (Prostaphlin®), penicillin G (Pentids®), penicillin V (Pen-Vee-K®), piperacillin (Pipracil®), temocillin (Negaban®), ticarcillin (Ticar®)), penicillin combinations (e.g., amoxicillin/clavulanate (Augmentin®), ampicillin/sulbactam (Unasyn®), piperacillin/tazobactam (Zosyn®), ticarcillin/clavulanate (Timentin®)), polypeptides (e.g., bacitracin, colistin (Coly-Mycin-S®), polymyxin B, quinolones (e.g., ciprofloxacin (Cipro®, Ciproxin®, Ciprobay®), enoxacin (Penetrex®), gatifloxacin (Tequin®), levofloxacin (Levaquin®), lomefloxacin (Maxaquin®), moxifloxacin (Avelox®), nalidixic acid (Neggram®), norfloxacin (Noroxin®), ofloxacin (Fioxin®, Ocuflox®), trovafloxacin (Trovan®), grepafloxacin (Raxar®), sparfloxacin (Zagam®), temafloxacin (Omniflox®)), sulfonamides (e.g., mafenide (Sulfamylon®), sulfonamidochrysoidine (Prontosil®), sulfacetamide (Sulamyd®, Bleph-10®), sulfadiazine (Micro-Sulfon®), silver sulfadiazine (Silvadene®), sulfamethizole (Thiosulfil Forte®), sulfamethoxazole (Gantanol®), sulfanilimide, sulfasalazine (Azulfidine®), sulfisoxazole (Gantrisin®), trimethoprim (Proloprim®), Trimpex®), trimethoprim-sulfamethoxazole (co-trimoxazole) (TMP-SMX) (Bactrim®, Septra®)), tetracyclines (e.g., demeclocycline (Declomycin®), doxycycline (Vibramycin®), minocycline (Minocin®), oxytetracycline (Terramycin®), tetracycline (Sumycin®, Achromycin® V, Steclin®)), drugs against mycobacteria (e.g., clofazimine (Lamprene®), dapsone (Avlosulfon®), capreomycin (Capastat®), cycloserine (Seromycin®), ethambutol (Myambutol®), ethionamide (Trecator®), isoniazid (I.N.H.®), pyrazinamide (Aidinamide®), rifampin (Rifadin®, Rimactane®), rifabutin (Mycobutin®), rifapentine (Priftin®), streptomycin), and others (e.g., arsphenamine (Salvarsan®), chloramphenicol (Chloromycetin®), fosfomycin (Monurol®), fusidic acid (Fucidin®), linezolid (Zyvox®), metronidazole (Fiagyl®), mupirocin (Bactroban®), platensimycin, quinupristin/dalfopristin (Synercid®), rifaximin (Xifaxan®), thiamphenicol, tigecycline (Tigacyl®), and tinidazole (Tindamax®, Fasigyn®).

Exemplary anti-viral therapeutics include, but are not limited to Abacavir, Acyclovir (Aciclovir), Adefovir, Amantadine, Ampligen, Amprenavir (Agenerase), Arbidol, Atazanavir, Atripla, Balavir, Baloxavir marboxil (Xofluza®), Biktarvy Boceprevir (Victrelis®), Cidofovir, Cobicistat (Tybost®), Combivir (fixed dose drug), Daclatasvir (Daklinza®), Darunavir, Delavirdine, Descovy, Didanosine, Docosanol, Dolutegravir, Doravirine (Pifeltro®), Ecoliever, Edoxudine, Efavirenz, Elvitegravir, Emtricitabine, Enfuvirtide, Entecavir, Etravirine (Intelence®), Famciclovir, Fomivirsen, Fosamprenavir, Foscamet, Fosfonet, Fusion inhibitor, Ganciclovir (Cytovene®), Ibacitabine, Ibalizumab (Trogarzo®), Idoxuridine, Imiquimod, Imunovir, Indinavir, Inosine, Integrase inhibitor, Interferon type I, Interferon type II, Interferon type III, Interferon, Lamivudine, Letermovir (Prevymis®), Lopinavir, Loviride, Maraviroc, Methisazone, Moroxydine, Nelfinavir, Nevirapine, Nexavir®, Nitazoxanide, Norvir, Nucleoside analogues, Oseltamivir (Tamiflu®), Peginterferon alfa-2a, Peginterferon alfa-2b, Penciclovir, Peramivir (Rapivab®), Pleconaril, Podophyllotoxin, Protease inhibitor (pharmacology), Pyramidine, Raltegravir, Remdesivir, Reverse transcriptase inhibitor, Ribavirin, Rilpivirine (Edurant®), Rimantadine, Ritonavir, Saquinavir, Simeprevir (Olysio®), Sofosbuvir, Stavudine, Synergistic enhancer (antiretroviral), Telaprevir, Telbivudine (Tyzeka®), Tenofovir alafenamide, Tenofovir disoproxil, Tenofovir, Tipranavir, Trifluridine, Trizivir, Tromantadine, Truvada, Valaciclovir (Valtrex), Valganciclovir, Vicriviroc, Vidarabine, Viramidine, Zalcitabine, Zanamivir (Relenza®), and Zidovudine.

### Parenteral Dosage Forms

Parenteral dosage forms of T cells, compositions, or agents described herein can be administered to a subject by various routes, including, but not limited to, subcutaneous, intravenous (including bolus injection), intramuscular, and intraarterial. Since administration of parenteral dosage forms typically bypasses the patient's natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, controlled-release parenteral dosage forms, and emulsions.

Suitable vehicles that can be used to provide parenteral dosage forms of the disclosure are well known to those skilled in the art. Examples include, without limitation: sterile water; water for injection USP; saline solution; glucose solution; aqueous vehicles such as but not limited to, sodium chloride injection, Ringer's injection, dextrose Injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate.

### Controlled and Delayed Release Dosage Forms

In some embodiments of the aspects described herein, an T cells, compositions, or agents are administered to a subject by controlled- or delayed-release means. Ideally, the use of an optimally designed controlled-release preparation in medical treatment is characterized by a minimum of drug substance being employed to cure or control the condition in a minimum amount of time. Advantages of controlled-release formulations include: 1) extended activity of the drug; 2) reduced dosage frequency; 3) increased patient compliance; 4) usage of less total drug; 5) reduction in local or systemic side effects; 6) minimization of drug accumulation; 7) reduction in blood level fluctuations; 8) improvement in efficacy of treatment; 9) reduction of potentiation or loss of drug activity; and 10) improvement in speed of control of diseases or conditions. (Kim, Cherng-ju, Controlled Release Dosage Form Design, 2 (Technomic Publishing, Lancaster, Pa.: 2000)). Controlled-release formulations can be used to control a compound of formula (I)'s onset of action, duration of action, plasma levels within the therapeutic window, and peak blood levels. In particular, controlled- or extended-release dosage forms or formulations can be used to ensure that the maximum effectiveness of an agent is achieved while minimizing potential adverse effects and safety concerns, which can occur both from under-dosing a drug (i.e., going below the minimum therapeutic levels) as well as exceeding the toxicity level for the drug.

A variety of known controlled- or extended-release dosage forms, formulations, and devices can be adapted for use with any agent described herein. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,733,566; and 6,365,185, each of which is incorporated herein by reference in their entireties. These dosage forms can be used to provide slow or controlled-release of one or more active ingredients using, for example, hydroxypropylmethyl cellulose, other polymer matrices, gels, permeable membranes, osmotic systems (such as OROS® (Alza Corporation, Mountain View, Calif. USA)), multilayer coatings, microparticles, liposomes, or microspheres or a combination thereof to provide the desired release profile in varying proportions. Additionally, ion exchange materials can be used to prepare immobilized, adsorbed salt forms of the disclosed compounds and thus effect controlled delivery of the drug. Examples of specific anion exchangers include, but are not limited to, DUOLITE® A568 and DUOLITE® AP143 (Rohm&Haas, Spring House, Pa. USA).

### Efficacy

The efficacy of T cells, compositions, or agents described herein, e.g., for the treatment of a disease or disorder caused by or associated with T cell dysfunction, e.g., an anti-cancer, an anti-autoimmune disease, or an anti-microbial therapeutic, can be determined by the skilled practitioner. However, a treatment is considered “effective treatment,” as the term is used herein, if one or more of the signs or symptoms of, e.g., cancer, are altered in a beneficial manner, other clinically accepted symptoms are improved, or even ameliorated, or a desired response is induced e.g., by at least 10% following treatment according to the methods described herein. Efficacy can be assessed, for example, by measuring a marker, indicator, symptom, and/or the incidence of a condition treated according to the methods described herein or any other measurable parameter appropriate, e.g., decreased tumor size, decreases metastatsis. Efficacy can also be measured by a failure of an individual to worsen as assessed by hospitalization, or need for medical interventions (i.e., progression of increased tumor size and presence of metastasis). Methods of measuring these indicators are known to those of skill in the art and/or are described herein.

Efficacy can be assessed in animal models of a condition described herein, for example, a mouse model or an appropriate animal model of a disease or disorder caused by or associated with T cell dysfunction, e.g., a cancer, an autoimmune disease, or a microbial infectious disease, as the case may be. When using an experimental animal model, efficacy of treatment is evidenced when a statistically significant change in a marker is observed, e.g., reduced tumor size, reduced metastasis, reduced expression of known cancer markers.

All patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

### EXAMPLES

### Example 1: YAP Affects T-Cell Responses in Cancer and Infectious Disease

The transcriptional regulators yes-associated protein (YAP) and transcriptional coactivator with PDZ-binding motif (TAZ) play essential roles in animal development and homeostasis [1, 2]. YAP and TAZ are paralogs that function as key effectors of the Hippo signaling pathway, mediating transcriptional responses downstream of a variety of cellular cues, including changes in the mechanical microenvironment, cell polarity, extracellular matrix, actin cytoskeleton and growth factors. When they localize the nucleus YAP and TAZ direct gene expression changes that impact cell proliferation, survival and fate.

The role of YAP or TAZ in regulating T cell effector functions is vague and understudied. Here, the inventors investigated the role of Yap in primary CD4+ and CD8+ T-cell responses-specifically, activation, proliferation, and differentiation into effector T-cell subsets—and how Yap affects T-cell responses in cancer, influenza and pneumonia.

Described are results of experiments that show that YAP functions as a novel immunomodulatory factor in T cells, and that inhibition of YAP activity enhances CD4⁺ and CD8⁺ T cell activation, differentiation and function in vitro and in vivo. Specifically, the inventors have found that deletion of YAP in T cells reduces tumor growth and that YAP deletion increases memory T cell generation in mouse models of influenza and pneumococcal infections.

Yap Suppresses T Cell Function and Infiltration in the Tumor Environment

The inventors observed that Yap levels are elevated upon T-cell activation and that conditional deletion of the Yap gene in CD4⁺ and CD8⁺ T cells enhanced their activation and differentiation potential. Specifically, these phenotypes translated in vivo to reduced growth of B16F10 melanoma and Lewis lung carcinoma (LLC) lung cancer tumors in mice with Yap-deleted T cells, with these mice showing notably increased T-cell infiltration into tumors. Using adoptive T-cell transfer experiments, the inventors observed that Yap-deleted polyclonal CD8⁺ T cells have an intrinsic ability to infiltrate tumors with higher efficiency. RNA sequencing (RNA-seq) analyses of Yap-deleted tumor-infiltrating lymphocytes (TILs) revealed up-regulation of key signals important for CD4⁺ and CD8⁺ T-cell activation, differentiation, and function. Notably, the inventors found that Yap-regulated gene expression changes were tumor specific, as the inventors observed minimal gene expression changes in lymphocytes isolated from tumor-draining lymph nodes (TDLNs). Yap-regulated gene expression signatures from TILs correlated with T-cell infiltration and patient survival across multiple human cancers in The Cancer Genome Atlas (TCGA), including melanoma and lung cancer, consistent with the inventor's mouse studies.

Deletion of Yap Increases Memory T Cell Generation in Infectious Mouse Models.

Further, the inventors have found that YAP inhibits CD4+ and CD8+ effector memory and tissue resident memory T cell generation during infection. Specifically, the inventors have found that deletion of YAP increases memory T cell generation in two in vivo mouse models: a viral infection with influenza (flu; strain PR8) and a bacterial infection with S. pneumonia, serotype 19 (SP19).

These discoveries have important consequences for immunotherapy and adoptive transfer of CAR-Ts, TCR-T cells and Tumor inflitrating Lymphocytes (TILs) against specific antigens in cancer, autoimmune diseases or viral infections, where YAP inhibition ex vivo leads to an increase of the success rate of these therapies by increasing T cell activation, migration, and cytotoxicity. Specifically, the inventor's results highlight Yap as a broad suppressor of CD4⁺ and CD8⁺ T-cell activation and function and a key regulator of T-cell tumor infiltration and survival in cancer immunotherapy patients. The inventor's findings show YAP as an inhibitor of T cell activation, differentiation, migration and function. Lastly, since YAP/TAZ deletion increases the number of effector memory and resident memory T cells in response to both bacterial and viral infections, YAP inhibition can also be utilized in vaccine development against infectious agents.

### Results

Yap Inhibits CD4⁺ and CD8⁺ T-Cell Activation

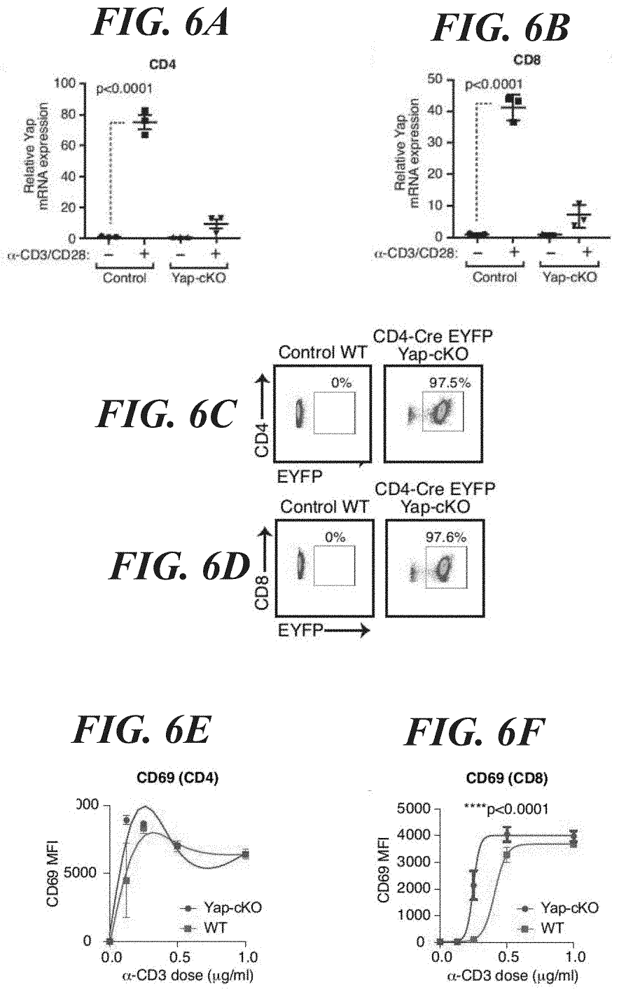

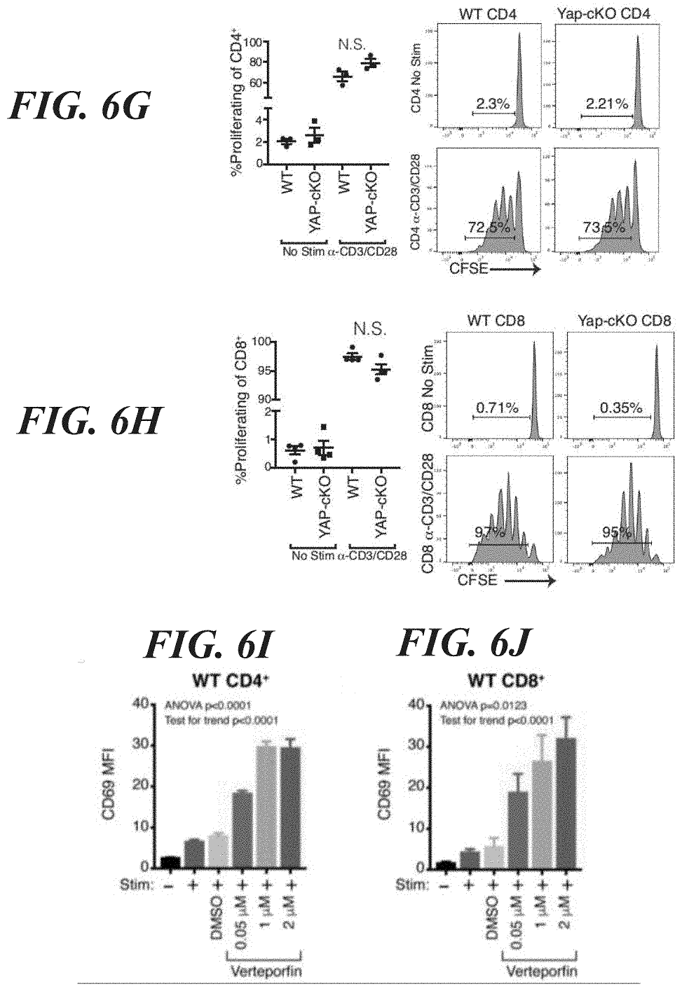

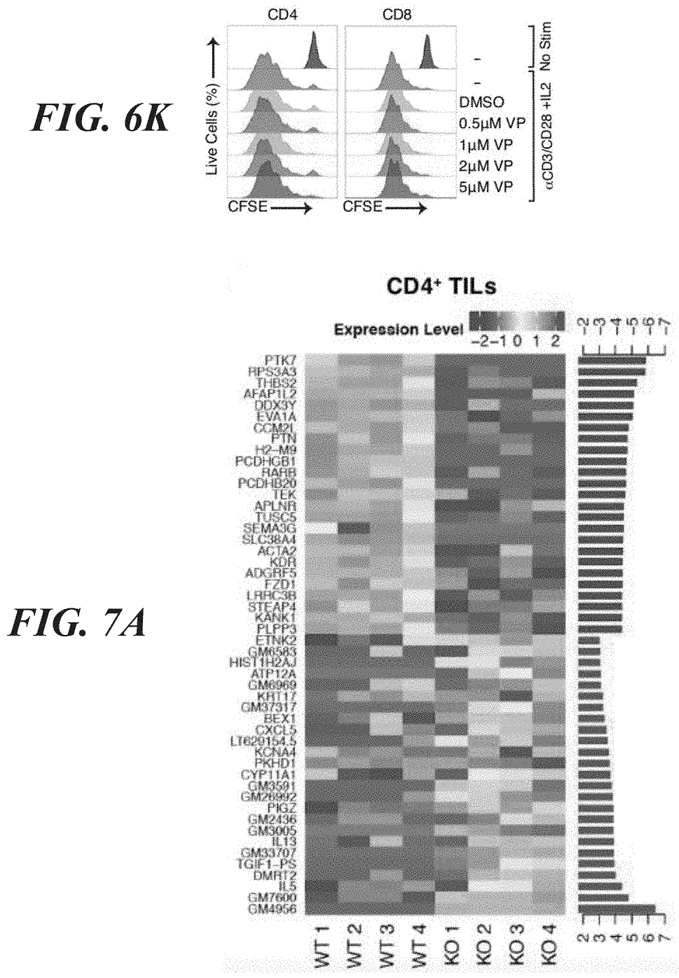

To study the role of Yap in T cells, the inventors analyzed Yap levels in isolated mouse primary CD4⁺ and CD8⁺ T cells that were either unstimulated or activated with anti-cluster of differentiation 3(CD3)/cluster of differentiation 28 (CD28) coated beads across a range of time points up to 24 hours. The inventors observed rapid induction of Yap protein in both CD4⁺ and CD8⁺ T cells upon in vitro activation (FIG. 1A and FIG. 1B). This observation contrasted with prior reports that Yap is exclusively expressed in Tregs or CD8⁺ T cells cultured under specific conditions [27,37], which prompted us to systematically explore roles for Yap in CD4⁺ and CD8⁺ T-cell activation and function. To gain insight into the roles of Yap in T cells, the inventors generated a mouse model in which Yap deletion and enhanced yellow fluorescent protein (EYFP) expression are induced under the control of the CD4 promoter (Yap-locus of X-over P1 (loxP)/loxP; lox-stop-lox (LSL)-EYFP; CD4-Cre, herein referred to as Yap-conditional knockout [cKO]). Yap expression was efficiently reduced in both CD4⁺ and CD8⁺ T cells in Yap-cKO mice, which was expected given the activity of this Cre model at the CD4⁺CD8⁺ double-positive (DP) stage of T-cell development (FIG. 6A and FIG. 6B), and cells were also efficiently marked by EYFP expression (FIG. 1C and FIG. 1D). No systemic defects or gross phenotypes were observed in Yap-cKO mice housed in a barrier facility. Due to the double positive stage of T cell development, deletion of the YAP gene was achieved in both CD4+ and CD8+ T cells, as demonstrated by EYFP expression and loss of YAP mRNA in both T cell populations (FIG. 6A-FIG. 6D). To assess whether YAP expression is controlled by T cell activation, the inventors isolated CD4+ and CD8+ T cells and activated them using αCD3 and αCD28 coated magnetic beads for 24 hours. The inventors found that YAP mRNA levels sharply increased following T cell activation (FIG. 6C and FIG. 6D) showing a role for YAP in activated T cells.

The inventors next measured the surface levels of activation markers cluster of differentiation 44 (CD44), cluster of differentiation 69 (CD69), and cluster of differentiation 25 (CD25) in Yap-cKO T cells [38-41]. CD4⁺ and CD8⁺ T cells were isolated from wild-type (WT) and Yap-cKO mouse spleens, activated by increasing concentrations of plate-bound anti-CD3, and levels of activation markers were measured 72 hours later. The inventors found that Yap-cKO CD4⁺ and CD8⁺ T cells were more sensitive to anti-CD3 stimulation compared to WT cells, showing increased expression of CD44 (FIG. 1C and FIG. 1D), CD25 (FIG. 1E and FIG. 1F), and CD69 (FIG. 6E and FIG. 6F). CD44 and CD25 in CD4⁺ and CD8⁺ T cells exhibited significantly higher expression under all stimulation conditions tested, with CD69 being significantly up-regulated only after stimulation with intermediate anti-CD3 concentrations (0.25 and 0.5 μg/mL for CD8⁺ T cells, and 0.125 μg/mL for CD4⁺ T cells). CD4⁺ and CD8⁺ T-cell proliferation was also measured, but no significant differences were observed between WT and Yap-cKO cells 72 hours post stimulation (FIG. 6G and FIG. 6H). These data show that Yap plays an inhibitory role in T-cell activation and that loss of Yap enhances the sensitivity of CD4⁺ and CD8⁺ T cells to T cell receptor (TCR) signaling.

Yap functions as a transcriptional regulator, with the best characterized roles being the regulation of the TEA domain family member (TEAD) transcriptional factors. The inventors observed a substantial increase in the expression of TEAD1 and TEAD3 in CD4⁺ T cells (FIG. 1G) and TEAD1 in CD8⁺ T cells upon activation (FIG. 1H). These increases in TEAD expression prompted us to test the effects of the small-molecule drug verteporfin (also known as Visudyne)—which has been reported to inhibit Yap-mediated activation of TEADs [42]-on T-cell activation and proliferation. Verteporfin treatment of CD4⁺ and CD8⁺ T cells isolated from WT mice increased levels of early activation markers CD71 (FIG. 1I, FIG. 1J) and CD69 (FIG. 6I, FIG. 6J, FIG. 11E and FIG. 11F) in a concentration-dependent manner. The inventors also found elevated CD25 expression in YAP-cKO CD4+ T cells and increased CD69 levels in YAP-cKO CD8+ T cells compared to the respective WT cells (FIG. 11A-11D). Similar to the inventors observations with Yap-cKO cells, verteporfin treatment did not significantly affect proliferation of T cells isolated from WT mice 3 days post stimulation (FIG. 6K). These data show that Yap-regulated transcription plays a suppressive function following T-cell activation.

Yap Inhibits CD4⁺ T-Cell Differentiation

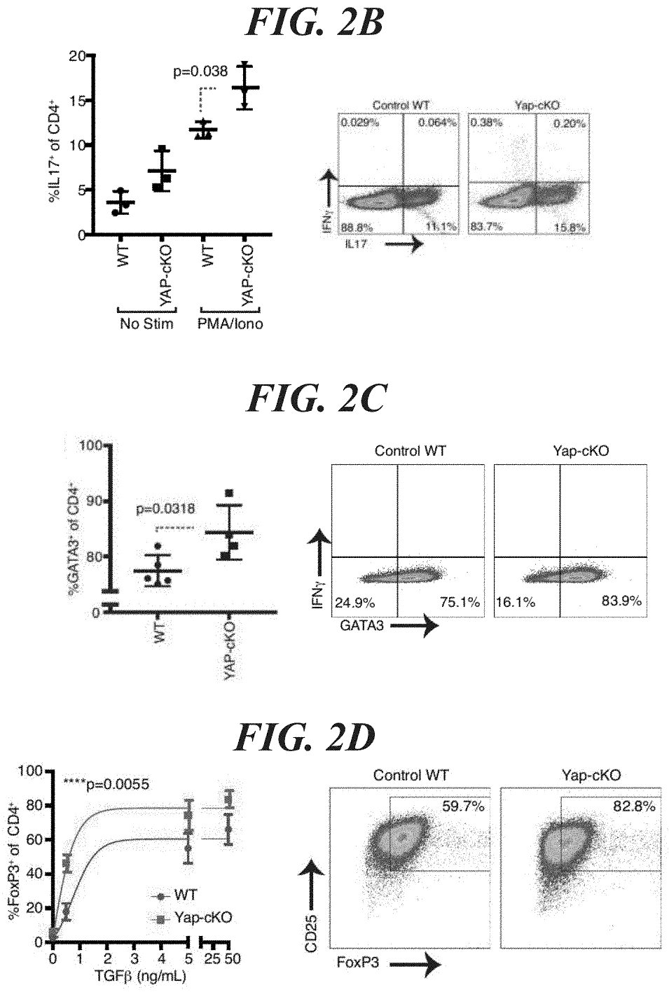

Upon encountering their cognate antigen and receiving appropriate costimulation, naïve CD4⁺ T cells become activated and can differentiate into several functionally diverse subsets, including Th1, Th2, Th17, and Tregs. These subsets contribute to protective immunity or immunopathology depending on microenvironmental signals [3]. Signature transcription factors and effector cytokines define each subset: Th1 is defined by expression of T-BET and interferon gamma (IFNγ), Th2 by GATA binding protein 3 (GATA3) and interleukin 4 (IL-4), Th17 by RORγt and IL-17, and Treg by forkhead box protein 3 (Foxp3). Given the wealth of evidence for Yap playing key roles in stem cell regulation, the inventors hypothesized that deletion of Yap alters the differentiation potential of CD4⁺ T cells. To test this hypothesis, the inventors isolated naïve CD4⁺ T cells from WT and Yap-cKO mice and cultured them under Th1-, Th17-, Th2-, and Treg-polarizing conditions [43,44]. The inventors found that Yap-deleted CD4⁺ T cells showed significantly enhanced Th1 (FIG. 2A), Th17 (FIG. 2B), Th2 (FIG. 2C), and Tregs (FIG. 2D) differentiation compared to WT cells, demonstrated by increased intracellular IFNγ, IL-17, GATA3, and Foxp3 expression, respectively. The inventors also found elevated CD25 expression in YAP-cKO CD4+ T cells and increased CD69 levels in YAP-cKO CD8+ T cells compared to the respective WT cells (FIGS. 11A-11D). The inventors observed higher Foxp3 induction at lower concentrations of TGFβ in Yap-cKO cells compared to WT controls, indicating that Yap-deleted cells were more responsive to Treg differentiation conditions. Therefore, YAP acts as an inhibitor of CD4+ T cell differentiation, with loss of YAP potentially decreasing the threshold for differentiation in response to polarizing conditions. Collectively, these observations are distinct from prior findings that showed that Yap functions only in Tregs [37] and implicate Yap as an inhibitor of CD4+ T-cell activation and differentiation into Th1, Th17, Th2, and Treg fates.

Yap Deletion does not Alter T-Cell Development or Output in the Thymus

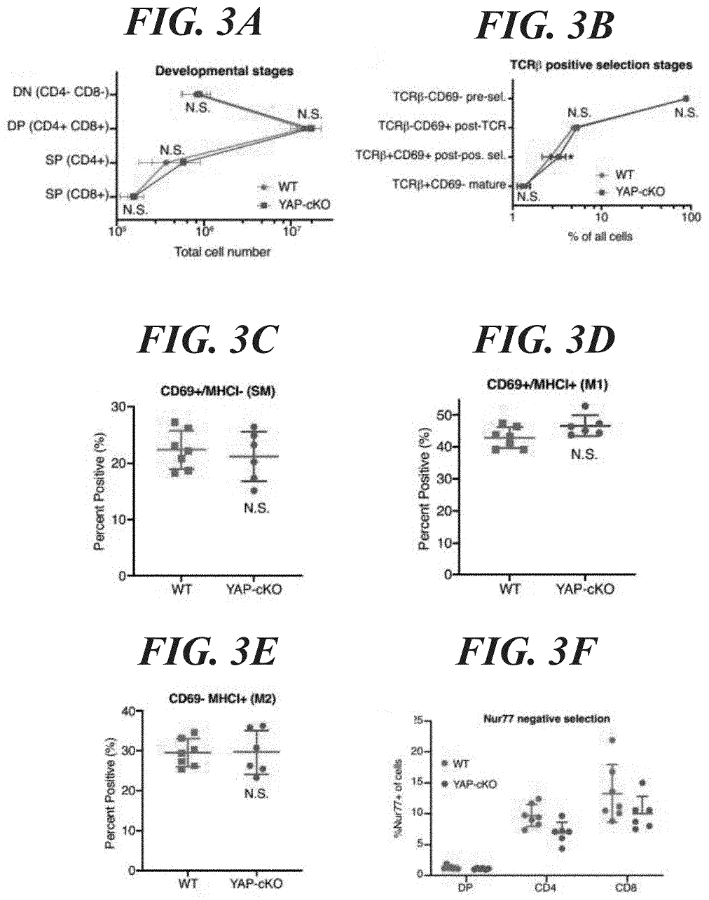

Next, the inventors compared thymic populations from WT and Yap-cKO mice to determine whether alterations in T-cell development may play a role in the observed functional changes. During maturation in the thymus, precursor CD4⁺CD8⁺ DP thymocytes go through positive selection to identify thymocytes expressing TCRs that can functionally bind peptide-major histocompatibility complex (MHC) complexes in the cortex [45]. Positively selected thymocytes become single positive (SP) and migrate from the cortex to the medulla, where self-reactive T cells die during negative selection, while surviving T cells exit into systemic circulation. Negative selection eliminates T cells with high affinity for self-peptide/self-MHC to reduce the potential for autoimmunity. Total cell counts in WT and Yap-cKO mice were determined across the continuum of thymocyte maturation, including double-negative (DN) (CD4⁻CD8⁻), DP (CD4⁺CD8⁺), and SP (CD4⁺ or CD8⁺) T cells (FIG. 3A). No significant changes were observed in total cell count at any stage, showing that output from positive and negative selection processes were not broadly changed in Yap-cKO mice compared to WT.

Next, the inventors analyzed the developmental progression of thymocytes through positive selection in greater detail using TCRβ and CD69 as markers. Yap-cKO mice compared to WT mice had no significant changes in the percentage of preselection (TCRβ⁻CD69⁻), post-TCR engagement (TCRβ⁻CD69⁺), or mature (TCRβ⁺CD69⁻) thymocytes (FIG. 3B). There were significantly more Yap-cKO thymocytes in the post-positive-selection stage (TCRβ⁺CD69⁺) compared to WT mice (approximately 1% increase in total cells). However, thymocyte frequency in the next maturation stage (TCRβ⁺CD69⁻) was similar between Yap-cKO and WT mice, indicating no significant change in output of mature TCRβ⁺CD69⁻ cells. Overall, these observations show comparable selection output and TCRβ selection between Yap-cKO and WT mice.

Positively selected SP thymocytes (TCRβ⁺CCR7⁺) can be further functionally defined using markers CD69 and MHC class I (MHCI; H-2Kb) [46]. The inventors observed no significant differences between Yap-cKO and WT mouse thymocyte frequency in CD69⁺MHCI⁻ semimature (SM) (FIG. 3C), CD69⁺MHCI⁺ mature 1 (M1) (FIG. 3D), and CD69-MHCI+ mature 2 (M2) (FIG. 3E) stages. CD69 and nuclear receptor 77 (Nur77) are both markers of recent TCR engagement and rapidly degrade after signal removal [47,48]. However, CD69 also controls thymocyte emigration and exit from the medulla, while Nur77 is more specifically involved in negative selection [49-51]. In this role, Nur77 controls T-cell survival during negative selection to eliminate any T cells with high affinity binding to self-peptide-MHC and can induce apoptosis in immature thymocytes [52,53]. The inventors therefore compared Nur77 levels in Yap-cKO with WT in DP and SP thymocytes to assess negative selection. The inventor's analysis showed no increase in Nur77 expression in Yap-cKO mice compared to WT (FIG. 3F), showing that Yap-cKO thymocytes do not receive prolonged TCR signaling relative to WT cells. Altogether, the inventors data from thymic populations show that Yap deletion does not significantly change the number or percentage of cells during thymocyte development or thymic output.

Deletion of Yap in T Cells Promotes T-Cell Infiltration into Solid Tumors and Blocks Tumor Growth

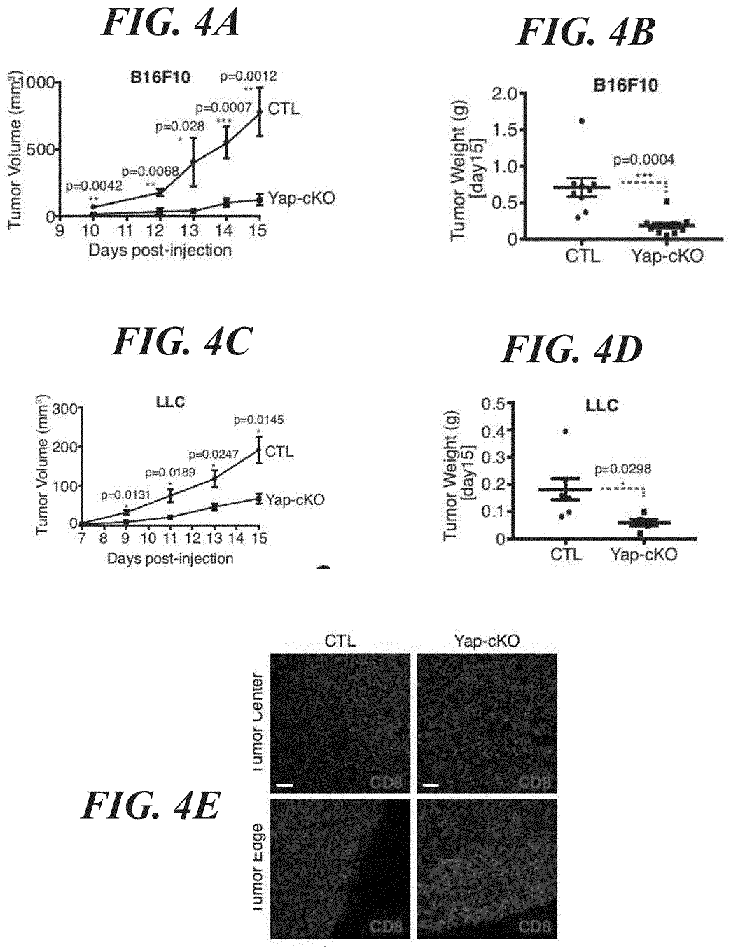

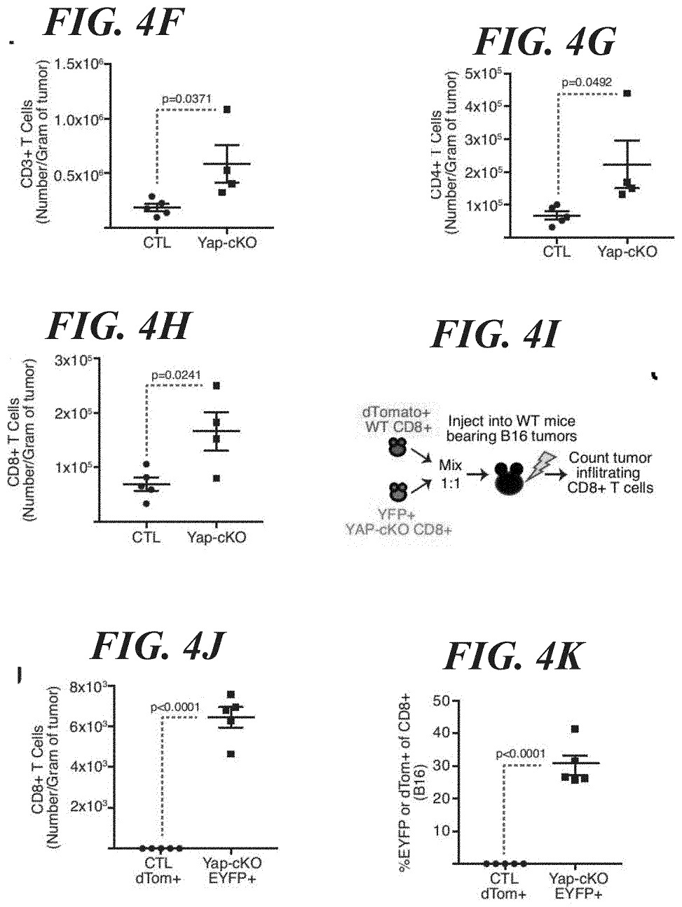

Having observed that Yap inhibits T-cell activation and differentiation in vitro, the inventors decided to test the effect of Yap deletion in T cells following an immune challenge in vivo. For this, the inventors decided to test antitumor T-cell responses using the B16F10 tumor model, which was chosen because of the associated poorly immunogenic phenotype and the highly immunosuppressive microenvironment that leads to low T-cell infiltration [54-57]. Yap deletion in T cells resulted in superior antitumor immunity, as evidenced by the significant delay in tumor growth in Yap-cKO compared to WT mice (FIG. 4A and FIG. 4B), consistent with prior observations [37]. The inventors observed similar reduced growth of subcutaneous LLC tumors in Yap-cKO mice (FIG. 4C and FIG. 4D), showing a general role for Yap in antitumor T-cell responses.

Given the strong correlation between CD8⁺ T-cell tumor infiltration and patient survival, and CD8⁺ T-cell tumor infiltration and patient responses to immunotherapy [15-19,58-62], the inventors investigated the extent of T-cell infiltration in tumors that developed in Yap-cKO versus WT mice. Immunofluorescence microscopy analysis revealed that tumors that developed in Yap-cKO mice were significantly more infiltrated with CD8⁺ T cells at both the tumor center and tumor edge compared to WT mice (FIG. 4E). Flow cytometry analysis revealed more Yap-cKO CD4⁺ and CD8⁺ T cells infiltrating tumors compared to WT counterparts (FIG. 4F and FIG. 4H).

To directly address whether Yap-deleted T cells have an increased ability to infiltrate tumors, the inventors isolated polyclonal CD8⁺ T cells from Yap-cKO and WT mice and directly compared tumor-infiltrating capacity in adoptive T-cell transfer experiments in WT mice inoculated with B16F10 tumor cells (illustrated in FIG. 4I). The inventors isolated tdTomato⁺ CD8⁺ T cells from WT mice and EYFP+CD8⁺ T cells from Yap-cKO mice and mixed them at a 1 to 1 ratio. Cell mixtures were intravenously injected into WT mice the same day as subcutaneous injection of B16F10 cells. Absolute numbers of tdTomato⁺ (WT) and EYFP⁺ (Yap-cKO) CD8⁺ T cells were then measured in tumors after 15 days. Yap-cKO CD8⁺ T cells showed a significantly enhanced capacity to infiltrate tumors (FIG. 4J), with nearly 30% of all CD8⁺ tumor-infiltrating T cells being EYFP⁺ Yap-cKO T cells compared to almost undetectable numbers of tdTomato⁺ WT T cells (FIG. 4K). These data are the first to conclusively show that Yap-cKO CD8⁺ T cells have intrinsically enhanced tumor infiltration capacity. Further, by performing in vivo adoptive cell transfer (ACT) experiments the inventors found that YAP-deleted CD8+ T cells exhibit an enhanced ability to infiltrate B16 melanoma tumors compared to wild type cells.

Yap Regulates Global T-Cell Responses in the Local Tumor Microenvironment

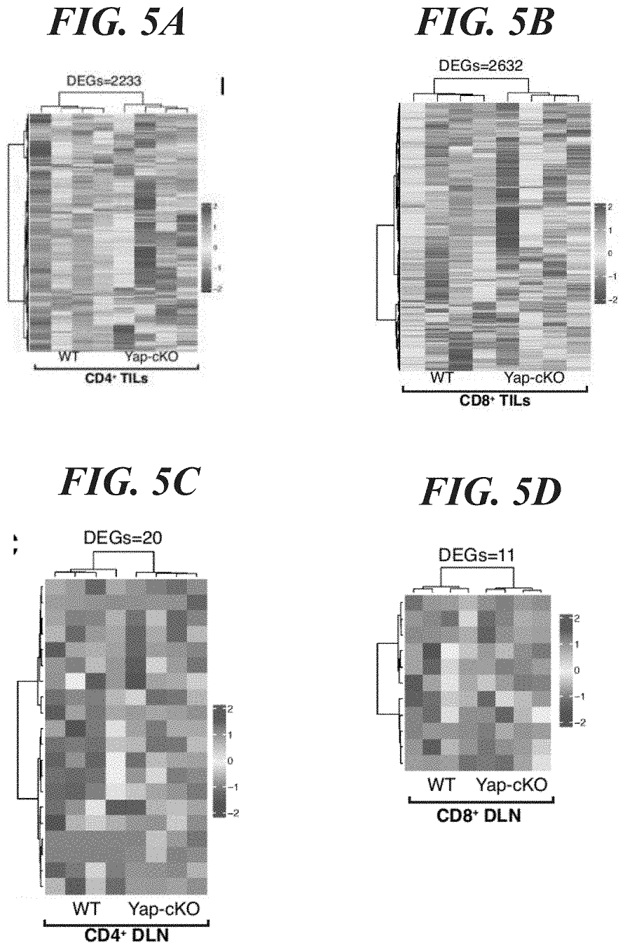

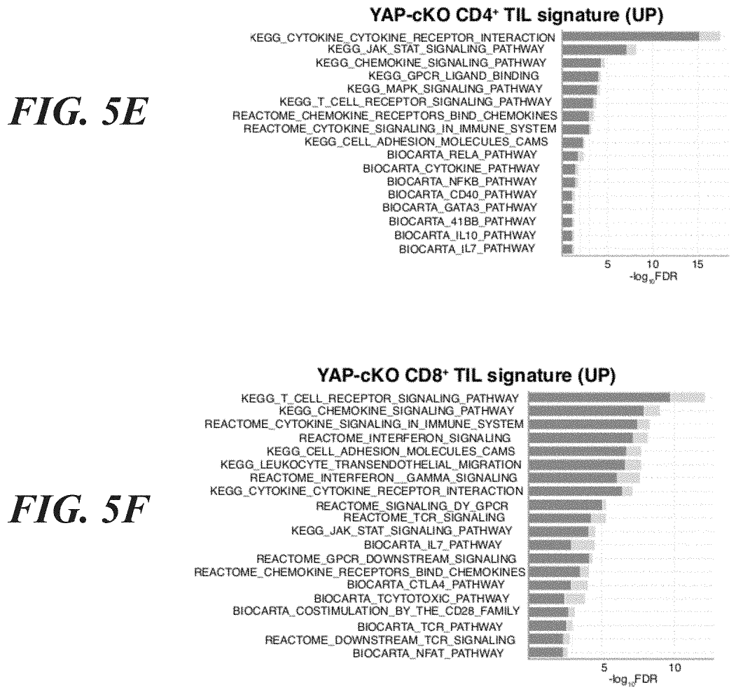

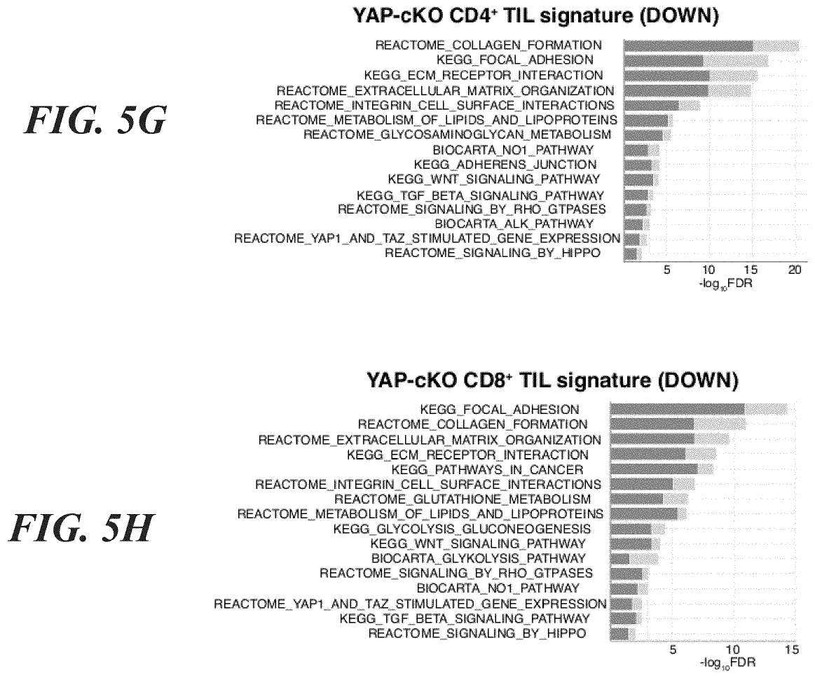

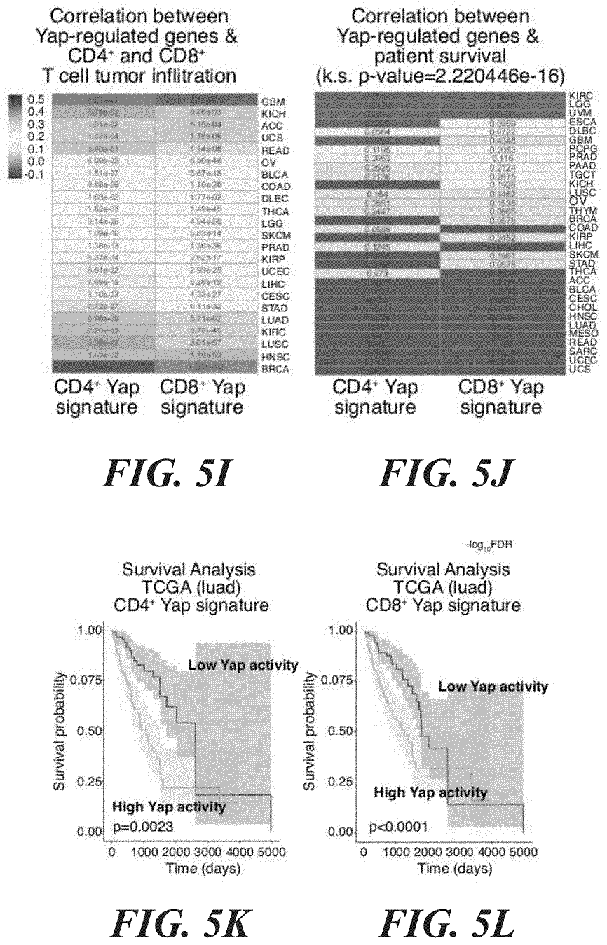

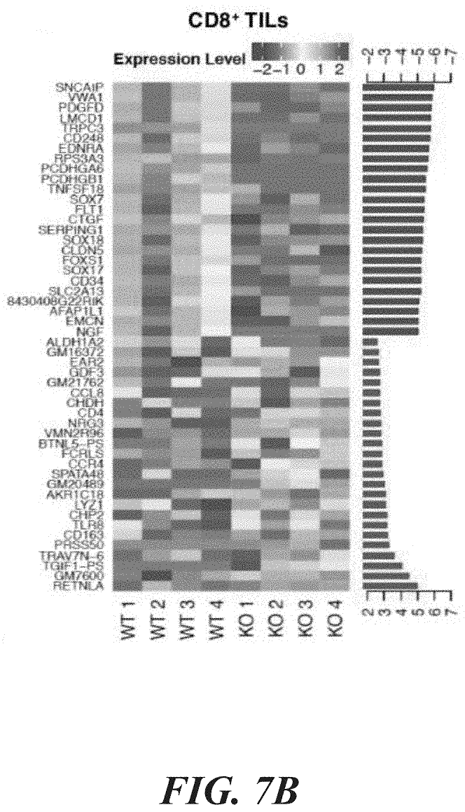

The inventors next aimed to define Yap-regulated signaling networks in T cells that impacted tumor growth and T-cell infiltration. CD4⁺ and CD8⁺ TILs were isolated from B16 tumors grown in WT or Yap-cKO mice, as well as CD4⁺ and CD8⁺ T cells from corresponding TDLNs and analyzed by RNA-seq (data not shown). A large number of genes were differentially expressed in CD4⁺ and CD8⁺ TILs isolated from Yap-cKO mice compared to WT mice (FIG. 5A, FIG. 5B, and FIG. 7A-7B). Notably, T cells from TDLNs showed markedly fewer gene expression changes with Yap deletion compared to TILs (FIG. 5C and FIG. 5D). These data are consistent with Yap function being coordinated with T-cell activation and show that the enhanced antitumor responses observed in Yap-cKO T cells are mediated by changes in cellular responses to local tumor signals, including TCR signaling and the cytokine milieu.

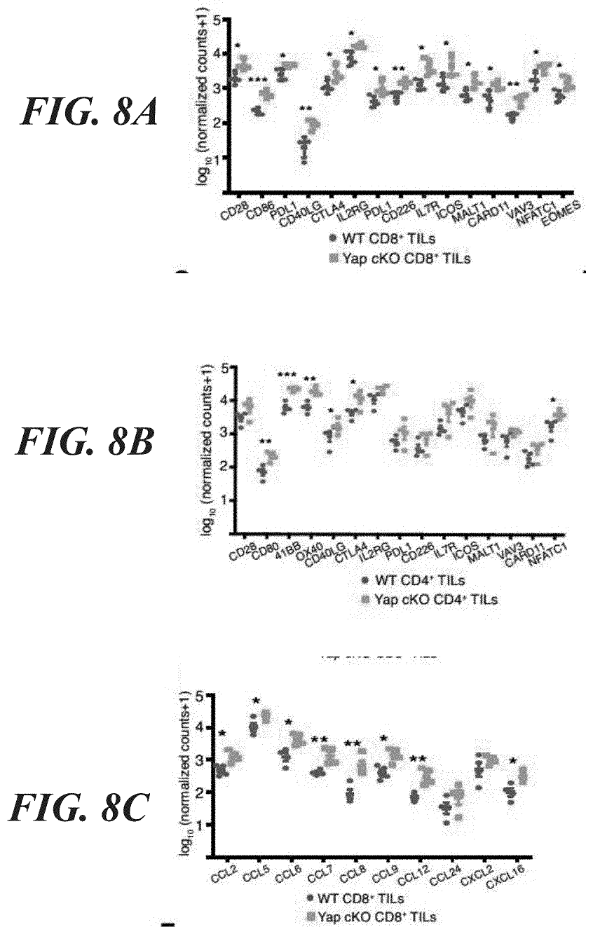

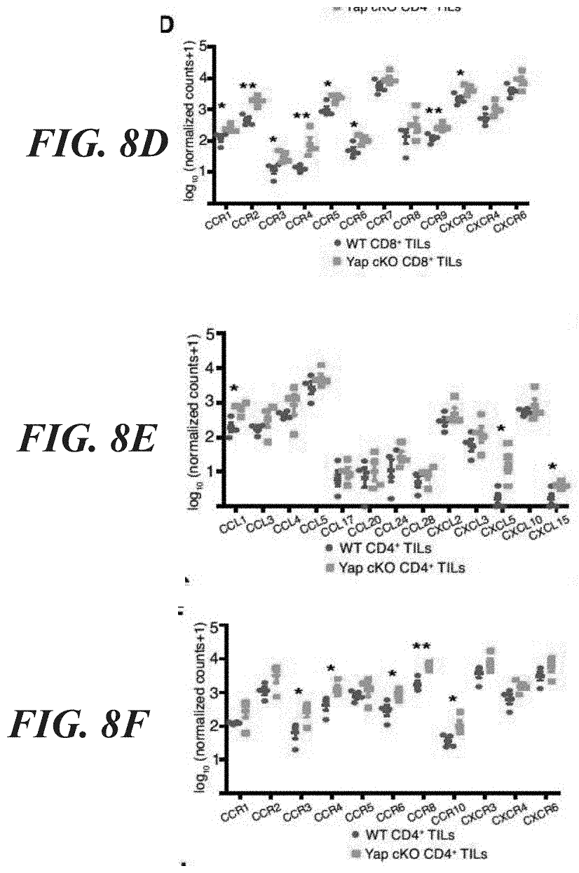

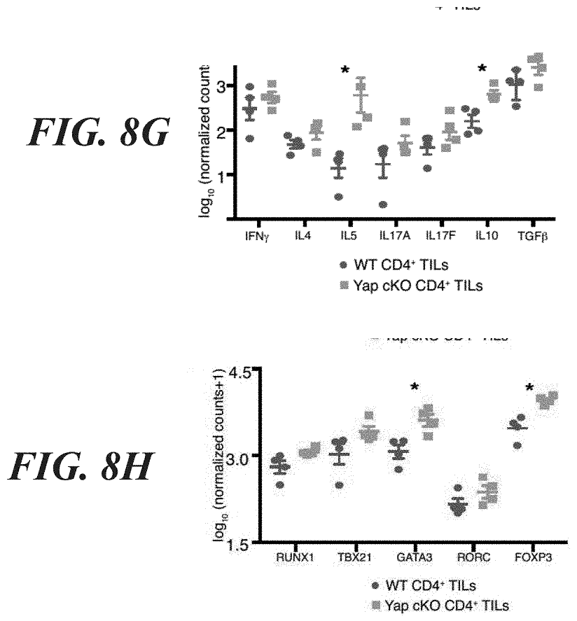

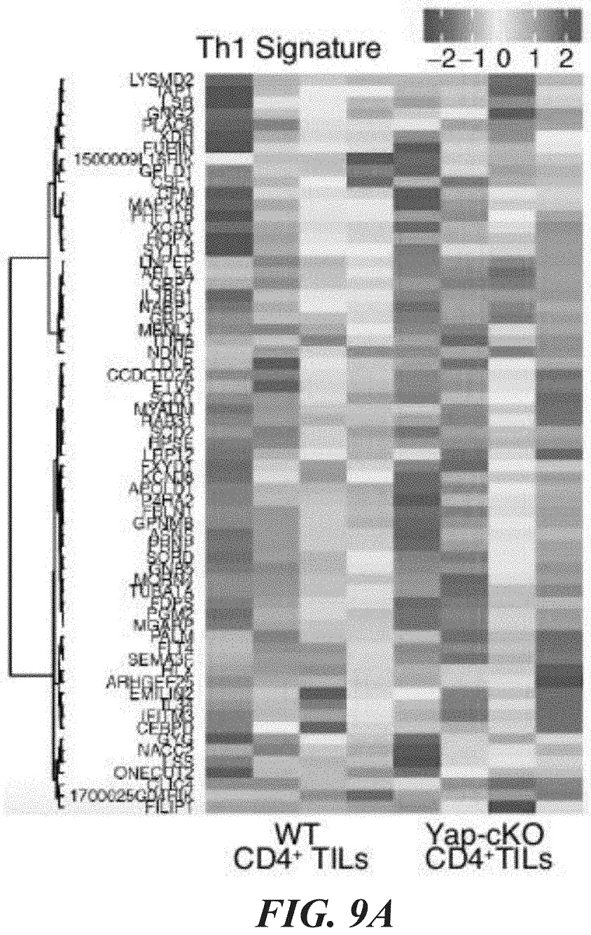

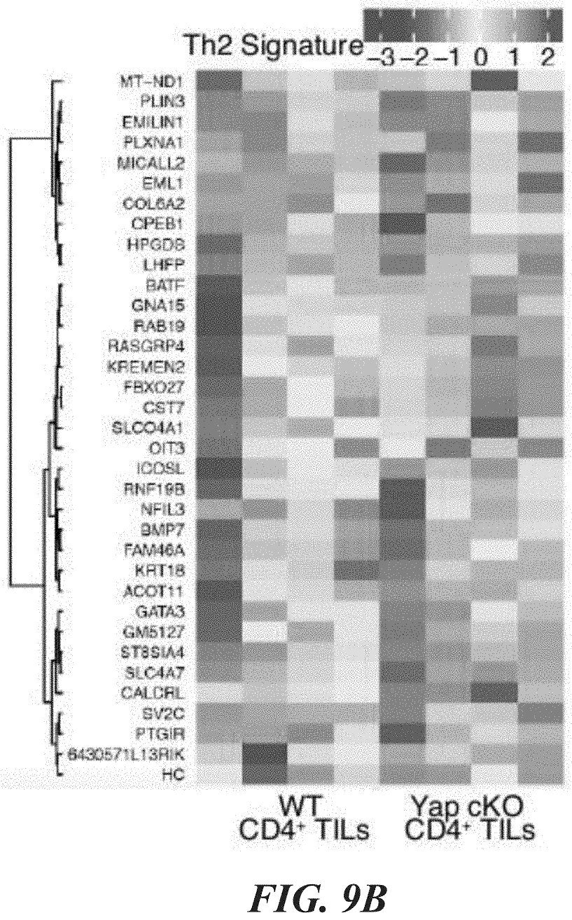

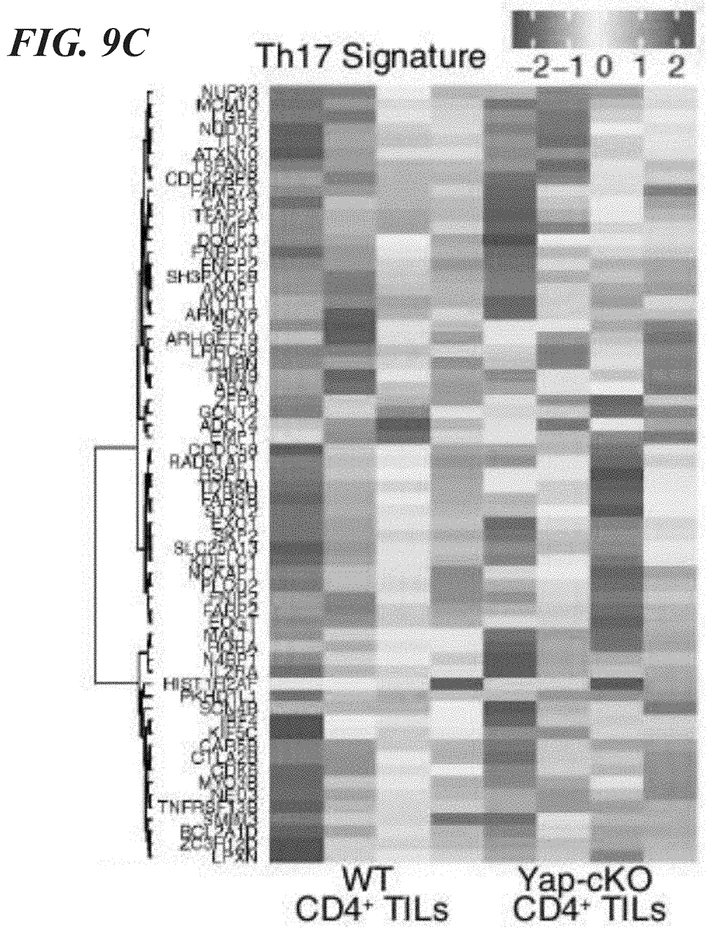

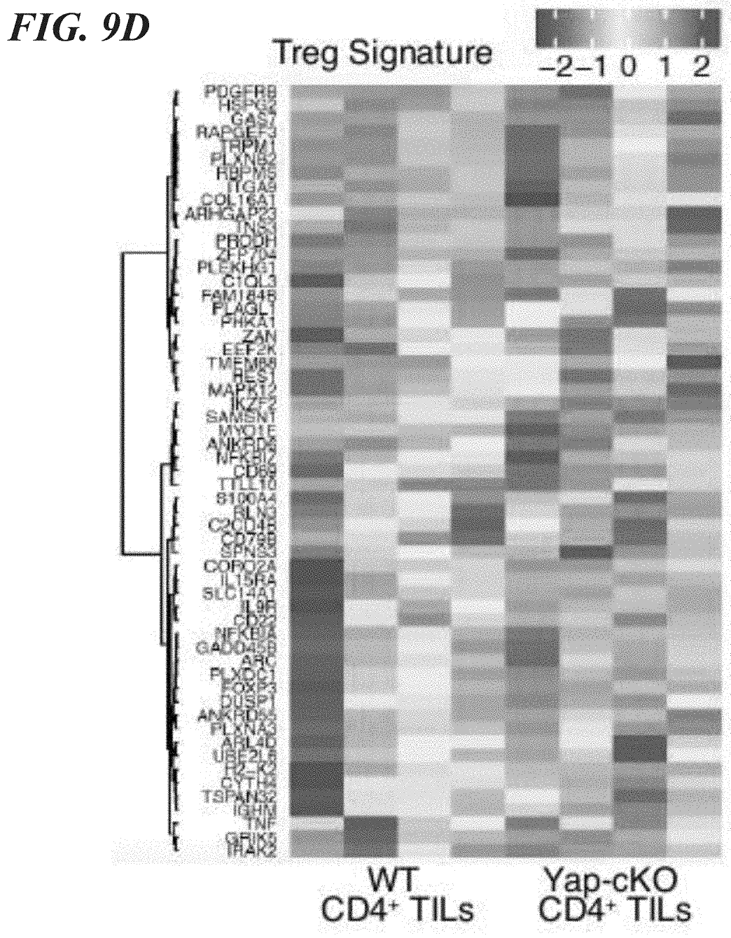

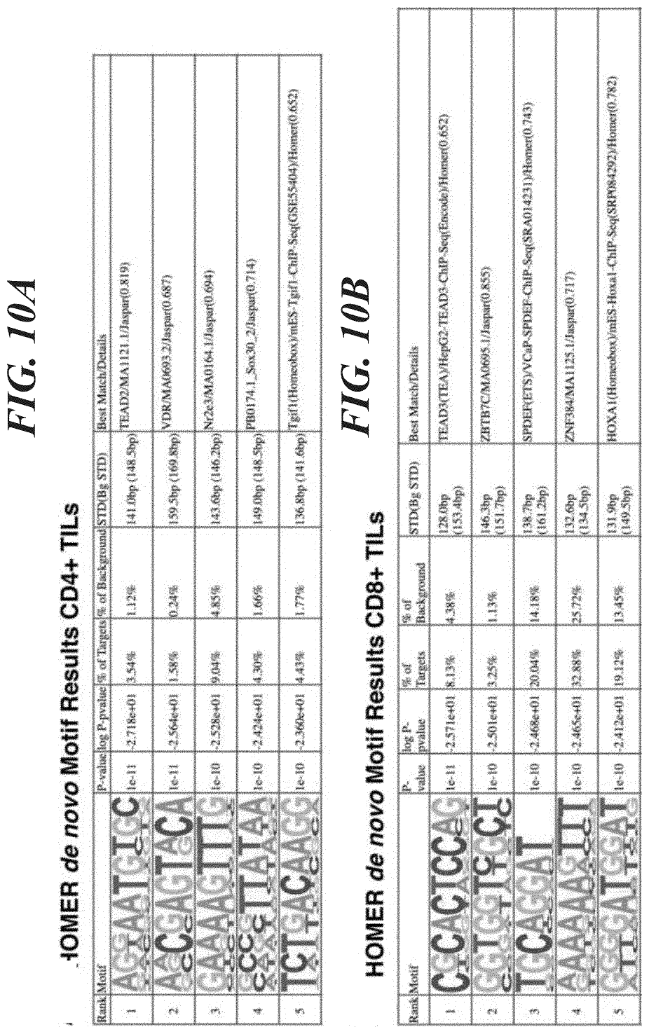

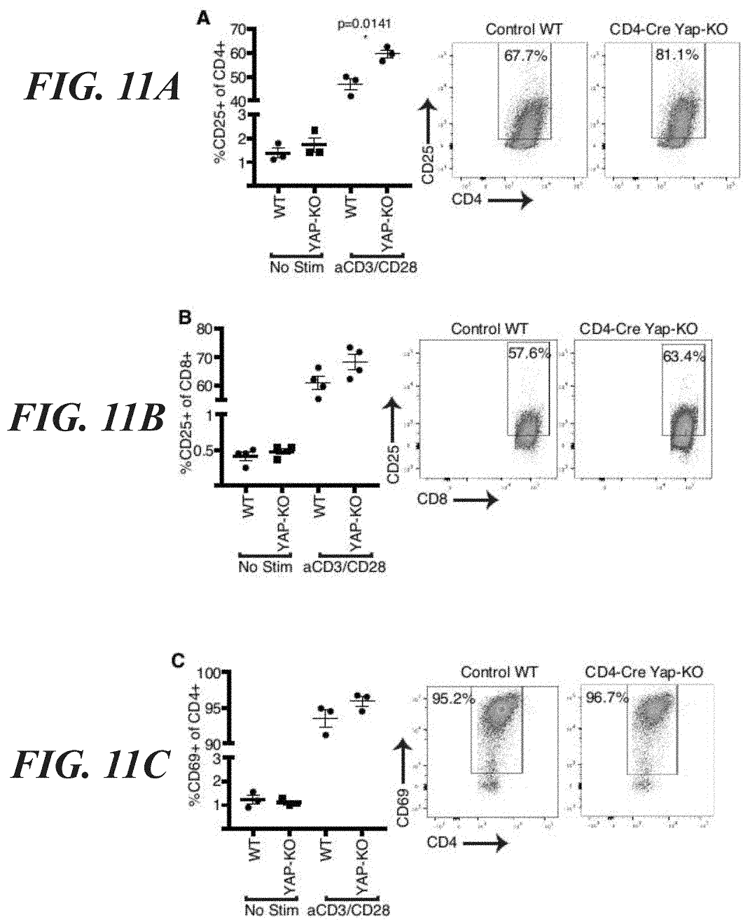

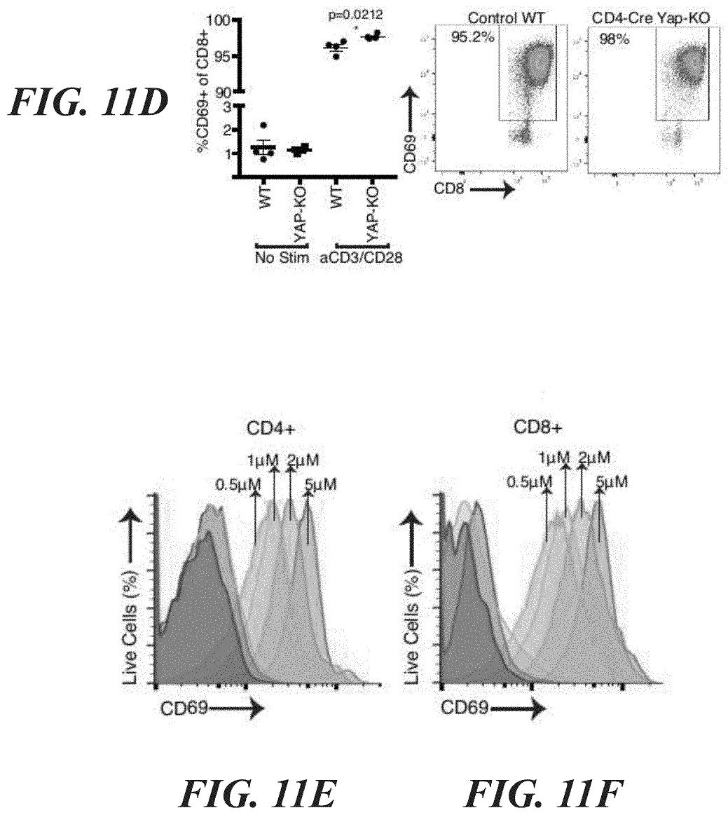

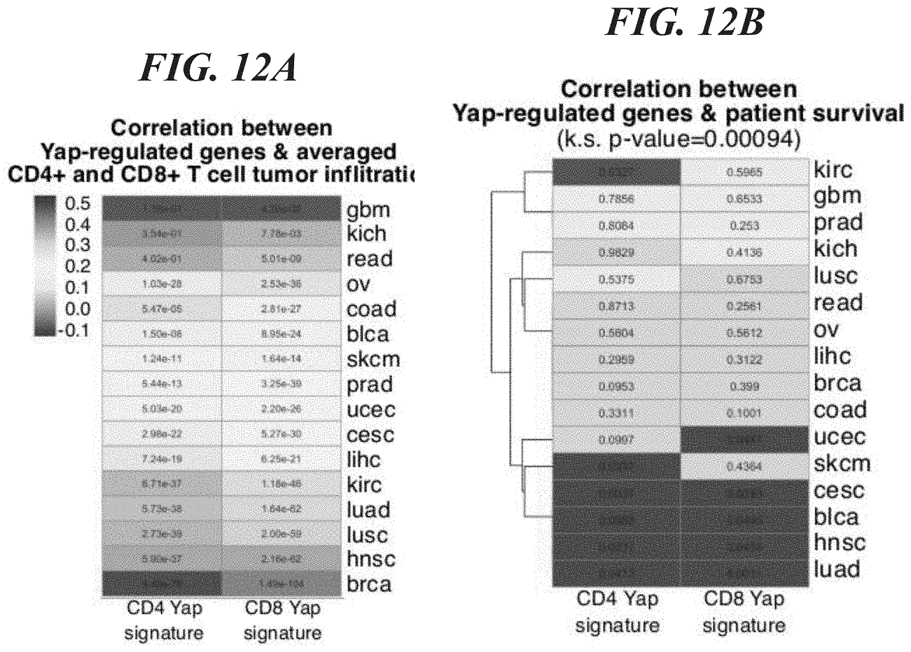

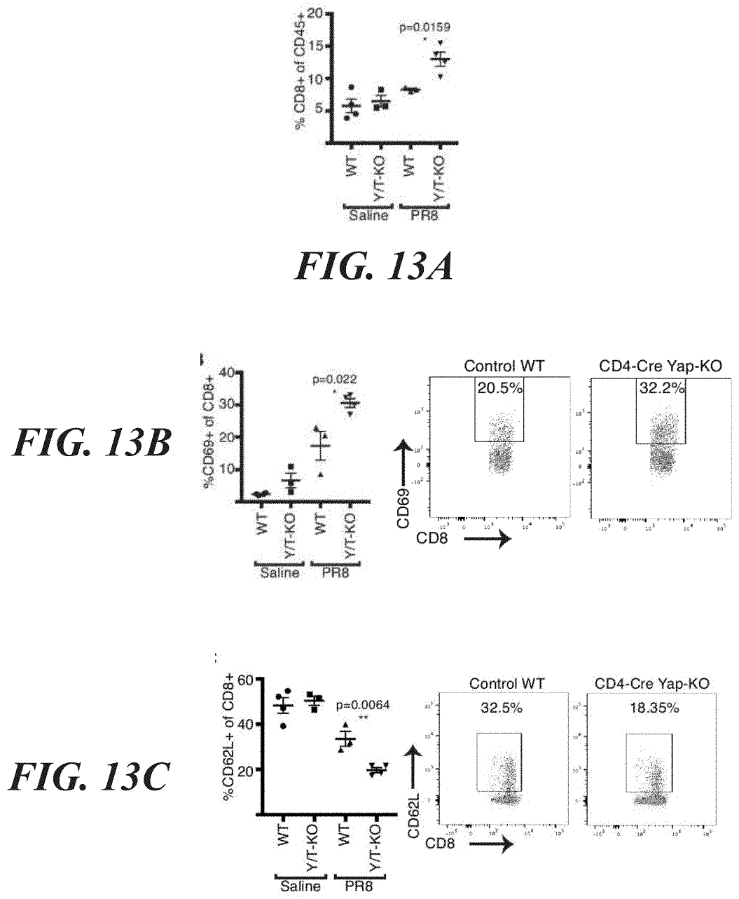

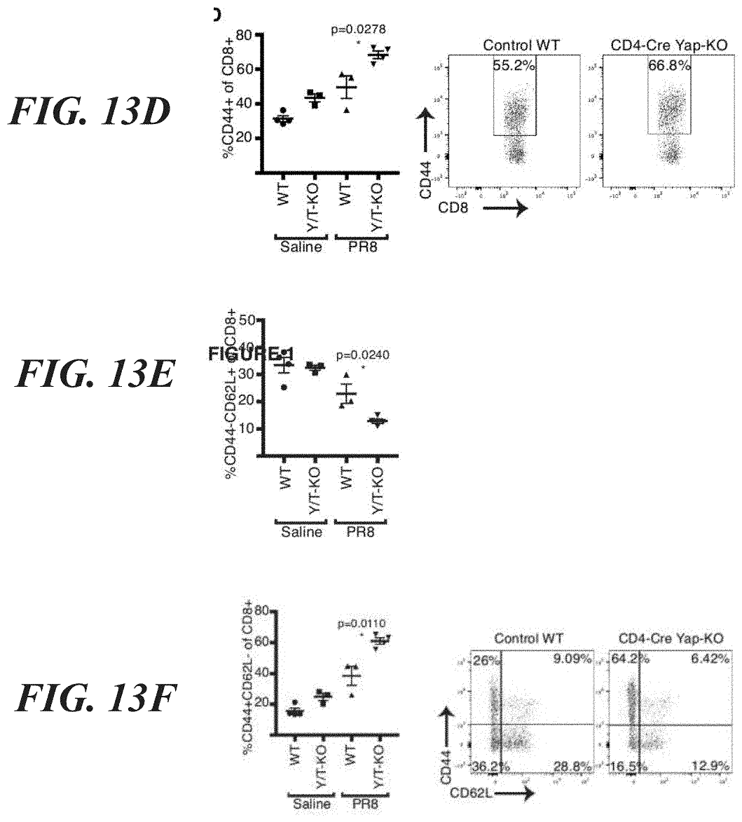

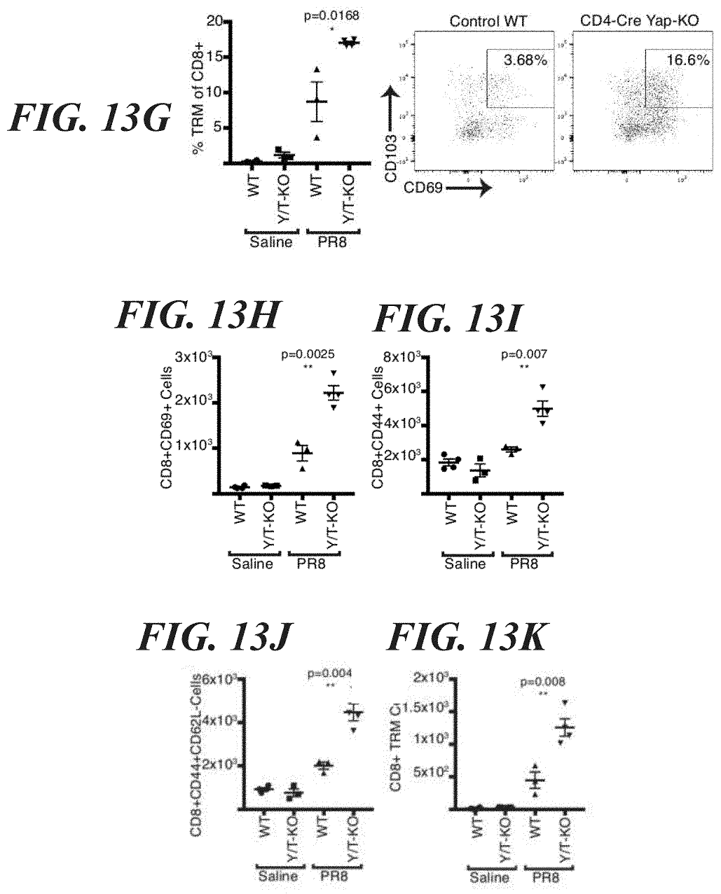

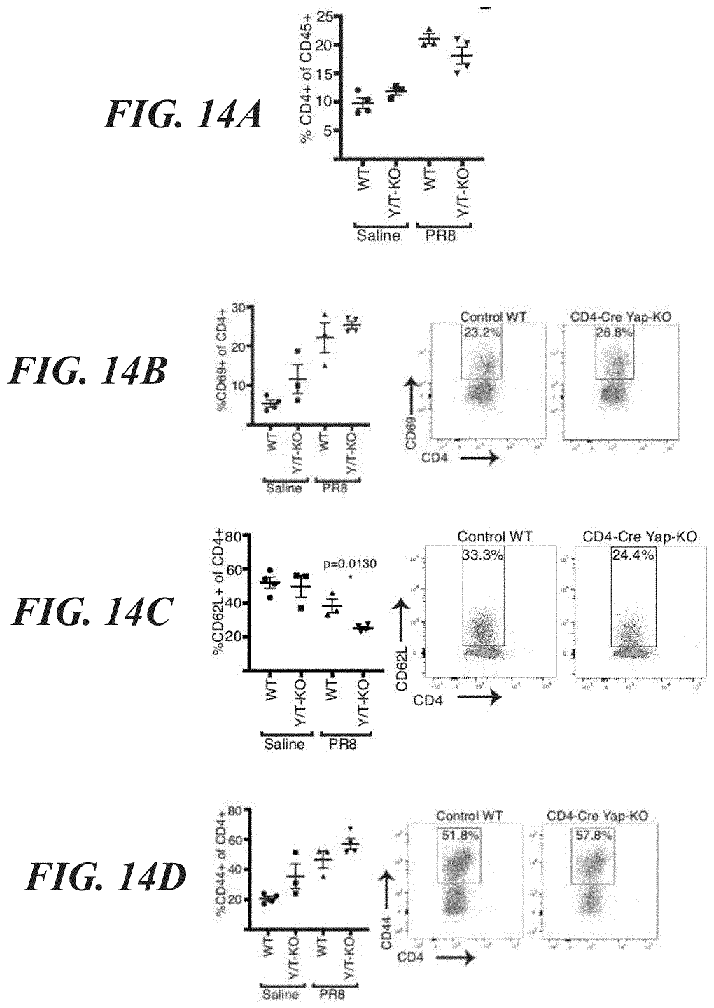

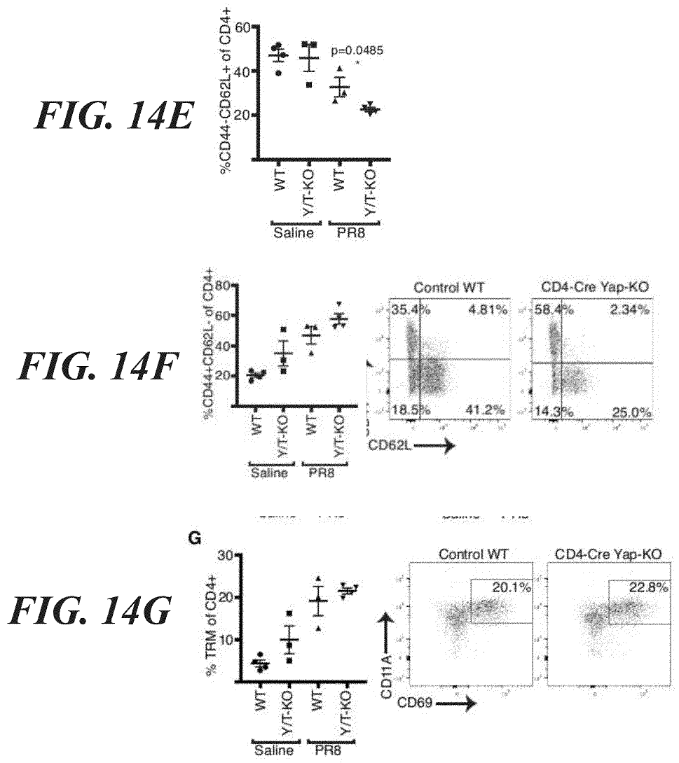

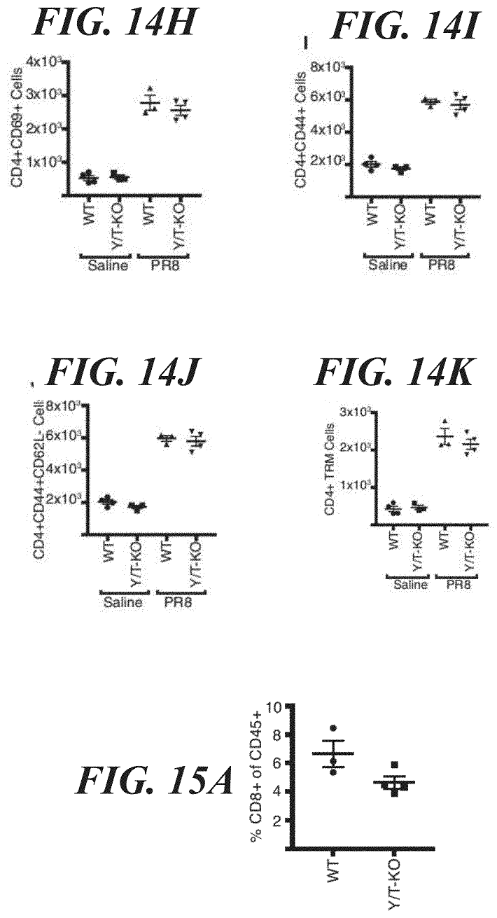

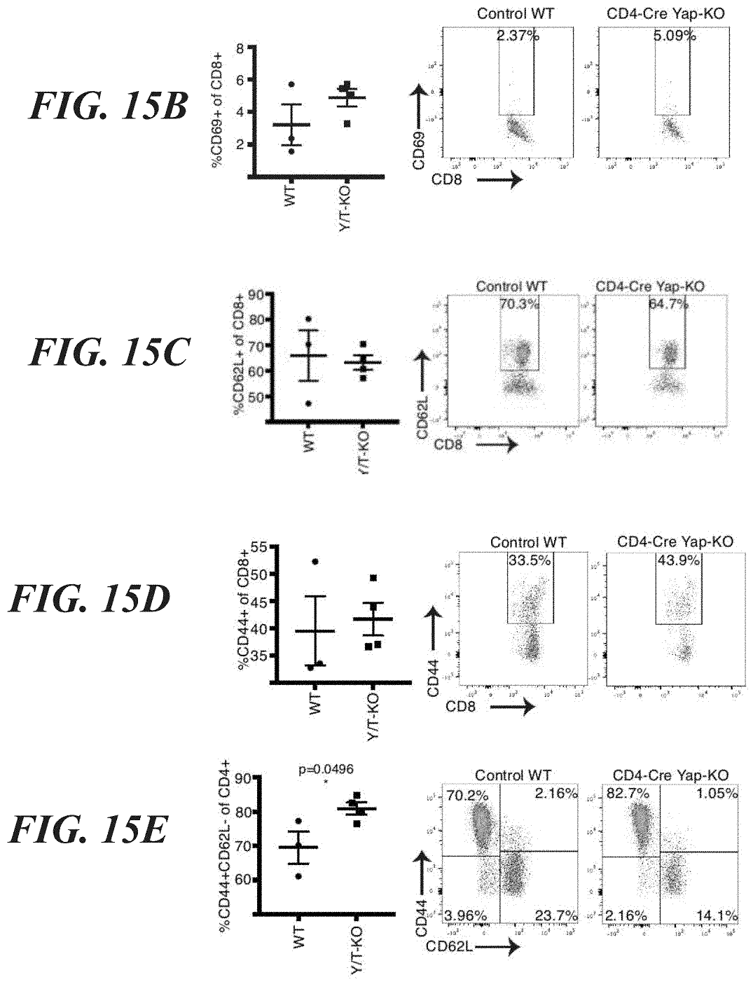

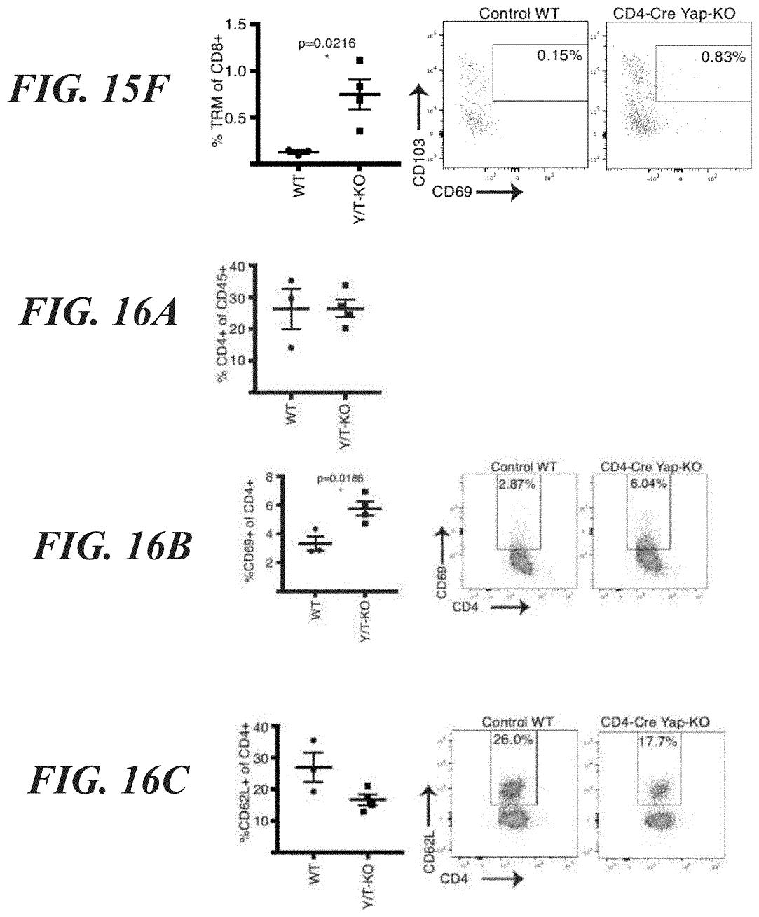

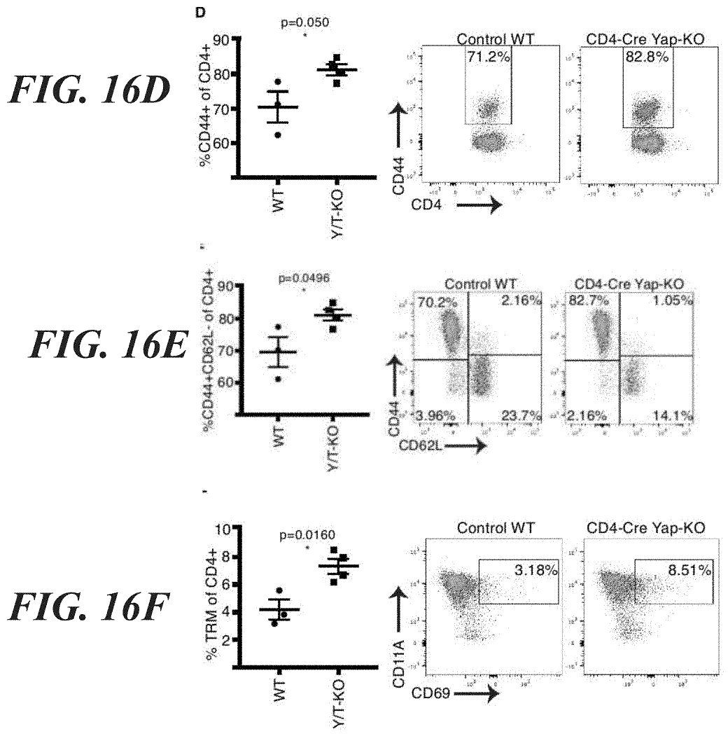

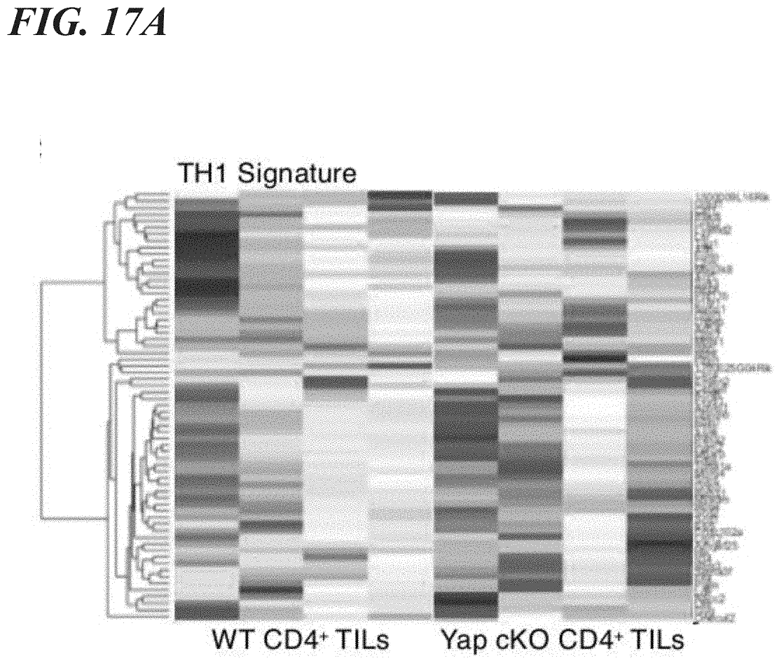

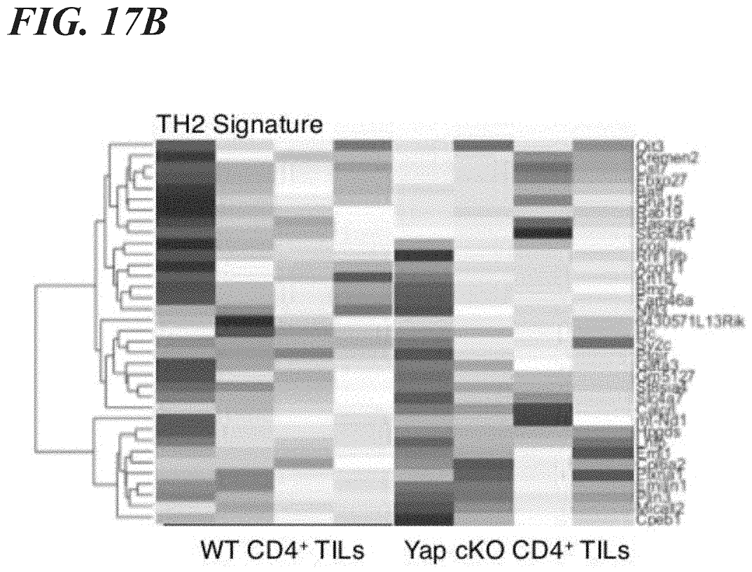

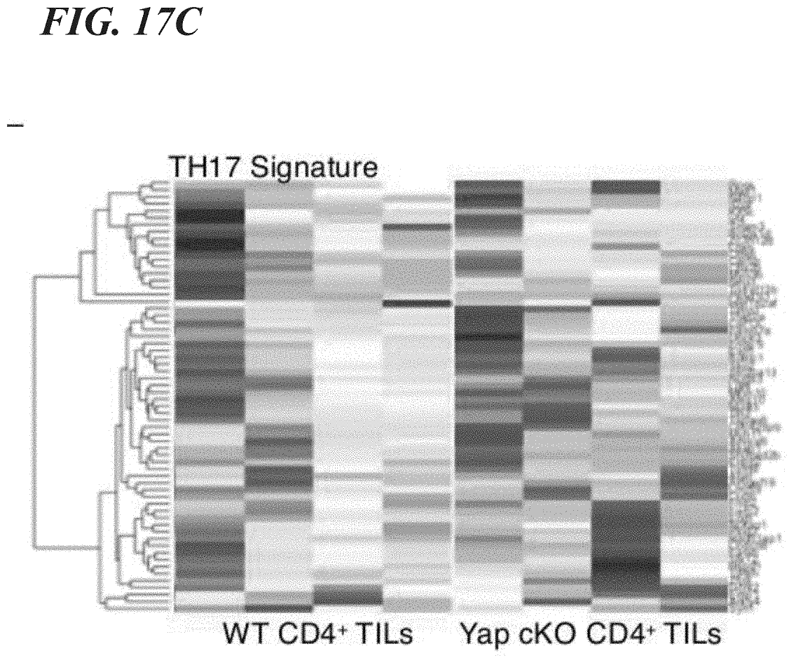

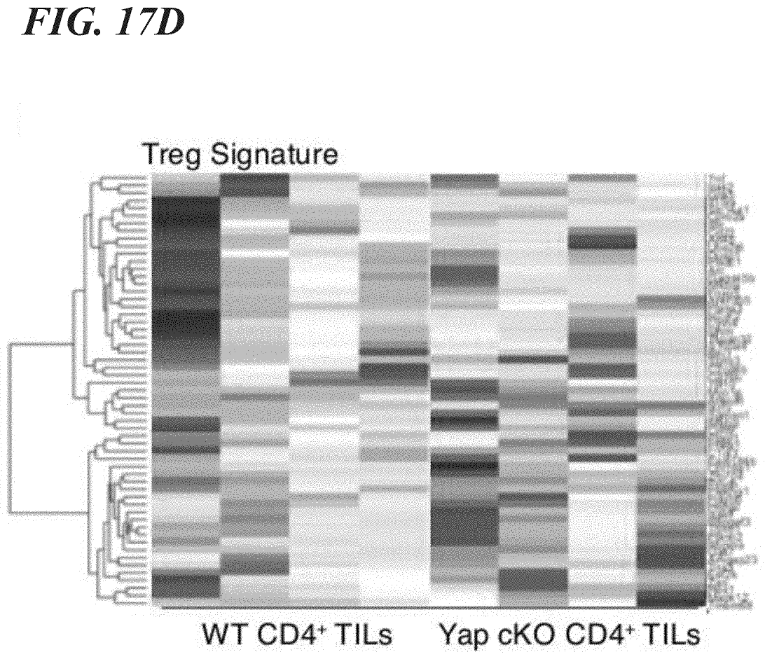

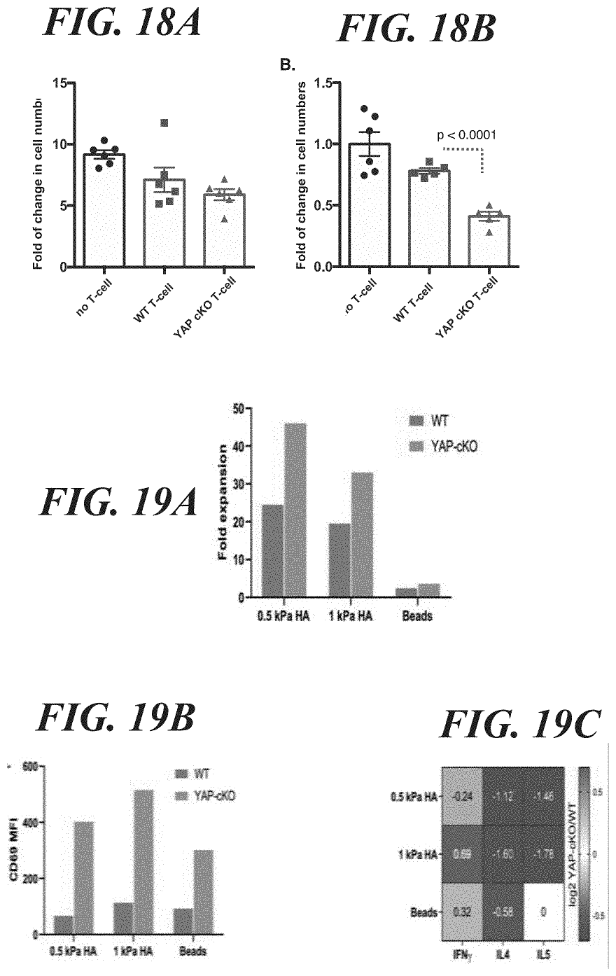

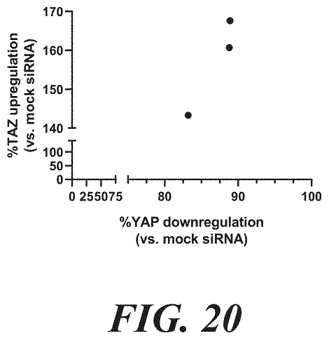

Hyper-enrichment analyses [63] of gene expression changes identified in TILs from Yap-cKO mice showed induction of genes related to T-cell activation, differentiation, survival, and migration (FIG. 5E and FIG. 5F) (Table 3), implicating Yap in fundamental T-cell processes beyond those previously reported. Yap-cKO TILs showed enrichment of genes associated with TCR signaling, genes that encode major costimulatory molecules, and genes downstream of TCR signaling (FIG. 8A and FIG. 8B), showing enhanced responsiveness to TCR signals in line with the inventors in vitro observations. Cytokine and cytokine receptor signaling gene sets were also enriched in Yap-cKO TILs, showing that cytokine production and responsiveness to cytokine receptor engagement were enhanced. Furthermore, chemokine and chemokine receptor signaling gene sets were enriched in Yap-cKO TILs (Table 3 and Fig C-F), which likely contributes to their improved tumor-infiltrating capacities. Subset-defining transcription factors and cytokines associated with each of the major CD4⁺ T-cell phenotypes were all up-regulated in Yap KO CD4⁺ TILs (FIG. 8G and FIG. 8H), showing that Yap deletion leads to enhanced naïve CD4⁺ T-cell differentiation, consistent with the inventors in vitro observations. Using unique up-regulated genes after differentiation to each of the major T helper subsets [64], the inventors data showed that Yap-cKO CD4⁺ TILs are more skewed towards a Th2 and Treg phenotype compared to WT CD4⁺ TILs (FIG. 9A-FIG. 9D), consistent with prior studies showing that the B16F10 tumor microenvironment enhances these fates [65-67]. As expected, gene set enrichment profiles identified in Yap-cKO TILs also included “Yap1 and TAZ stimulated gene expression” and “signaling by Hippo,” which were repressed in both CD4⁺ and CD8⁺ TILs (FIG. 5G and FIG. 5H). An unbiased analysis of transcription factor binding motifs in upstream regulatory regions of the genes altered in Yap-cKO CD4⁺ and CD8⁺ TILs revealed the TEAD transcription factor motif as the most significantly enriched in both cell populations (FIG. 10A and FIG. 10B), showing that Yap-regulated TEAD activity contributes to transcriptional regulation of these genes.

Comparison of gene expression changes identified in CD4⁺ and CD8⁺ Yap-cKO TILs with clinical data from TCGA [68] showed significant correlation of gene signatures with T-cell infiltration across a variety of human cancers (FIG. 5I, FIG. 12A). Consistent with the inventors preclinical results in melanoma (B16F10) and lung cancer (LLC), the inventors observed significant correlation with T-cell infiltration in skin cutaneous melanoma (SKCM), lung squamous cell carcinoma (LUSC), and lung adenocarcinoma (LUAD). Genes altered in Yap-cKO TILs were also significantly associated with patient survival across several cancers (FIG. 4J), as seen most significantly in LUAD for both CD4⁺ and CD8⁺ Yap-cKO gene signatures (FIG. 4K and FIG. 4L). These analyses showed that increased Yap activity in TILs correlated with poorer prognosis and lower T-cell infiltration, showing that Yap function in T cells contributes to aggressive human cancer development and cancer immunosuppression.

YAP Inhibits CD4+ and CD8+ Effector Memory and Tissue Resident Memory T Cell Generation.

Since the inventors showed that YAP affects primary CD4+ and CD8+ T cell responses, and that YAP loss amplifies the effects of microenvironmental signals during priming and differentiation, we hypothesized that combined YAP/TAZ deletion would increase the yield of CD8+ and CD4+ memory T cells during infection. We examined CD4+ and CD8+ effector memory (TEM) and resident memory (TRM) T cell generation in WT and YAP/TAZ-cKO (Yap-loxP/loxP; Wwtr1-loxP/loxP; CD4-Cre) mice using two in vivo infection models: a viral infection with influenza (flu; strain PR8), and a bacterial infection with S. pneumoniae, serotype 19 (SP19).

In the flu model, T cell phenotypic analysis of lungs from PR8 infected mice 49 days post infection revealed that YAP/TAZ-cKO leads to generation of significantly more CD8+ TEM and lung TRM cells compared to WT mice (FIGS. 13A-13K). CD8+ T cell infiltration to the site of the lung as a fraction of CD45+ cells in the lung was increased in the YAP/TAZ-cKO mice compared to WT (FIG. 13A). Consistent with our observations in YAP-cKO mice, CD69 expression in YAP/TAZ-cKO CD8⁺ T cells was substantially higher than in the WT (FIG. 13B). Naïve T cell marker CD62L was repressed and memory marker CD44 was induced in YAP/TAZ-cKO T cells (FIG. 13C and FIG. 13D). We observed that the percentage of CD44−CD62L+CD8+ lung infiltrating T cells decreased in YAP/TAZ-cKO mice while the percentage of CD44+CD62L−CD8+ TEM as well as CD44+CD62L-CD103+CD69+CD8+ TRM increased significantly compared to WT (FIGS. 13E-13G). Importantly, the number of CD8+CD69+, CD8+CD44+, CD8+CD44+CD62L− TEM, as well as CD8+CD44+CD62L−CD11A+CD69+ TRM cells increased dramatically in the YAP/TAZ-cKO mice compared to the WT (FIGS. 13H-13K). Such changes were not observed in the CD4+ T cell compartment (FIGS. 14A-14F). Although we observed that the percentage of naïve CD4+ T cells decreased in YAP/TAZ-cKO mice, there were no changes with expression of CD69, or CD4+ TEM or TRM formation (FIG. 14A-14K). Interestingly, during a pneumococcal infection, we observed no differences in the extent of CD8+ TEM or TRM generation, but there was a significant increase in CD69 and CD44 expression in CD4+ T cells in YAP/TAZ-cKO mice, repression of CD62L and increased CD4+ TEM and TRM generation (FIGS. 15A-15F and FIGS. 16A-16F). Collectively, these data show that YAP (and TAZ) play important roles in the regulation of memory T cells, and that repression of YAP/TAZ activity may be used as a mechanism for increasing memory cell numbers and function.

Discussion

The inventors present evidence for an inhibitory role for Yap in both CD4⁺ and CD8⁺ T cells and offer the first data showing that Yap is a negative regulator of T-cell tumor infiltration. The inventors found that disrupting Yap activity leads to enhanced T-cell activation, augmented differentiation, and increased tumor infiltration. Further, the inventor's data show that inhibition of YAP during the process of generating T cells for adoptive cell transfer improves immune responses and clinical outcome. Further, given that loss of YAP promotes memory markers in CD4+ and CD8+ T cells, incorporating YAP inhibition in vaccination regimes will also improves vaccine efficacy. Two major signals are necessary for T-cell activation: signaling from engagement of the TCR with its cognate antigen:MHC complex and a second signal from the costimulatory receptor CD28 binding to ligands CD80 and CD86 [69,70]. Experimental data showed that CD4⁺ and CD8⁺ T cells from Yap-cKO mice were more sensitive to TCR signaling compared to WT cells. The inventors demonstrated significant up-regulation in sensitivity of activation markers CD44, CD25, and CD69 to TCR signal strength. Through recruitment of key effector kinases, phosphatases, and scaffold proteins, transcriptional programs are induced by TCR signaling that leads to production of cytokines and cytokine receptors, which include IL-2 and CD25, the a chain of the heterotrimeric high-affinity IL-2 receptor. IL-2 binding to the IL-2 receptor, together with TCR signaling and costimulation, elicit transcriptional changes resulting in proliferation and differentiation [71]. RNA-seq analysis ofYap-cKO versus WT TILs revealed that CD3, CD28, CD80, and CD86 receptor molecules, kinases, phosphatases, and scaffold proteins are all up-regulated with Yap deletion, offering a mechanism for enhanced activation of CD4⁺ and CD8⁺ T cells. These mechanisms are significantly broader in scope than the TGFβ-specific mechanisms previously reported for Yap in Tregs [37], implicating Yap as a regulator of all activated T-cell responses.

Our analysis of thymic populations provides the first evidence that Yap deletion does not cause significant changes in thymocyte maturation. These data show that Yap functions primarily in mature T cells, and loss of Yap is not deleterious to thymocytes. While the precise reason for differential action of Yap in thymocytes versus mature T cells remains undefined, other proteins have distinct TCR-induced mechanisms in immature thymocytes compared to mature T cells. For example, Nur77 is less pro-apoptotic in mature T cells compared to thymocytes due to differences in TCR-induced phosphorylation and kinase activity [72]. The inventors observed a slight increase in total number of TCRβ⁺CD69⁺ thymocytes in Yap-cKO mice compared to WT mice. This increase in TCR signaling post positive selection is consistent with our observations that Yap enhances T-cell sensitivity to TCR signaling. Interestingly, the inventors did not see a corresponding increase in numbers of Yap-cKO T cells at later maturation stages, showing that the increased numbers of positively selected thymocytes may not survive negative selection. Overall, these data show that Yap-cKO mice are developmentally similar to WT mice. However, our studies do not rule out the potential that Yap may act by subtly shifting the specificity of the T-cell repertoire during development, which may contribute to some of the effects observed in our work. This remains an important area for future studies.

The inventors demonstrated that Yap-cKO enhanced CD4⁺ and CD8⁺ T-cell activation in vitro. Observed increases in TEAD1 and TEAD3 expression in activated T cells show that Yap may exert immunosuppressive effects through TEAD-regulated transcription. Consistent with this idea, TEAD-binding motifs were the most enriched motif in the Yap-regulated gene expression signature identified in Yap-cKO TILs. Furthermore, treatment with verteporfin, a reported inhibitor of Yap-TEAD activity [73], increased T-cell activation similar to Yap deletion. Interestingly, neither Yap deletion nor verteporfin treatment significantly impacted T-cell proliferation. Previous studies have demonstrated that proliferation has a sharp, switch-like threshold for TCR signal to elicit proliferation [74], and thus it is likely that our experimental conditions provided optimal signaling above this threshold. Expression levels of activation markers have been directly linked to TCR signal strength, such as CD69 levels being directly regulated by affinity and dose of TCR ligand [75] and CD71 levels being directly linked to mammalian target of rapamycin (mTOR) activation level [76]. Our observations therefore support a novel conceptual advance, which is that Yap may link TCR signal strength to negative feedback.

The rapid induction of Yap post T-cell activation implicates shared canonical signals of T-cell activation in the regulation of Yap. These observations are consistent with those made previously in naïve CD8⁺ T cells [27] but contrast those arguing Treg-specific roles for Yap [37]. Our data show that Yap promotes a normal negative feedback mechanism during T-cell activation similar to inhibitory checkpoint molecules, and inhibition of Yap must be timed before or during T-cell activation. Our observations interestingly also show that Yap plays a prominent role only following activating signals from the microenvironment. This is highlighted by the large number of genes impacted in Yap-deleted CD4⁺ and CD8⁺ T cells isolated from TILs compared to few genes differentially expressed in TDLNs, and it is consistent with our data showing that Yap levels increase after T-cell activation. These data show that therapeutic inhibition of Yap in T cells may have fewer side effects compared to strategies such as checkpoint blockade, since the regulatory activity of Yap is synchronized with T-cell activation and appears specific to sites of active T-cell priming. Yap connects a variety of extracellular stimuli into intracellular cues that inform the cell of its own structural features (actin cytoskeleton, polarity, cell shape) as well as its location and surroundings (mechanical, adhesion, extracellular matrix), instructing cellular survival, proliferation, differentiation, and fate. Therefore, a better understanding of how Yap is regulated by these signals in T cells may reveal new insights into immunoregulatory mechanisms.

Regulation of T-cell activation by the microenvironment through polarizing cytokines allows for diverse, context-specific differentiation and functional diversity in CD4⁺ T cells [3]. The inventors observe that Yap-cKO CD4⁺ T cells have increased capacity to differentiate towards Th1, Th2, Th17, and Treg phenotypes. These observations show that Yap does not preferentially bias control of T-cell differentiation but instead enhances responsiveness to local microenvironment cues. These data therefore show that Yap inhibition may enhance responses to current immunotherapy strategies. Clues into Yap-regulated events are embedded in our RNA-seq of Yap-cKO TILs, which show broad changes in T-cell responses. Genes regulated by Yap include several key effectors of T-cell activation, including genes involved in nuclear factor kappa-light-chain-enhancer of activated B cells (NFkB), mitogen-activated protein kinase (MAPK), nuclear factor of activated T cells (NFAT), and Janus kinase (JAK)-signal transducers and activators of transcription (STAT) signaling. Notably, Yap-regulated genes are also enriched for those regulated by the TGFβ and Wnt pathways, which have important pleotropic roles in T-cell biology [77,78]. Given the known convergence of Yap with these immunomodulatory pathways [79], it is likely that Yap directs their transcriptional targets and signal strength. Our study provides novel evidence of CD4⁺ and CD8⁺ T-cell-intrinsic effects of Yap on T-cell activation and tumor infiltration.

An important finding of our studies is that Yap-cKO mice show delayed tumor growth in conjunction with increased infiltration and expression of effector molecules. The well-characterized B16F10 melanoma and LLC lung cancer models generate tumors that are characterized as immune deserts [54-57, 80-82]. In immune deserts, T cells are completely excluded from the tumor microenvironment due to suppressed tumor immunogenicity and insufficient T-cell priming, costimulation, and activation [24]. The ability for Yap-cKO CD4⁺ and CD8⁺ T cells to become activated and infiltrate tumors that normally inhibit T-cell infiltration is therefore remarkable and important, as these phenotypes are key for combating tumor growth in human cancer patients [83]. Adoptive transfer of Yap-cKO CD8⁺ T cells showed that CD8⁺ T cells have intrinsically enhanced tumor infiltration capacity compared to WT host CD8⁺ T cells into B16F10 tumors. These data are novel and show that one of the early events in Yap-mediated tumor immunosuppression may be exclusion of CD8⁺ T cells from the tumor. The significant correlation of CD8⁺ and CD4⁺ Yap-cKO gene signatures with tumor T-cell infiltration in TCGA data show that Yap represses CD8⁺ and CD4⁺ T-cell migration and tumor infiltration in human cancers. In particular, our preclinical results in melanoma and lung cancer models were mirrored in human SKCM, LUAD, and LUSC. Low activity of Yap-regulated CD4⁺ and CD8⁺ gene expression signatures correlated with immune infiltration in 19 TCGA cancer types—and strongly correlated with survival in 11 TCGA cancer types-highlighting that Yap activity in T cells has broad potential as a target for cancer therapy.

Collectively, the inventor's data show that Yap is an important immunosuppressor and show that inhibition of Yap activity in T cells have important clinical implications in T-cell therapies against cancer, autoimmune and infectious disease. The inventors show for the first time that Yap is expressed in activated CD4⁺ and CD8⁺ T cells and plays a regulatory role in T-cell activation for both subsets.

Materials and Methods

Ethics Statement

This study was performed in strict accordance with the recommendations in the Guide for the Care and Use of Laboratory Animals of the National Institutes of Health. Animal care and handling was consistent with the recommendations of the Panel on Euthanasia of the American Veterinary Medical Association. Prior to the initiation of experiments, all study protocols were reviewed and modified according to the show ions of the Boston University School of Medicine IACUC. The Boston University School of Medicine animal management program is accredited by the American Association for the Accreditation of Laboratory Animal Care and meets National Institutes of Health standards as set forth in the Guide for the Care and Use of Laboratory Animals (DHHS Pub. No. [NIH] 85-23, rev 1985). Boston University's Animal Welfare Assurance number is A-3316-01.

Mouse Strains and Genotyping

Yap-loxP/loxP mice, provided by Dr. Jeff Wrana and previously described [84], were backcrossed to the C57BL/6 background for 10 generations and bred with the Tg(Cd4-cre)1Cwi (Jax: 022071) [85] and LSL-EYFP (Jax: 006148) [86] lines to derive Yap-loxP/loxP; LSL-EYFP; CD4-Cre mice. For adoptive cell transfers, CD4-cre mice were crossed with LSL-tdTomato mice (Jax: 007914) [87]. All experiments were performed with 6- to 10-week-old mice, and littermates were always used as controls for each experiment. Animal protocols and study designs were approved by Boston University School of Medicine and UMBC. Mice were maintained in pathogen-free facilities at BUMC and UMBC and were PCR genotyped using published protocols [84-87].

Cell Culture and Mouse Tumor Challenges

B16F10 mouse melanoma cells (ATCC CRL-6475) and LLC1 cells (ATCC CRL-1642) were cultured in DMEM supplemented with glucose, L-glutamine, sodium pyruvate, 10% FBS, penicillin, and streptomycin. Cells were split once they reached 70% confluency and were not used for mouse challenge past a fifth passage. T cells were cultured in RPMI 1640 supplemented with 10% FBS, 1 mM sodium pyruvate, 50 μM β-ME, penicillin, streptomycin, 2 mM L-glutamine, 100 mM nonessential amino acids, 5 mM HEPES, free acid and 3-mercaptoethanol. For tumor inoculations, 5×10⁴ B16F10 cells or 5×10⁵ LLC cells were injected subcutaneously on the right flank of each mouse on day 0. Tumor volume was estimated using the formula (L×W²)÷2. Survival endpoint was reached once the tumors measured 500 mm³, around day 15. Mice were euthanized by isoflurane inhalation and subsequent cervical dislocation, and tumors were harvested for further prospecting.

T-Cell Isolation

Spleens from WT or Yap-cKO mice were pushed through a 70 μm mesh (Falcon) using an insulin syringe plunger and washed with PBS. Cells were treated with ACK red blood cell lysis buffer, and splenocyte single-cell suspensions were prepared for magnetic separation or stained for sorting by flow cytometry. CD4⁺ or CD8⁺ T-cell enrichment was performed using magnetic beads (Miltenyi Biotec or STEMCELL Technologies). Naïve CD4⁺ T cells were isolated using a naïve CD4⁺ T-cell isolation kit (Miltenyi Biotec).

Flow Cytometry

Isolated splenocytes or tumor digests were washed with PBS and stained with the LIVE/DEAD fixable near-IR dead cell stain kit (Invitrogen). Cells were then washed with stain buffer (BD), resuspended in stain buffer containing Fc block (BD), and incubated for 5 minutes at 4° C. Surface antibodies were added in predetermined concentrations, and cells were incubated for 30 minutes at 4° C. or 15 minutes at room temperature, before being washed with BD stain buffer and resuspended in PBS for flow cytometric analysis. For intracellular cytokine staining, cells were fixed and permeabilized using the BD Cytofix/Cytoperm Fixation/Permeabilization kit (BD) after dead cell dye and surface staining. For transcription factor staining, the eBioscience Foxp3/transcription factor staining buffer set was used. Flow cytometry analyses were performed on BD LSRII at Boston University School of Medicine Flow Cytometry Facility or at the University of Maryland School of Medicine Center for Innovative Biomedical Resources, Flow Cytometry Shared Service and analyzed by Flowlo (TreeStar).

T-Cell Activation and Proliferation Assays

Cells were cultured and stimulated in 96-well plates at 1×10⁵ cells per well. Plates were coated with anti-CD3 antibody (Biolegend) at concentrations of 0, 0.125, 0.25, 0.5, and 1 μg/ml at 4° C. overnight and were washed twice with PBS before incubation. Cells were stimulated in the anti-CD3-coated plates with soluble anti-CD28 at 2 μg/ml (Biolegend). On days 1 and 3, cells were stained with dead cell dye as well as antibodies recognizing the lineage and activation markers CD3 BUV737 (BD), CD4 BUV395 (BD), CD8 PerCP-Cy5.5 (Biolegend), CD69 PE (Biolegend), CD44 BV650 (Biolegend), and CD25 APC (Biolegend). For proliferation assays, CD4⁺ or CD8⁺ T cells were isolated and stained using the CellTrace Violet or CFSE Cell Proliferation Kit (Life Technologies). Briefly, purified cells were washed with PBS and incubated with CellTrace dye for 20 minutes at 37° C. protected from light. After 20 minutes, complete RMPI medium was added to the cell suspension, and the cells were incubated 5 minutes further before being washed and resuspended in complete RPMI medium. Cells were cultured in 96-well plates at 1×10⁵ cells per well and were stimulated using anti-CD3/CD28 dynabeads (Gibco) at a 1:1 ratio with T cells. On days 1 and 3, cells were stained with dead cell dye, and proliferation was measured at the same time.

CD4⁺ T-Cell In Vitro Differentiation

For CD4⁺ T-cell in vitro differentiations into Th1, Th2, Th17, and Treg subsets, naïve CD4+ T cells were enriched using magnetic beads (Miltenyi Biotec). Purified cells were plated at 1×10⁵ cells per well on a 96-well plate coated with 10 μg/ml anti-CD3 (Biolegend) and cultured with 2 μg/ml soluble anti-CD28 (Biolegend). The following conditions were specific to each differentiation regime: Th1: 10 ng/ml IL-12 and 10 μg/ml anti-IL-4; Th2: 50 ng/ml IL-4, 10 μg/ml anti-IFNγ, and 10 μg/ml anti-IL-12; Th17: 50 ng/ml IL-6, 20 ng/ml IL-1β, 5 ng/ml IL-23, 1 ng/ml TGFβ, 12 μg/ml αIFNγ, and 10 μg/ml anti-IL-4; and Treg: 100 IU/ml IL2, and 0, 0.5, or 5 ng/ml TGFβ. Cytokines and antibodies were purchased from Biolegend, except for IL-2 and TGFβ (R&D). Cells were cultured for 5 days, before being stimulated with 50 ng/ml PMA (Sigma, P1585) and 1 μg/ml ionomycin (Sigma, 10634) for 6 hours at 37° C. in the presence of Golgistop (monensin, BD) or Golgiplug (brefeldin, BD) added after the first 30 minutes of stimulation. Cells were stained with the LIVE/DEAD fixable near-IR dead cell stain kit (Invitrogen); antibodies for surface markers CD3 BUV737 (BD), CD4 BUV395 (BD), CD8 APCFire750 (Biolegend), and CD25 BV510 (Biolegend); antibodies for intracellular cytokines IFNγ APC (Biolegend) and IL-17 PerCP-Cy5.5 (Biolegend); or antibodies for transcription factors GATA3 PECy7 (Biolegend) and Foxp3 PE (BD), as described earlier.

Thymocyte Phenotyping

Thymuses from WT or Yap-cKO mice (8-10 weeks old) were mechanically disrupted by being pushed through a 70 m mesh (Falcon) with an insulin syringe plunger and washed with PBS. Cells were treated with ACK red blood cell lysis buffer, and thymocyte single-cell suspensions were stained for analysis by flow cytometry as described earlier. Cells were stained with dead cell dye and antibodies for the following surface markers: CD3 BUV737 (BD), CD4 BUV395 (BD), CD8 PerCP-Cy5.5 (Biolegend), TCRβ BV510 (Biolegend), CCR7 PECy7 (Biolegend), H-2Kb PE (Biolegend), CD69 BV421 (Biolegend), CD45R/B220 APCFire750 (Biolegend), CD25 APCFire750 (Biolegend), GL3 APCFire750 (Biolegend), and NK1.1 APCFire750 (Biolegend). Thymocytes were also stained intracellularly with anti-Nur77 APC (BD) using the eBioscience Foxp3/Transcription factor staining buffer set, as described earlier.

Tumor Digestion

B16 tumors from WT and Yap-cKO mice were dissected, mechanically disrupted, and digested in serum-free media containing 2 mg/ml collagenase type I (Worthington) and DNase I (Sigma) for 30 minutes at 37° C. in a rotator. Tumor digests were then passed through a 70 m mesh (Falcon) using an insulin syringe plunger and washed with PBS. Cells were treated with ACK red blood cell lysis buffer (Gibco), and tumor single-cell suspensions were prepared for staining and analysis by flow cytometry, using DAPI (Biolegend) and antibodies for CD45 BV510 (Biolegend), CD3 BUV737 (BD), CD4 BUV395 (BD), and CD8 PerCP-Cy5.5 (Biolegend). For determining absolute numbers of tumor-infiltrating T cells by flow cytometry, AccuCount fluorescent particles (Spherotech) were added to the tumor digests.

Adoptive Cell Transfers

For the adoptive cell transfers, EYFP+ Yap-cKO CD8⁺ T cells were mixed 1:1 with tdTomato⁺ WT CD8⁺ T cells and injected intravenously into 8-week-old WT C57BL/6 mice (Taconic) on day 0. Mice also received a subcutaneous injection of 5×10⁴ B16F10 cells on the same day. On day 15 of tumor growth, tumors were harvested and stained for flow cytometric analysis with dead cell dye, DAPI (Biolegend), and antibodies recognizing CD45 BV510 (Biolegend), CD3 BUV737 (BD), CD4 APC (Biolegend), and CD8 PerCP-Cy5.5 (Biolegend).

Immunofluorescence Microscopy

Harvested B16 tumors were fixed overnight in PLP fixative, followed by incubation in 15% and 30% sucrose. Tumors were embedded in OCT and frozen. Cryosections were cut at 5 μm thickness and stored at −20° C. Slides were stained with rat anti-mouse CD8 (clone CT-CD8a, Fisher) and donkey anti-rat Alexa 647 (Jackson Immuno Research Labs). Slides were washed and mounted in ProLong antifade reagent with DAPI (Life Technologies). Images were acquired using an AxioObserver D1 equipped with an X-Cite 120LED System.

Immunoblotting and Quantitative Real-Time PCR

RNA was extracted using Rneasy Mini Kit (Qiagen), and 1 g was used to generate cDNA using an iScript cDNA Synthesis Kit (Bio-rad). Taqman primers (Life Tech) for mouse Gapdh (4352339E), Yap (Mm01143263_ml), or TEAD1-4 (Mm00493507_m1, Mm00449004_m1, Mm00449013_m1, Mm01189836_m1) were mixed with cDNA and Taqman Universal Master Mix II (Life Tech), and ddC_{T} values were calculated relative to unstimulated controls. Protein lysates were analyzed by immunoblotting with anti-YAP (D8H1X) XP (CST 14074) and anti-GAPDH (D16H11) XP (CST 8884) antibodies and were imaged using a Bio-Rad ChemiDoc system.

Sample Preparation for RNA-Seq

B16 tumors from WT and Yap-cKO mice were digested as described earlier. Tumors cells were subsequently stained with DAPI and antibodies recognizing CD45 V500 (Biolegend), CD3 PE (Biolegend), CD4 APC (Biolegend), CD8 PerCP-Cy5.5 (Biolegend), and CD4⁺ and CD8⁺ TILs were sorted from each tumor using a BD FACSAria instrument. CD4⁺ and CD8⁺ T cells were sorted from WT and Yap-cKO TDLNs, as well. Cells were sorted into TRIzol LS reagent (Invitrogen), and RNA was isolated using a miRNeasy micro kit (Qiagen).

Transcriptomic Analyses and Gene Expression Signature Extraction

RNA quality was evaluated using Agilent Bioanalyzer 2100 Eukaryote Total RNA Pico chips. RNA-seq libraries were prepared using the SMART-Seq version 4 Ultra Low Input RNA Kit (Takara, 634889) from total RNA, following the manufacturer's protocol. Libraries were then sequenced on a HiSeq 4000 using 75-bp paired end reads to an average depth of 22,445,650 240,398 reads (SEM). Transcript abundance estimates were quantified using Salmon to mouse reference transcriptome from assembly GRCm38 (mm10), aggregated to gene level for UCSC-annotated genes using tximport, and DESeq2 was used to calculate normalized counts and differential expression [88, 89]. CD4 and CD8 up/down gene signatures were generated through differential expression analysis via DESeq2. Differentially expressed genes (DEGs) between Yap-cKO versus WT CD4⁺ and CD8⁺ cells were defined as log 2(FC)>1 (up) or log 2(FC)<−1 (down) and FDR<0.05. DEGs were visualized with a heatmap combined with a barplot annotation, with the heatmap cells representing the log-normalized expression values for each sample. Each row is accompanied by a bar representing the log-fold change in gene expression (KO/WT). Plots were generated using the ComplexHeatmap software package available in R. Data have been deposited in NCBI GEO (accession number GSE139883).

Analysis of Gene Expression Signatures in TCGA Datasets

The activation of CD4/8 up and down signatures was calculated with Gene Set Variation Analysis (GSVA) [90] in primary tumor samples across multiple TCGA RNA-seq datasets. Signature activation was summarized by the sum of activation of the up-regulated signature and inactivation of the down-regulated signature. For example, CD4_{sig} activity=GSVA(CD4_{up sig}) GSVA(CD4_{down sig}). Heatmaps for all TCGA analyses were generated with the pheatmap software package available in R. TCGA data include count matrices generated with STAR2/HTSeq downloaded from Genomic Data Commons (GDC) Data Portal. Each count matrix was normalized by relative log expression (RLE) with DESeq2. This analysis was constrained to samples for which survival information was available and immune infiltration could be estimated with Tumor Immune Estimation Resource (TIMER) [91]. The average T-cell infiltration per sample was estimated with TIMER in each TCGA dataset, represented as a single heatmap cell. This average was measured separately for CD4 T-cell and CD8 T-cell infiltration. Additionally, these values were summed to observe their additive effect. Transcription factor motif analysis was performed using the HOMER de novo motif analysis tool [92].

For correlation of signatures with T-cell infiltration, CD4/8 signature activation was correlated with the sum of CD4⁺ T-cell and CD8⁺ T-cell infiltration in each TCGA dataset estimated by TIMER [91]. The heatmap in FIG. 5I is colored by the correlation coefficient, and the text of each cell is the adjusted p-value of the correlation. For survival analysis, the CD4/8 signature activation was used to stratify patients across TCGA datasets into high or low activated groups separated by the mean. For each dataset, Kaplan-Meier survival plots were generated, and the results were summarized with the heatmap shown in FIG. 5J and FIG. 12B. Red signifies that an average survival probability is higher for patients with high activity of Yap-regulated signature, whereas dark blue signifies that an average survival probability is higher for patients with low Yap activity. Light orange and light blue signify the same groups with higher survival probability, respectively, but the differences are not significant. The text of each cell is the p-value for the survival estimation. The distribution ofp-values arising from the multiple survival analyses for each signature across TCGA datasets was compared to a uniform distribution using a Kolmogorov-Smirnov test.

Abbreviations: ACC, adrenocortical carcinoma; BLCA, bladder urothelial carcinoma; BRCA, breast invasive carcinoma; CAR-T, chimeric antigen receptor T cell; CD, cluster of differentiation; CESC, cervical squamous cell carcinoma and endocervical adenocarcinoma; CHOL, cholangiocarcinoma; cKO, conditional knockout; COAD, colon adenocarcinoma; DEG, differentially expressed gene; DLBC, lymphoid neoplasm diffuse large B-cell lymphoma; DLN, draining lymph node; DN, double negative; DP, double positive; dTom, dTomato; ESCA, esophageal carcinoma; EYFP, enhanced yellow fluorescent protein; Foxp3, forkhead box protein 3; GAPDH, glyceraldehyde 3-phosphate dehydrogenase; GATA3, GATA binding protein 3; GBM, glioblastoma multiforme; GDC, Genomic Data Commons; GEO, Gene Expression Omnibus; GSVA, Gene Set Variation Analysis; HNSC, head and neck squamous cell carcinoma; IFNγ, interferon gamma; IL-2, interleukin 2; IL-4, interleukin 4; Iono, ionomycin; JAK-STAT, Janus kinase-signal transducers and activators of transcription; KICH, kidney chromophobe; KIRC, kidney renal clear cell carcinoma; KIRP, kidney renal papillary cell carcinoma; k.s., Kolmogorov-Smirnov; LATS1, Large tumor suppressor kinase 1; LGG, brain lower grade glioma; LIHC, liver hepatocellular carcinoma; LLC, Lewis lung carcinoma; LoxP, locus of X-over P1; LUAD, lung adenocarcinoma; LUAD, lung adenocarcinoma; LUSC, lung squamous cell carcinoma; MAPK, mitogen-activated protein kinase; MESO, mesothelioma; MFI, median fluorescence intensity, MHC, major histocompatibility complex; MHCI, major histocompatibility complex class I; mTOR, mammalian target of rapamycin; MST1, mammalian sterile 20 like 1; MST2, mammalian sterile 20-like 2; NFAT, nuclear factor of activated T cells; NCBI, National Center for Biotechnology Information; NFkB, nuclear factor kappa-light-chain-enhancer of activated B cells; No Stim, no stimulation; Nur77, nuclear receptor 77; PMA, phorbol 12-myristate 13-acetate; OV, ovarian serous cystadenocarcinoma; PAAD, pancreatic adenocarcinoma; PCPG, pheochromocytoma and paraganglioma; PRAD, prostate adenocarcinoma; READ, rectum adenocarcinoma; RNA-seq, RNA sequencing; SARC, sarcoma; SKCM, skin cutaneous melanoma; STAD, stomach adenocarcinoma; TCGA, The Cancer Genome Atlas; TDLN, tumor-draining lymph node; TEAD, TEA domain family member; TGCT, testicular germ cell tumors; Th, T helper cell type; THCA, thyroid carcinoma; THYM, thymoma; TIL, tumor-infiltrating lymphocyte; Treg, Regulatory T cell; RLE, relative log expression; RORγt, RAR-related orphan receptor γt; SKCM, skin cutaneous melanoma; SM, semimature; SP, single positive; T-BET, T-box protein expressed in T cells; TCGA, The Cancer Genome Atlas; TCR, T cell receptor; TDLN, tumor-draining lymph node; TGFβ, transforming growth factor beta; Th1, T helper cell type 1; TIL, tumor-infiltrating lymphocyte; TIMER, Tumor Immune Estimation Resource; Treg, regulatory T cell; UCEC, uterine corpus endometrial carcinoma; UCS, uterine carcinosarcoma; UVM, uveal melanoma; WT, wild-type; Yap, Yes-associated protein

### REFERENCES

1. Kumar B V, Connors T J, Farber D L. Human T Cell Development, Localization, and Function throughout Life. Immunity. 2018; 48(2):202-13. Epub 2018/02/22. doi: 10.1016/j.immuni.2018.01.007. PubMed PMID: 29466753; PubMed Central PMCID: PMCPMC5826622.
2. Williams M A, Bevan M J. Effector and memory CTL differentiation. Annual review of immunology. 2007; 25:171-92. Epub 2006/11/30. doi: 10.1146/annurev.immunol.25.022106.141548. PubMed PMID: 17129182.
3. Luckheeram R V, Zhou R, Verma A D, Xia B. CD4(+)T cells: differentiation and functions. Clinical & developmental immunology. 2012; 2012:925135. Epub 2012/04/05. doi: 10.1155/2012/925135. PubMed PMID: 22474485; PubMed Central PMCID: PMCPMC3312336.
4. Murakami N, Riella L V. Co-inhibitory pathways and their importance in immune regulation. Transplantation. 2014; 98(1):3-14. Epub 2014/07/01. doi: 10.1097/tp.0000000000000169. PubMed PMID: 24978034.
5. Wherry E J, Kurachi M. Molecular and cellular insights into T cell exhaustion. Nat Rev Immunol. 2015; 15(8):486-99. Epub 2015/07/25. doi: 10.1038/nri3862. PubMed PMID: 26205583; PubMed Central PMCID: PMCPMC4889009.
6. Pauken K E, Wherry E J. Overcoming T cell exhaustion in infection and cancer. Trends Immunol. 2015; 36(4):265-76. Epub 2015/03/24. doi: 10.1016/j.it.2015.02.008. PubMed PMID: 25797516; PubMed Central PMCID: PMCPMC4393798.
7. Brentjens R J, Davila M L, Riviere I, Park J, Wang X, Cowell L G, et al. CD19-targeted T cells rapidly induce molecular remissions in adults with chemotherapy-refractory acute lymphoblastic leukemia. Science translational medicine. 2013; 5(177):177ra38. Epub 2013/03/22. doi: 10.1126/scitranslmed.3005930. PubMed PMID: 23515080; PubMed Central PMCID: PMCPMC3742551.
8. Lee D W, Kochenderfer J N, Stetler-Stevenson M, Cui Y K, Delbrook C, Feldman S A, et al. T cells expressing CD19 chimeric antigen receptors for acute lymphoblastic leukaemia in children and young adults: a phase 1 dose-escalation trial. Lancet (London, England). 2015; 385(9967):517-28. Epub 2014/10/17. doi: 10.1016/s0140-6736(14)61403-3. PubMed PMID: 25319501.
9. Maude S L, Frey N, Shaw P A, Aplenc R, Barrett D M, Bunin N J, et al. Chimeric antigen receptor T cells for sustained remissions in leukemia. The New England journal of medicine. 2014; 371(16):1507-17. Epub 2014/10/16. doi: 10.1056/NEJMoa1407222. PubMed PMID: 25317870; PubMed Central PMCID: PMCPMC4267531.
10. Topalian S L, Hodi F S, Brahmer J R, Gettinger S N, Smith D C, McDermott D F, et al. Safety, activity, and immune correlates of anti-PD-1 antibody in cancer. The New England journal of medicine. 2012; 366(26):2443-54. Epub 2012/06/05. doi: 10.1056/NEJMoa200690. PubMed PMID: 22658127; PubMed Central PMCID: PMCPMC3544539.
11. Zhou H, Luo Y, Zhu S, Wang X, Zhao Y, Ou X, et al. The efficacy and safety of anti-CD19/CD20 chimeric antigen receptor-T cells immunotherapy in relapsed or refractory B-cell malignancies:a meta-analysis. BMC cancer. 2018; 18(1):929. Epub 2018/09/28. doi: 10.1186/s12885-018-4817-4. PubMed PMID: 30257649; PubMed Central PMCID: PMCPMC6158876.
12. Feng Y, Roy A, Masson E, Chen T T, Humphrey R, Weber J S. Exposure-response relationships of the efficacy and safety of ipilimumab in patients with advanced melanoma. Clinical cancer research: an official journal of the American Association for Cancer Research. 2013; 19(14):3977-86. Epub 2013/06/07. doi: 10.1158/1078-0432.Ccr-12-3243. PubMed PMID: 23741070.
13. Rizvi N A, Hellmann M D, Snyder A, Kvistborg P, Makarov V, Havel J J, et al. Cancer immunology. Mutational landscape determines sensitivity to PD-1 blockade in non-small cell lung cancer. Science (New York, N.Y.). 2015; 348(6230):124-8. Epub 2015/03/15. doi: 10.1126/science.aaa348. PubMed PMID: 25765070; PubMed Central PMCID: PMCPMC4993154.
14. Herbst R S, Soria J C, Kowanetz M, Fine G D, Hamid O, Gordon M S, et al. Predictive correlates of response to the anti-PD-L1 antibody MPDL3280A in cancer patients. Nature. 2014; 515(7528):563-7. Epub 2014/11/28. doi: 10.1038/nature14011. PubMed PMID: 25428504; PubMed Central PMCID: PMCPMC4836193.
15. Galon J, Pages F, Marincola F M, Angell H K, Thurin M, Lugli A, et al. Cancer classification using the Immunoscore: a worldwide task force. Journal of translational medicine. 2012; 10:205. Epub 2012/10/05. doi: 10.1186/1479-5876-10-205. PubMed PMID: 23034130; PubMed Central PMCID: PMCPMC3554496.
16. Pages F, Berger A, Camus M, Sanchez-Cabo F, Costes A, Molidor R, et al. Effector memory T cells, early metastasis, and survival in colorectal cancer. The New England journal of medicine. 2005; 353(25):2654-66. Epub 2005/12/24. doi: 10.1056/NEJMoa051424. PubMed PMID: 16371631.
17. Galon J, Costes A, Sanchez-Cabo F, Kirilovsky A, Mlecnik B, Lagorce-Pages C, et al. Type, density, and location of immune cells within human colorectal tumors predict clinical outcome. Science (New York, N.Y.). 2006; 313(5795):1960-4. Epub 2006/09/30. doi: 10.1126/science.1129139. PubMed PMID: 17008531.
18. Mlecnik B, Tosolini M, Kirilovsky A, Berger A, Bindea G, Meatchi T, et al. Histopathologic-based prognostic factors of colorectal cancers are associated with the state of the local immune reaction. Journal of clinical oncology: official journal of the American Society of Clinical Oncology. 2011; 29(6):610-8. Epub 2011/01/20. doi: 10.1200/jco.2010.30.5425. PubMed PMID: 21245428.
19. Hamid O, Schmidt H, Nissan A, Ridolfi L, Aamdal S, Hansson J, et al. A prospective phase II trial exploring the association between tumor microenvironment biomarkers and clinical activity of ipilimumab in advanced melanoma. Journal of translational medicine. 2011; 9:204. Epub 2011/11/30. doi: 10.1186/1479-5876-9-204. PubMed PMID: 22123319; PubMed Central PMCID: PMCPMC3239318.
20. Darvin P, Toor S M, Sasidharan Nair V, Elkord E. Immune checkpoint inhibitors: recent progress and potential biomarkers. Experimental & molecular medicine. 2018; 50(12):165. Epub 2018/12/14. doi: 10.1038/s12276-018-0191-1. PubMed PMID: 30546008; PubMed Central PMCID: PMCPMC6292890.
21. Brown C E, Mackall C L. CAR T cell therapy: inroads to response and resistance. Nat Rev Immunol. 2019; 19(2):73-4. Epub 2019/01/12. doi: 10.1038/s41577-018-0119-y. PubMed PMID: 30631206.
22. Cogdill A P, Andrews M C, Wargo J A. Hallmarks of response to immune checkpoint blockade. British journal of cancer. 2017; 117(1):1-7. Epub 2017/05/20. doi: 10.1038/bjc.2017.136. PubMed PMID: 28524159; PubMed Central PMCID: PMCPMC5520201.
23. Balatoni T, Mohos A, Papp E, Sebestyen T, Liszkay G, Olah J, et al. Tumor-infiltrating immune cells as potential biomarkers predicting response to treatment and survival in patients with metastatic melanoma receiving ipilimumab therapy. Cancer immunology, immunotherapy: CII. 2018; 67(1):141-51. Epub 2017 Dec. 11. doi: 10.1007/s00262-017-2072-1. PubMed PMID: 28988380.
24. Gajewski T F, Woo S R, Zha Y, Spaapen R, Zheng Y, Corrales L, et al. Cancer immunotherapy strategies based on overcoming barriers within the tumor microenvironment. Current opinion in immunology. 2013; 25(2):268-76. Epub 2013/04/13. doi: 10.1016/j.coi.2013.02.009. PubMed PMID: 23579075.
25. Varelas X. The Hippo pathway effectors TAZ and YAP in development, homeostasis and disease. Development. 2014; 141(8):1614-26. doi: 10.1242/dev.102376. PubMed PMID: 24715453.
26. Piccolo S, Dupont S, Cordenonsi M. The biology of YAP/TAZ: hippo signaling and beyond. Physiological reviews. 2014; 94(4):1287-312. doi: 10.1152/physrev.00005.2014. PubMed PMID: 25287865.
27. Thaventhiran J E, Hoffmann A, Magiera L, de la Roche M, Lingel H, Brunner-Weinzierl M, et al. Activation of the Hippo pathway by CTLA-4 regulates the expression of Blimp-1 in the CD8⁺ T cell. Proc Natl Acad Sci USA. 2012; 109(33):E2223-9. Epub 2012/06/30. doi: 10.1073/pnas.1209115109. PubMed PMID: 22745171; PubMed Central PMCID: PMCPMC3421161.
28. Dong Y, Du X, Ye J, Han M, Xu T, Zhuang Y, et al. A cell-intrinsic role for Mst1 in regulating thymocyte egress. Journal of immunology (Baltimore, Md.: 1950). 2009; 183(6):3865-72. Epub 2009/08/21. doi: 10.4049/jimmunol.0900678. PubMed PMID: 19692642.
29. Katagiri K, Imamura M, Kinashi T. Spatiotemporal regulation of the kinase Mst by binding protein RAPL is critical for lymphocyte polarity and adhesion. Nature immunology. 2006; 7(9):919-28. Epub 2006/08/08. doi: 10.1038/ni374. PubMed PMID: 16892067.
30. Katagiri K, Katakai T, Ebisuno Y, Ueda Y, Okada T, Kinashi T. Mst1 controls lymphocyte trafficking and interstitial motility within lymph nodes. The EMBO journal. 2009; 28(9):1319-31. Epub 2009/04/03. doi: 10.1038/emboj.2009.82. PubMed PMID: 19339990; PubMed Central PMCID: PMCPMC2683056.
31. Mou F, Praskova M, Xia F, Van Buren D, Hock H, Avruch J, et al. The Mst1 and Mst2 kinases control activation of rho family GTPases and thymic egress of mature thymocytes. The Journal of experimental medicine. 2012; 209(4):741-59. Epub 2012/03/14. doi: 10.1084/jem.20111692. PubMed PMID: 22412158; PubMed Central PMCID: PMCPMC3328371.
32. Nehme N T, Schmid J P, Debeurme F, Andre-Schmutz I, Lim A, Nitschke P, et al. MST mutations in autosomal recessive primary immunodeficiency characterized by defective naïve T-cell survival. Blood. 2012; 119(15):3458-68. Epub 2011/12/17. doi: 10.1182/blood-2011-09-378364. PubMed PMID: 22174160; PubMed Central PMCID: PMCPMC3824282.
33. Nishikimi A, Ishihara S, Ozawa M, Etoh K, Fukuda M, Kinashi T, et al. Rab13 acts downstream of the kinase Mst1 to deliver the integrin LFA-1 to the cell surface for lymphocyte trafficking. Science signaling. 2014; 7(336):ra72. Epub 2014/07/31. doi: 10.1126/scisignal.2005199. PubMed PMID: 25074980.
34. Tang F, Gill J, Ficht X, Barthlott T, Cornils H, Schmitz-Rohmer D, et al. The kinases NDR1/2 act downstream of the Hippo homolog MST1 to mediate both egress of thymocytes from the thymus and lymphocyte motility. Science signaling. 2015; 8(397):ra100. Epub 2015/10/08. doi: 10.1126/scisignal.aab2425. PubMed PMID: 26443704.
35. Ueda Y, Katagiri K, Tomiyama T, Yasuda K, Habiro K, Katakai T, et al. Mst regulates integrin-dependent thymocyte trafficking and antigen recognition in the thymus. Nature communications. 2012; 3:1098. Epub 2012/10/04. doi: 10.1038/ncomms2105. PubMed PMID: 23033074.
36. Zhou D, Medoff B D, Chen L, Li L, Zhang X F, Praskova M, et al. The NoreB/Mst1 complex restrains antigen receptor-induced proliferation of naïve T cells. Proc Natl Acad Sci USA. 2008; 105(51):20321-6. Epub 2008/12/17. doi: 10.1073/pnas.0810773105. PubMed PMID: 19073936; PubMed Central PMCID: PMCPMC2600581.
37. Ni X, Tao J, Barbi J, Chen Q, Park B V, Li Z, et al. YAP Is Essential for Treg-Mediated Suppression of Antitumor Immunity. Cancer Discov. 2018; 8(8):1026-43. Epub 2018/06/17. doi: 10.1158/2159-8290.CD-17-1124. PubMed PMID: 29907586; PubMed Central PMCID: PMCPMC6481611.
38. Miyake K, Underhill C B, Lesley J, Kincade P W. Hyaluronate can function as a cell adhesion molecule and CD44 participates in hyaluronate recognition. The Journal of experimental medicine. 1990; 172(1):69-75. Epub 1990/07/01. doi: 10.1084/jem.172.1.69. PubMed PMID: 2193100; PubMed Central PMCID: PMCPMC2188161.
39. Budd R C, Cerottini J C, Horvath C, Bron C, Pedrazzini T, Howe R C, et al. Distinction of virgin and memory T lymphocytes. Stable acquisition of the Pgp-1 glycoprotein concomitant with antigenic stimulation. J Immunol. 1987; 138(10):3120-9. Epub 1987/05/15. PubMed PMID: 3106474.
40. Ziegler S F, Ramsdell F, Alderson M R. The activation antigen CD69. Stem Cells. 1994; 12(5):456-65. Epub 1994/09/01. doi: 10.1002/stem.5530120502. PubMed PMID: 7804122.
41. Malek T R, Robb R J, Shevach E M. Identification and initial characterization of a rat monoclonal antibody reactive with the murine interleukin 2 receptor-ligand complex. Proc Natl Acad Sci USA. 1983; 80(18):5694-8. Epub 1983/09/01. doi: 10.1073/pnas.80.18.5694. PubMed PMID: 6412230; PubMed Central PMCID: PMCPMC384325.
42. Liu-Chittenden Y, Huang B, Shim J S, Chen Q, Lee S J, Anders R A, et al. Genetic and pharmacological disruption of the TEAD-YAP complex suppresses the oncogenic activity of YAP. Genes &development. 2012; 26(12):1300-5. Epub 2012/06/09. doi: 10.1101/gad.192856.112. PubMed PMID: 22677547; PubMed Central PMCID: PMCPMC3387657.
43. Krebs C F, Turner J E, Paust H J, Kapffer S, Koyro T, Krohn S, et al. Plasticity of Th17 Cells in Autoimmune Kidney Diseases. Journal of immunology (Baltimore, Md.: 1950). 2016; 197(2):449-57. Epub 2016/06/09. doi: 10.4049/jimmunol.1501831. PubMed PMID: 27271566.
44. Geng J, Yu S, Zhao H, Sun X, Li X, Wang P, et al. The transcriptional coactivator TAZ regulates reciprocal differentiation of TH17 cells and Treg cells. Nature immunology. 2017; 18(7):800-12. Epub 2017/05/16. doi: 10.1038/ni.3748. PubMed PMID: 28504697.
45. Takaba H, Takayanagi H. The Mechanisms of T Cell Selection in the Thymus. Trends Immunol. 2017; 38(11):805-16. Epub 2017/08/24. doi: 10.1016/j.it.2017.07.010. PubMed PMID: 28830733.
46. Xing Y, Wang X, Jameson S C, Hogquist K A. Late stages of T cell maturation in the thymus involve NF-kappaB and tonic type I interferon signaling. Nat Immunol. 2016; 17(5):565-73. Epub 2016/04/05. doi: 10.1038/ni.3419. PubMed PMID: 27043411; PubMed Central PMCID: PMCPMC4837029.
47. Ashouri J F, Weiss A. Endogenous Nur77 Is a Specific Indicator of Antigen Receptor Signaling in Human T and B Cells. J Immunol. 2017; 198(2):657-68. Epub 2016/12/13. doi: 10.4049/jimmunol.1601301. PubMed PMID: 27940659; PubMed Central PMCID: PMCPMC5224971.
48. Santis A G, Lopez-Cabrera M, Sanchez-Madrid F, Proudfoot N. Expression ofthe early lymphocyte activation antigen CD69, a C-type lectin, is regulated by mRNA degradation associated with AU-rich sequence motifs. Eur J Immunol. 1995; 25(8):2142-6. Epub 1995/08/01. doi: 10.1002/eji.1830250804. PubMed PMID: 7664776.
49. Feng C, Woodside K J, Vance B A, El-Khoury D, Canelles M, Lee J, et al. A potential role for CD69 in thymocyte emigration. Int Immunol. 2002; 14(6):535-44. Epub 2002/06/01. doi: 10.1093/intimm/dxfO20. PubMed PMID: 12039905.
50. Jung L K, Haynes B F, Nakamura S, Pahwa S, Fu S M. Expression of early activation antigen (CD69) during human thymic development. Clin Exp Immunol. 1990; 81(3):466-74. Epub 1990/09/01. doi: 10.1111/j.1365-2249.1990.tb05357.x. PubMed PMID: 2204504; PubMed Central PMCID: PMCPMC1534971.
51. Hogquist K A, Xing Y, Hsu F C, Shapiro V S. T Cell Adolescence: Maturation Events Beyond Positive Selection. J Immunol. 2015; 195(4):1351-7. Epub 2015/08/09. doi: 10.4049/jimmunol.1501050. PubMed PMID: 26254267; PubMed Central PMCID: PMCPMC4530466.
52. Liu Z G, Smith S W, McLaughlin K A, Schwartz L M, Osborne B A. Apoptotic signals delivered through the T-cell receptor of a T-cell hybrid require the immediate-early gene nur77. Nature. 1994; 367(6460):281-4. Epub 1994/01/20. doi: 10.1038/367281a0. PubMed PMID: 8121494.
53. Calnan B J, Szychowski S, Chan F K, Cado D, Winoto A. A role for the orphan steroid receptor Nur77 in apoptosis accompanying antigen-induced negative selection. Immunity. 1995; 3(3):273-82. Epub 1995/09/01. doi: 10.1016/1074-7613(95)90113-2. PubMed PMID: 7552993.
54. Celik C, Lewis D A, Goldrosen M H. Demonstration of immunogenicity with the poorly immunogenic B16 melanoma. Cancer research. 1983; 43(8):3507-10. Epub 1983/08/01. PubMed PMID: 6861123.
55. Turk M J, Guevara-Patino J A, Rizzuto G A, Engelhorn M E, Sakaguchi S, Houghton A N. Concomitant tumor immunity to a poorly immunogenic melanoma is prevented by regulatory T cells. The Journal of experimental medicine. 2004; 200(6):771-82. Epub 2004/09/24. doi: 10.1084/jem.20041130. PubMed PMID: 15381730; PubMed Central PMCID: PMCPMC2211964.
56. Geiger J D, Wagner P D, Cameron M J, Shu S, Chang A E. Generation of T-cells reactive to the poorly immunogenic B16-BL6 melanoma with efficacy in the treatment of spontaneous metastases. Journal of immunotherapy with emphasis on tumor immunology: official journal of the Society for Biological Therapy. 1993; 13(3):153-65. Epub 1993/04/01. PubMed PMID: 8471590.
57. Wang J, Saffold S, Cao X, Krauss J, Chen W. Eliciting T cell immunity against poorly immunogenic tumors by immunization with dendritic cell-tumor fusion vaccines. Journal of immunology (Baltimore, Md.: 1950). 1998; 161(10):5516-24. Epub 1998/11/20. PubMed PMID: 9820528.
58. Galon J, Angell H K, Bedognetti D, Marincola F M. The continuum of cancer immunosurveillance: prognostic, predictive, and mechanistic signatures. Immunity. 2013; 39(1):11-26. Epub 2013/07/31. doi: 10.1016/j.immuni.2013.07.008. PubMed PMID: 23890060.
59. Mlecnik B, Bindea G, Angell H K, Maby P, Angelova M, Tougeron D, et al. Integrative Analyses of Colorectal Cancer Show Immunoscore Is a Stronger Predictor of Patient Survival Than Microsatellite Instability. Immunity. 2016; 44(3):698-711. Epub 2016/03/18. doi: 10.1016/j.immuni.2016.02.025. PubMed PMID: 26982367.
60. Galluzzi L, Chan T A, Kroemer G, Wolchok J D, Lopez-Soto A. The hallmarks of successful anticancer immunotherapy. Science translational medicine. 2018; 10(459). Epub 2018/09/21. doi: 10.1126/scitranslmed.aat7807. PubMed PMID: 30232229.
61. Gajewski T F. Failure at the effector phase: immune barriers at the level of the melanoma tumor microenvironment. Clinical cancer research: an official journal ofthe American Association for Cancer Research. 2007; 13(18 Pt 1):5256-61. Epub 2007/09/19. doi: 10.1158/1078-0432.Ccr-07-0892. PubMed PMID: 17875753.
62. Pages F, Mlecnik B, Marliot F, Bindea G, Ou F S, Bifulco C, et al. International validation of the consensus Immunoscore for the classification of colon cancer: a prognostic and accuracy study. Lancet (London, England). 2018; 391(10135):2128-39. Epub 2018/05/15. doi: 10.1016/s0140-6736(18)30789-x. PubMed PMID: 29754777.
63. Federico A, Monti S. hypeR: Geneset enrichment analysis based on hyper-geometric test. Bioconductor. 2019. doi: doi:10.18129/B9.bioc.hypeR.
64. Stubbington M J, Mahata B, Svensson V, Deonarine A, Nissen J K, Betz A G, et al. An atlas of mouse CD4(+) T cell transcriptomes. Biology direct. 2015; 10:14. Epub 2015/04/19. doi: 10.1186/s13062-015-0045-x. PubMed PMID: 25886751; PubMed Central PMCID: PMCPMC4384382.
65. Kobayashi M, Kobayashi H, Pollard R B, Suzuki F. A pathogenic role of Th2 cells and their cytokine products on the pulmonary metastasis of murine B16 melanoma. J Immunol. 1998; 160(12):5869-73. Epub 1998/06/24. PubMed PMID: 9637498.
66. Curran M A, Montalvo W, Yagita H, Allison J P. PD-1 and CTLA-4 combination blockade expands infiltrating T cells and reduces regulatory T and myeloid cells within B16 melanoma tumors. Proc Natl Acad Sci USA. 2010; 107(9):4275-80. Epub 2010/02/18. doi: 10.1073/pnas.0915174107. PubMed PMID: 20160101; PubMed Central PMCID: PMCPMC2840093.
67. Nakagawa H, Sido J M, Reyes E E, Kiers V, Cantor H, Kim H J. Instability of Helios-deficient Tregs is associated with conversion to a T-effector phenotype and enhanced antitumor immunity. Proc Natl Acad Sci USA. 2016; 113(22):6248-53. Epub 2016/05/18. doi: 10.1073/pnas.1604765113. PubMed PMID: 27185917; PubMed Central PMCID: PMCPMC4896716.
68. Weinstein J N, Collisson E A, Mills G B, Shaw K R, Ozenberger B A, Ellrott K, et al. The Cancer Genome Atlas Pan-Cancer analysis project. Nature genetics. 2013; 45(10):1113-20. Epub 2013/09/28. doi: 10.1038/ng.2764. PubMed PMID: 24071849; PubMed Central PMCID: PMCPMC3919969.
69. Smith-Garvin J E, Koretzky G A, Jordan M S. T cell activation. Annual review of immunology. 2009; 27:591-619. Epub 2009/01/10. doi: 10.1146/annurev.immunol.021908.132706. PubMed PMID: 19132916; PubMed Central PMCID: PMCPMC2740335.
70. Bretscher P A. A two-step, two-signal model for the primary activation of precursor helper T cells. Proc Natl Acad Sci USA. 1999; 96(1):185-90. Epub 1999/01/06. PubMed PMID: 9874793; PubMed Central PMCID: PMCPMC15114.
71. Curtsinger J M, Schmidt C S, Mondino A, Lins D C, Kedl R M, Jenkins M K, et al. Inflammatory cytokines provide a third signal for activation of naïve CD4+ and CD8⁺ T cells. Journal of immunology (Baltimore, Md.: 1950). 1999; 162(6):3256-62. Epub 1999/03/27. PubMed PMID: 10092777.
72. Cunningham N R, Artim S C, Fornadel C M, Sellars M C, Edmonson S G, Scott G, et al. Immature CD4+CD8+ thymocytes and mature T cells regulate Nur77 distinctly in response to TCR stimulation. J Immunol. 2006; 177(10):6660-6. Epub 2006 Nov. 4. doi: 10.4049/jimmunol.177.10.6660. PubMed PMID: 17082578.
73. Liu-Chittenden Y, Huang B, Shim J S, Chen Q, Lee S J, Anders R A, et al. Genetic and pharmacological disruption of the TEAD-YAP complex suppresses the oncogenic activity of YAP. Genes Dev. 2012; 26(12):1300-5. Epub 2012/06/09. doi: 10.1101/gad.192856.112. PubMed PMID: 22677547; PubMed Central PMCID: PMC3387657.
74. Au-Yeung B B, Zikherman J, Mueller J L, Ashouri J F, Matloubian M, Cheng D A, et al. A sharp T-cell antigen receptor signaling threshold for T-cell proliferation. Proc Natl Acad Sci USA. 2014; 111(35):E3679-88. Epub 2014/08/20. doi: 10.1073/pnas.1413726111. PubMed PMID: 25136127; PubMed Central PMCID: PMCPMC4156735.
75. Allison K A, Sajti E, Collier J G, Gosselin D, Troutman T D, Stone E L, et al. Affinity and dose of TCR engagement yield proportional enhancer and gene activity in CD4⁺ T cells. Elife. 2016; 5. Epub 2016/07/05. doi: 10.7554/eLife.10134. PubMed PMID: 27376549; PubMed Central PMCID: PMCPMC4931909.
76. Zheng Y, Collins S L, Lutz M A, Allen A N, Kole T P, Zarek P E, et al. A role for mammalian target of rapamycin in regulating T cell activation versus anergy. J Immunol. 2007; 178(4):2163-70. Epub 2007/02/06. PubMed PMID: 17277121.
77. Chae W J, Bothwell A L M. Canonical and Non-Canonical Wnt Signaling in Immune Cells. Trends Immunol. 2018; 39(10):830-47. Epub 2018/09/15. doi: 10.1016/j.it.2018.08.006. PubMed PMID: 30213499.
78. Batlle E, Massague J. Transforming Growth Factor-beta Signaling in Immunity and Cancer. Immunity. 2019; 50(4):924-40. Epub 2019/04/18. doi: 10.1016/j.immuni.2019.03.024. PubMed PMID: 30995507.
79. Varelas X, Wrana J L. Coordinating developmental signaling: novel roles for the Hippo pathway. Trends Cell Biol. 2012; 22(2):88-96. Epub 2011/12/14. doi: 10.1016/j.tcb.2011.10.002. PubMed PMID: 22153608.
80. Chen D S, Mellman I. Elements of cancer immunity and the cancer-immune set point. Nature. 2017; 541(7637):321-30. Epub 2017/01/20. doi: 10.1038/nature21349. PubMed PMID: 28102259.
81. Woroniecka K, Chongsathidkiet P, Rhodin K, Kemeny H, Dechant C, Farber S H, et al. T-Cell Exhaustion Signatures Vary with Tumor Type and Are Severe in Glioblastoma. Clinical cancer research: an official journal of the American Association for Cancer Research. 2018; 24(17):4175-86. Epub 2018/02/14. doi: 10.1158/1078-0432.Ccr-17-1846. PubMed PMID: 29437767; PubMed Central PMCID: PMCPMC6081269.
82. Lechner M G, Karimi S S, Barry-Holson K, Angell T E, Murphy K A, Church C H, et al. Immunogenicity of murine solid tumor models as a defining feature of in vivo behavior and response to immunotherapy. Journal of immunotherapy (Hagerstown, Md.: 1997). 2013; 36(9):477-89. Epub 2013/10/23. doi: 10.1097/01.cji.0000436722.46675.4a. PubMed PMID: 24145359; PubMed Central PMCID: PMCPMC3910494.
83. Gajewski T F. The Next Hurdle in Cancer Immunotherapy: Overcoming the Non-T-Cell-Inflamed Tumor Microenvironment. Seminars in oncology. 2015; 42(4):663-71. Epub 2015/09/01. doi: 10.1053/j.seminoncol.2015.05.011. PubMed PMID: 26320069; PubMed Central PMCID: PMCPMC4555998.
84. Reginensi A, Scott R P, Gregorieff A, Bagherie-Lachidan M, Chung C, Lim D S, et al. Yap- and Cdc42-dependent nephrogenesis and morphogenesis during mouse kidney development. PLoS Genet. 2013; 9(3):e1003380. Epub 2013/04/05. doi: 10.1371/journal.pgen.1003380. PubMed PMID: 23555292; PubMed Central PMCID: PMC3605093.
85. Lee P P, Fitzpatrick D R, Beard C, Jessup H K, Lehar S, Makar K W, et al. A critical role for Dnmtl and DNA methylation in T cell development, function, and survival. Immunity. 2001; 15(5):763-74. Epub 2001/12/01. PubMed PMID: 11728338.
86. Srinivas S, Watanabe T, Lin C S, William C M, Tanabe Y, Jessell T M, et al. Cre reporter strains produced by targeted insertion of EYFP and ECFP into the ROSA26 locus. BMC Dev Biol. 2001; 1:4. Epub 2001/04/12. PubMed PMID: 11299042; PubMed Central PMCID: PMCPMC31338.
87. Madisen L, Zwingman T A, Sunkin S M, Oh S W, Zariwala H A, Gu H, et al. A robust and high-throughput Cre reporting and characterization system for the whole mouse brain. Nat Neurosci. 2010; 13(1):133-40. Epub 2009/12/22. doi: 10.1038/nn.2467. PubMed PMID: 20023653; PubMed Central PMCID: PMCPMC2840225.
88. Patro R, Duggal G, Love M I, Irizarry R A, Kingsford C. Salmon provides fast and bias-aware quantification of transcript expression. Nature methods. 2017; 14(4):417-9. Epub 2017/03/07. doi: 10.1038/nmeth.4197. PubMed PMID: 28263959; PubMed Central PMCID: PMCPMC5600148.
89. Love M I, Huber W, Anders S. Moderated estimation of fold change and dispersion for RNA-seq data with DESeq2. Genome Biol. 2014; 15(12):550. Epub 2014/12/18. doi: 10.1186/s13059-014-0550-8. PubMed PMID: 25516281; PubMed Central PMCID: PMCPMC4302049.
90. Hanzelmann S, Castelo R, Guinney J. GSVA: gene set variation analysis for microarray and RNA-seq data. BMC Bioinformatics. 2013; 14:7. Epub 2013/01/18. doi: 10.1186/1471-2105-14-7. PubMed PMID: 23323831; PubMed Central PMCID: PMCPMC3618321.
91. Li B, Severson E, Pignon J C, Zhao H, Li T, Novak J, et al. Comprehensive analyses of tumor immunity: implications for cancer immunotherapy. Genome Biol. 2016; 17(1):174. Epub 2016/08/24. doi: 10.1186/s13059-016-1028-7. PubMed PMID: 27549193; PubMed Central PMCID: PMCPMC4993001.
92. Heinz S, Benner C, Spann N, Bertolino E, Lin Y C, Laslo P, et al. Simple combinations of lineage-determining transcription factors prime cis-regulatory elements required for macrophage and B cell identities. Mol Cell. 2010; 38(4):576-89. Epub 2010/06/02. doi: 10.1016/j.molcel.2010.05.004. PubMed PMID: 20513432; PubMed Central PMCID: PMCPMC2898526.

### Example 2: Yap-Deleted CD8+ T Cells Exhibit Enhanced B16 Tumor Cell Killing Activity In Vitro

Described herein in this example are the results of experiments assessing the B16 tumor cell killing activity in vitro.

Yap-loxP/loxP; CD4-Cre; Thy1a; Tcra-V1/Tcrb-V13 (Yap-cko-pmel) C57BL/6 mice were generated, resulting in deletion of the Yap gene in CD4+ and CD8+ T cells of a mouse that carries a transgenic TCR that recognizes an epitope found on the poorly immunogenic B16 melanoma cell line. CD8⁺ T-cells were purified from splenocytes of control pmel mice (CD4-Cre; Thy1a; Tcra-V1/Tcrb-V13) and Yap-cko-pmel mice and then activated using anti-CD3/anti-CD28 coated Dynabeads at a 1:1 ratio for 48 hours. These CD8+ T cells were then mixed and co-cultured with B16 melanoma cells expressing Nano-Luciferase (B16-NanoLuc) growing in either 2D or 3D culture conditions. For 2D culture condition, B16-NanoLuc cells were seeded in 96-well plate overnight at a density of 5×10³ cells per well. On day 0, T cells were cocultured with B16-NanoLuc cells at 1:1 ratio. B16 cell number was quantified 48 hours after co-culturing with T cells. For 3D culture condition, 96-well plate was coated with 50 ul 1.5% agar per well. 5×10³ B16 cells were cultured for 72 hours, which allowed the cells to form spheroids. These spheroids were then co-cultured with T cells at 1:1 ratio and B16-NanoLuc cell number was quantified after 24 hours of co-culture. The growth of B16 was quantified using a Nano-Glo® Luciferase Assay System (Promega). Yap deleted CD8+ T cells show a significant decrease in B16 melanoma cells compared to wildtype T cells cultured as depicted in FIG. 18A and FIG. 18B. The results of these experiments show that Yap deleted CD8+ T cells exhibit enhanced B16 tumor cell killing activity in vitro.

### Example 3: Yap-Deleted T Cells Exhibit Higher Activation, Expansion, Markers of Tissue Homing Memory, and Th1-Skewed Cytokines when Grown on Different Stiffness Hydrogels In Vitro

Described herein in this example are the results of experiments assessing the activation, expansion and marker analyses of T-cells gown on different stiffness hydrogels in vitro.

Mouse CD8+ T cells were purified from splenocytes of C57BL/6J (WT) or CD4-Cre YAP-floxed C57BL/6J (YAP-cKO) mice. Cells were cultured either on hydrogels or uncoated 96-well tissue culture plates. Hydrogels were tested at low (0.5 kPa) and medium (1 kPa) stiffnesses. Hyaluronic acid-based hydrogels incorporated covalently conjugated anti-CD3, anti-CD28, and IL-2-Ig on the surface. Cells cultured on uncoated culture plates were incubated 1:1 with anti-CD3/anti-CD28-coated Dynabeads. Cells were activated for 7 days and analyzed for cell expansion compared to seeding density at day 0. Cell activation/tissue homing memory was determined by sustained CD69 levels, stained and analyzed by flow cytometry. Results show that YAP-cKO CD8+ T cells (FIG. 19A) expanded more on hydrogels, (FIG. 19B) retained higher CD69 levels after proliferation, and (FIG. 19C) produced more Th1 cytokines and less Th2 cytokines compared to expanded WT CD8+ T cells in both bead and HA hydrogel cultures.

### Example 4: The Yap Paralog Taz (Wwtr1) May be a Synergistic Immunotherapy Target with Yap Following Acute Yap-Inhibition

Described herein in this example are the results of experiments assessing the expression of the Yap paralog Taz (Wwtr1) following acute Yap inhibition.

Pan (CD4+ and CD8+) T cells were isolated from splenocytes of healthy C57BL/6J mice. Scrambled siRNA or YAP1 siRNA were delivered to freshly isolated T cells by nucleofection (Amaxa). Nucleofected T cells were plated and activated overnight with 1:1 anti-CD3/anti-CD28−-coated Dynabeads. Cells were collected at 24 h post-activation and RNA purified. Yap1 and Wwtr1 expression were determined using real-time RT-PCR Taqman probes and normalized to internal Actb control probes. Knockdown of YAP and upregulation of TAZ in activated T cells with YAP siRNA was calculated by normalization to activated T cells with scrambled siRNA (FIG. 20). Results show that siRNA-mediated depletion of Yap in T cells grown in vitro results in the increased expression of the Yap paralog Taz (Wwtr1) which can play redundant roles with Yap in several contexts (1). In previous studies in CD4+ T cells, Taz has been shown to be required for differentiating Th17 cells (2). This shows that the suppression of Yap may increase Th17 phenotypes through Taz upregulation and can be shifted by co-inhibition of Yap/Taz (FIG. 20). The results show that Taz (wwtr1) may be a synergistic immunotherapy target with Yap following acute Yap-inhibition.

### References (Example 4)

1) Geng et al. Nat Immunol. 2017 July; 18(7):800-812. doi: 10.1038/ni.3748. Epub 2017 May 15.
2) Varelas, Development. 2014 April; 141(8):1614-26. doi: 10.1242/dev.102376.

TABLE 3
Genes with altered expression in T cells with YAP inhibition

CD4, CSF1, CSF2, IL10, IL13, IL5, IL7, TNF, CD4, CSF1, CSF2, IL5, IL7, DUSP1, NFKB1, NFKBIA, TNFRSF1B,
TRAF1, JAK2, NFKB1, NFKBIA, TNF, TNFRSF1B, DUSP1, NFKB1, NFKBIA, NR3C1, TNF, NFKB1, NFKBIA, TNF,
TNFRSF1B, BIRC2, NFKB1, NFKBIA, TNF, TNFRSF1B, TRAF1, CSF2, IL10, IL13, IL5, CCR3, CD4, IL5, IL10, IL13, IL5,
TNF, NFKB1, NFKBIA, TNF, TNFRSF1B, JAK2, NFKB1, NFKBIA, CDK6, NFKB1, NFKBIA, PIK3R1, CD4, CD80,
IL10, EGR2, EGR3, NFKB1, NFKBIA, DUSP1, NFKB1, NFKBIA, BIRC2, NFKB1, NFKBIA, TNFSF10, NFKB1,
NFKBIA, TNF, GATA3, IL13, IL5, IL10RA, IL22, JAK2, NFKB1, NFKBIA, TNFRSF9, IL10, IL10RA, TNF, IL7, IL7R,
PIK3R1, CD4, IL12RB1, JAK2, BCL2L1, IKZF3, IL2RA, PIK3R1, CD80, CTLA4, PIK3R1, NFKB1, NFKBIA, PIK3R1,
NFKB1, NR3C1, PIK3R1, BCL2L1, NFKB1, PIK3R1, NFKB1, NFKBIA, TNF, TNFRSF1B, AIM2, ASB2, BCL2L1,
BIRC2, CCR2, CD200R1, CD4, CD79B, CD80, CISH, CSF2, CTLA4, DHX58, EEA1, GBP7, ICAM1, ICAM4, IL1R2,
IL2RA, IL5, IL7, IL7R, IRF4, IRF6, ISG20, ITGAV, JAK2, LNPEP, MAP3K8, MAPKAPK3, NCF4, NFKBIA, NR4A1,
PDCD1, PDCD1LG2, PIK3R1, REL, RIPK3, RPS6KA1, SOCS2, TAP1, TRIM36, CALCA, CCR1, CCR10, CCR2, CCR3,
CCR4, CCR8, CXCL5, CYSLTR1, CYSLTR2, GNG4, GPR55, GPR65, HRH4, NMUR1, PENK, PTGER2, PTGIR,
RAMP1, RAMP3, RLN3, WNT3, XCR1, AR, NR3C1, NR4A1, NR4A2, NR4A3, PPARG, RORA, VDR, CCR1, CCR10,
CCR2, CCR3, CCR4, CCR8, CXCL5, CYSLTR1, CYSLTR2, GPR55, GPR65, HRH4, NMUR1, PENK, PTGER2, PTGIR,
RLN3, XCR1, CCR1, CCR10, CCR2, CCR3, CCR4, CCR8, CXCL5, XCR1, CCR1, CCR10, CCR2, CCR3, CCR4, CCR8,
CXCL5, GNG4, GPR55, HRH4, NMUR1, PENK, RGS9, RLN3, CISH, CSF2, GBP7, ICAM1, IL1R2, IL2RA, IL5, IL7,
IL7R, IRF4, IRF6, ISG20, JAK2, MAP3K8, PIK3R1, SOCS2, CALCA, CAMK4, CCR1, CCR10, CCR2, CCR3, CCR4,
CCR8, CXCL5, CYSLTR1, CYSLTR2, DGKG, GNA14, GNG4, GPR55, GPR65, HRH4, NMUR1, PDE4C, PENK, PIK3R1,
PTGER2, PTGIR, RAMP1, RAMP3, RASGRF1, RGS2, RGS9, RLN3, ROCK1, RPS6KA1, WNT3, XCR1, CALCA,
CCR1, CCR10, CCR2, CCR3, CCR4, CCR8, CXCL5, CYSLTR1, CYSLTR2, DGKG, GNA14, GNG4, GPR55, GPR65,
HRH4, NMUR1, PDE4C, PENK, PIK3R1, PTGER2, PTGIR, RAMP1, RAMP3, RASGRF1, RGS2, RGS9, RLN3, ROCK1,
XCR1, CSF2, IL1R2, IL2RA, IL5, IL7, IL7R, JAK2, MAP3K8, PIK3R1, CSF2, IL2RA, IL5, JAK2, PIK3R1, CD4,
CD80, CTLA4, MAP3K8, PDCD1, PDCD1LG2, PIK3R1, CYSLTR1, CYSLTR2, PTGER2, PTGIR, CCR1, CCR10,
CCR2, CCR3, CCR4, CCR8, CXCL5, NMUR1, PENK, RLN3, XCR1, CSF2, IL2RA, IL5, JAK2, PIK3R1, CYSLTR1,
CYSLTR2, DGKG, GNA14, GNG4, GPR65, NMUR1, PIK3R1, RGS2, RPS6KA1, XCR1, IL7, IL7R, PIK3R1, CSF2, IL2RA,
IL5, JAK2, PIK3R1, ASB2, CD200R1, CD4, CD79B, CD80, CTLA4, ICAM1, ICAM4, ITGAV, LNPEP, MAP3K8, NCF4,
NFKBIA, NR4A1, PDCD1, PDCD1LG2, PIK3R1, REL, TAP1, TRIM36, CALCA, GNG4, PDE4C, PTGER2, PTGIR,
RAMP1, RAMP3, RLN3, CYSLTR1, CYSLTR2, DGKG, GNA14, GNG4, GPR65, NMUR1, PIK3R1, RGS2, XCR1,
BMP7, CCL1, CCR1, CCR10, CCR2, CCR3, CCR4, CCR8, CD70, CSF1, CSF2, CXCL5, IL10, IL10RA, IL12RB1, IL13,
IL1R2, IL22, IL23R, IL2RA, IL5, IL7, IL7R, IL9R, LIF, TNF, TNFRSF13B, TNFRSF18, TNFRSF1B, TNFRSF4, TNFRSF8,
TNFRSF9, TNFSF10, TNFSF11, XCR1, BCL2L1, CISH, CSF2, IL10, IL10RA, IL12RB1, IL13, IL22, IL23R, IL2RA,
IL5, IL7, IL7R, IL9R, JAK2, LIF, PIK3R1, PIM1, SOCS2, CD37, CD4, CSF1, CSF2, IL1R2, IL2RA, IL5, IL7, IL7R,
IL9R, ITGA2, TNF, CCL1, CCR1, CCR10, CCR2, CCR3, CCR4, CCR8, CXCL5, GNG4, GRK4, JAK2, NFKB1, NFKBIA,
PIK3R1, ROCK1, XCR1, CACNA1A, CACNA1S, DUSP1, DUSP14, FGF16, GADD45B, IL1R2, MAP3K8, MAPKAPK3,
NFKB1, NR4A1, PTPN5, PTPN7, RASGRF1, RASGRP4, RELB, RPS6KA1, TNF, CD4, CSF2, CTLA4, IL10,
IL5, MAP3K8, NFKB1, NFKBIA, PDCD1, PIK3R1, TNF, BCL2L1, BIRC2, CDK6, ITGA2, ITGAV, NFKB1, NFKBIA,
PIK3R1, TRAF1, ALCAM, CD4, CD80, CDH15, CTLA4, ICAM1, ITGAV, ITGB8, PDCD1, PDCD1LG2, AR, ARNT2,
BCL2L1, BIRC2, CDK6, EGLN3, FGF16, ITGA2, ITGAV, MMP9, NFKB1, NFKBIA, PIK3R1, PPARG, TRAF1,
WNT3, BCL2L1, BIRC2, NFKB1, NFKBIA, PIK3R1, TNF, TNFSF10, CCR10, CD80, IL10, IL5, TNFRSF13B, IL10, JAK2,
NCF4, NFKB1, NFKBIA, TNF, IL10, IL13, IL5, TNF, CD80, CTLA4, IL10, IL5, TG, AIM2, NFKB1, NFKBIA, RIPK3,
TREX1, CD4, IL7R, TAP1, TNFRSF13B, CD80, IL10, IL5, TNF, JAK2, NFKB1, NFKBIA, TNF, TNFRSF1B

1. A method for activating or differentiating a T cell, the method comprising contacting a T cell with a pharmaceutically effective amount of an agent that inhibits Yap and culturing the T cells for a time, and under conditions sufficient to induce activation or differentiation.

2. The method of claim 1, wherein the contacting is ex vivo, in vivo, or in vitro.

3. The method of claim 1, further comprising the step of, prior to contacting, obtaining a T cell from a biological source.

4. The method of claim 1, wherein the T cell is naïve T cell, a cytotoxic T cell, a memory T cell, a natural killer T cell, a tumor infiltrating T cell, a regulatory T cell, a helper T cell, a synthetic T cell, a αβ T cell, γδ T cell, CD8+ T cell, or a CD4+ T cell.

5. The method of claim 1, wherein the agent that inhibits YAP is selected from the group consisting of a small molecule, a peptide, an antibody, a genome editing system, an antisense oligonucleotide, and an RNAi.

6. The method of claim 5, wherein the small molecule is Verteporfin or YAP/TAZ inhibitor-1.

7. The method of claim 1, wherein inhibiting YAP is inhibiting the expression level and/or activity of YAP in the T cell.

8. The method of claim 7, wherein the expression level and/or activity of YAP is inhibited by at least 50%, at least 60%, at least 70%, at least 80%, at least 90%, or more as compared to an appropriate control.

9. The method of claim 1, wherein inhibiting the expression level and/or activity of YAP in the T cell increases at least one of
the T cell's capacity to infiltrate a tumor microenvironment and/or tumor,
expression of chemokine or chemokine receptor genes in the T cell,
expression of at least one of TEAD1, TEAD2, TEAD3, or TEAD4 protein in the T cell, and
expression of WW domain-containing transcription regulator protein 1 (WWTR1/Taz) in the T cell.

10. The method of claim 1, further comprising contacting the T cell with an agent that inhibits WWTR1/Taz.

11. The method of claim 1, further comprising, aftering cultring, engineering the T cell to comprise a chimeric antigen receptor or genetically modifying the T cell.

12. The method of claim 1, further comprising, after culturing, transplanting said population of contacted T cells into a recipient subject.

13. The method of claim 12, wherein the population of contacted T cells is autologous to the recipient subject, allogeneic to the recipient subject, or xenogeneic to the recipient subject.

14. An activated or differentiated T cell produced by the method of claim 1.

15. A pharmaceutical composition comprising an activated or differentiated T cell of claim 14.

16. The composition of claim 15, formulated for T cell transplantation.

17. A method of treating or preventing a disease or disorder caused by or associated with T cell dysfunction, the method comprising administering to a recipient subject in need thereof a therapeutically effective amount the composition of claim 16 or an agent that inhibits Yap.

18. The method of claim 17, wherein the composition comprises a population of T cells that is autologous to the recipient subject, allogeneic to the recipient subject, or xenogeneic to the recipient subject.

19. The method of claim 17, wherein the disease or disorder caused by or associated with T cell dysfunction is selected from the group consisting of a cancer, an autoimmune disease, and a microbial infectious disease.

20. The method of claim 19, wherein the cancer is a carcinoma, a sarcoma, a melanoma, a lymphoma, and a leukemia.

21. The method of claim 19, wherein the microbial infectious disease is caused by a fungal, bacterial, or viral infection.

22. The method of claim 19, wherein the autoimmune disease is selected from the groups consisting of Lupus, Type I Diabetes, Sjögren's syndrome, Rheumatoid arthritis, Inflammatory bowel disease, Multiple sclerosis, Psoriasis, Guillain-Barre syndrome, Chronic inflammatory demyelinating polyneuropathy, Graves' disease, Hashimoto's thyroiditis, Myasthenia gravis, Vasculitis, Addison's disease, Pernicious anemia, Celiac disease, Systemic lupus erythematosus, Cutaneous lupus erythematosus, and Aplastic anemia.

23. The method of claim 17, further comprising administering an agent that inhibits WWTR1/Taz.

24. The method of claim 17, further comprising administering at least a second therapeutic.


SEQUENCE LISTING (27 sequences)

SEQ ID NO: 1 - DNA, 1515 bp, Homo sapiens
atggatcccgggcagcagccgccgcctcaaccggccccccagggccaagggcagccgccttcgcagcccccgcaggggcagggcccgccgtccggacccgggcaaccggcacccgcggcgacccaggcggcgccgcaggcaccccccgccgggcatcagatcgtgcacgtccgcggggactcggagaccgacctggaggcgctcttcaacgccgtcatgaaccccaagacggccaacgtgccccagaccgtgcccatgaggctccggaagctgcccgactccttcttcaagccgccggagcccaaatcccactcccgacaggccagtactgatgcaggcactgcaggagccctgactccacagcatgttcgagctcattcctctccagcttctctgcagttgggagctgtttctcctgggacactgacccccactggagtagtctctggcccagcagctacacccacagctcagcatcttcgacagtcttcttttgagatacctgatgatgtacctctgccagcaggttgggagatggcaaagacatcttctggtcagagatacttcttaaatcacatcgatcagacaacaacatggcaggaccccaggaaggccatgctgtcccagatgaacgtcacagcccccaccagtccaccagtgcagcagaatatgatgaactcggcttcaggtcctcttcctgatggatgggaacaagccatgactcaggatggagaaatttactatataaaccataagaacaagaccacctcttggctagacccaaggcttgaccctcgttttgccatgaaccagagaatcagtcagagtgctccagtgaaacagccaccacccctggctccccagagcccacagggaggcgtcatgggtggcagcaactccaaccagcagcaacagatgcgactgcagcaactgcagatggagaaggagaggctgcggctgaaacagcaagaactgcttcggcaggcaatgcggaatatcaatcccagcacagcaaattctccaaaatgtcaggagttagccctgcgtagccagttaccaacactggagcaggatggtgggactcaaaatccagtgtcttctcccgggatgtctcaggaattgagaacaatgacgaccaatagctcagatcctttccttaacagtggcacctatcactctcgagatgagagtacagacagtggactaagcatgagcagctacagtgtccctcgaaccccagatgacttcctgaacagtgtggatgagatggatacaggtgatactatcaaccaaagcaccctgccctcacagcagaaccgtttcccagactaccttgaagccattcctgggacaaatgtggaccttggaacactggaaggagatggaatgaacatagaaggagaggagctgatgccaagtctgcaggaagctttgagttctgacatccttaatgacatggagtctgttttggctgccaccaagctagataaagaaagctttcttacatggttatag

SEQ ID NO: 2 - protein, 504 aa, Homo sapiens
MDPGQQPPPQPAPQGQGQPPSQPPQGQGPPSGPGQPAPAATQAAPQAPPAGHQIVHVRGDSETDLEALFNAVMNPKTANVPQTVPMRLRKLPDSFFKPPEPKSHSRQASTDAGTAGALTPQHVRAHSSPASLQLGAVSPGTLTPTGVVSGPAATPTAQHLRQSSFEIPDDVPLPAGWEMAKTSSGQRYFLNHIDQTTTWQDPRKAMLSQMNVTAPTSPPVQQNMMNSASGPLPDGWEQAMTQDGEIYYINHKNKTTSWLDPRLDPRFAMNQRISQSAPVKQPPPLAPQSPQGGVMGGSNSNQQQQMRLQQLQMEKERLRLKQQELLRQAMRNINPSTANSPKCQELALRSQLPTLEQDGGTQNPVSSPGMSQELRTMTTNSSDPFLNSGTYHSRDESTDSGLSMSSYSVPRTPDDFLNSVDEMDTGDTINQSTLPSQQNRFPDYLEAIPGTNVDLGTLEGDGMNIEGEELMPSLQEALSSDILNDMESVLAATKLDKESFLTWL

SEQ ID NO: 3 - DNA, 1203 bp, Homo sapiens
atgaatccggcctcggcgccccctccgctcccgccgcctgggcagcaagtgatccacgtcacgcaggacctagacacagacctcgaagccctcttcaactctgtcatgaatccgaagcctagctcgtggcggaagaagatcctgccggagtctttctttaaggagcctgattcgggctcgcactcgcgccagtccagcaccgactcgtcgggcggccacccggggcctcgactggctgggggtgcccagcatgtccgctcgcactcgtcgcccgcgtccctgcagctgggcaccggcgcgggtgctgcgggtagccccgcgcagcagcacgcgcacctccgccagcagtcctacgacgtgaccgacgagctgccactgcccccgggctgggagatgaccttcacggccactggccagaggtacttcctcaatcacatagaaaaaatcaccacatggcaagaccctaggaaggcgatgaatcagcctctgaatcatatgaacctccaccctgccgtcagttccacaccagtgcctcagaggtccatggcagtatcccagccaaatctcgtgatgaatcaccaacaccagcagcagatggcccccagtaccctgagccagcagaaccaccccactcagaacccacccgcagggctcatgagtatgcccaatgcgctgaccactcagcagcagcagcagcagaaactgcggcttcagagaatccagatggagagagaaaggattcgaatgcgccaagaggagctcatgaggcaggaagctgccctctgtcgacagctccccatggaagctgagactcttgccccagttcaggctgctgtcaacccacccacgatgaccccagacatgagatccatcactaataatagctcagatcctttcctcaatggagggccatatcattcgagggagcagagcactgacagtggcctggggttagggtgctacagtgtccccacaactccggaggacttcctcagcaatgtggatgagatggatacaggagaaaacgcaggacaaacacccatgaacatcaatccccaacagacccgtttccctgatttccttgactgtcttccaggaacaaacgttgacttaggaactttggaatctgaagacctgatccccctcttcaatgatgtagagtctgctctgaacaaaagtgagccctttctaacctggctgtaa

SEQ ID NO: 4 - protein, 400 aa, Homo sapiens
MNPASAPPPLPPPGQQVIHVTQDLDTDLEALFNSVMNPKPSSWRKKILPESFFKEPDSGSHSRQSSTDSSGGHPGPRLAGGAQHVRSHSSPASLQLGTGAGAAGSPAQQHAHLRQQSYDVTDELPLPPGWEMTFTATGQRYFLNHIEKITTWQDPRKAMNQPLNHMNLHPAVSSTPVPQRSMAVSQPNLVMNHQHQQQMAPSTLSQQNHPTQNPPAGLMSMPNALTTQQQQQQKLRLQRIQMERERIRMRQEELMRQEAALCRQLPMEAETLAPVQAAVNPPTMTPDMRSITNNSSDPFLNGGPYHSREQSTDSGLGLGCYSVPTTPEDFLSNVDEMDTGENAGQTPMNINPQQTRFPDFLDCLPGTNVDLGTLESEDLIPLFNDVESALNKSEPFLTWL

SEQ ID NO: 5 - DNA, 20 bp, Artificial Sequence
  Description of Artificial Sequence Synthetic oligonucleotide
catcagatcgtgcacgtccg

SEQ ID NO: 6 - DNA, 20 bp, Artificial Sequence
  Description of Artificial Sequence Synthetic oligonucleotide
gctgcgaaggcggctgccct

SEQ ID NO: 7 - DNA, 20 bp, Artificial Sequence
  Description of Artificial Sequence Synthetic oligonucleotide
cagggcccgccgtccggacc

SEQ ID NO: 8 - DNA, 19 bp, Artificial Sequence
  Description of Artificial Sequence Synthetic oligonucleotide
tgccccagaccgtgcccat

SEQ ID NO: 9 - DNA, 20 bp, Artificial Sequence
  Description of Artificial Sequence Synthetic oligonucleotide
atccgaagcctagctcgtgg

SEQ ID NO: 10 - DNA, 20 bp, Artificial Sequence
  Description of Artificial Sequence Synthetic oligonucleotide
tgtctaggtcctgcgtgacg

SEQ ID NO: 11 - DNA, 20 bp, Artificial Sequence
  Description of Artificial Sequence Synthetic oligonucleotide
ctctgctccctcgaatgata

SEQ ID NO: 12 - DNA, 20 bp, Artificial Sequence
  Description of Artificial Sequence Synthetic oligonucleotide
tctcatgtctggggtcatcg

SEQ ID NO: 13 - RNA, 19 nt, Artificial Sequence
  Description of Artificial Sequence Synthetic oligonucleotide
uguggaugagauggauaca

SEQ ID NO: 14 - RNA, 19 nt, Artificial Sequence
  Description of Artificial Sequence Synthetic oligonucleotide
uguauccaucucauccaca

SEQ ID NO: 15 - RNA, 19 nt, Artificial Sequence
  Description of Artificial Sequence Synthetic oligonucleotide
gacaucuucuggucagaga

SEQ ID NO: 16 - RNA, 19 nt, Artificial Sequence
  Description of Artificial Sequence Synthetic oligonucleotide
ucucugaccagaagauguc

SEQ ID NO: 17 - RNA, 19 nt, Artificial Sequence
  Description of Artificial Sequence Synthetic oligonucleotide
ggugauacuaucaaccaaa

SEQ ID NO: 18 - RNA, 19 nt, Artificial Sequence
  Description of Artificial Sequence Synthetic oligonucleotide
uuugguugauaguaucacc

SEQ ID NO: 19 - RNA, 19 nt, Artificial Sequence
  Description of Artificial Sequence Synthetic oligonucleotide
caauaugaauauggagauc

SEQ ID NO: 20 - RNA, 19 nt, Artificial Sequence
  Description of Artificial Sequence Synthetic oligonucleotide
gaucuccauauucauauug

SEQ ID NO: 21 - RNA, 18 nt, Artificial Sequence
  Description of Artificial Sequence Synthetic oligonucleotide
acguugacuuaggaacuu

SEQ ID NO: 22 - RNA, 18 nt, Artificial Sequence
  Description of Artificial Sequence Synthetic oligonucleotide
aaguuccuaagucaacgu

SEQ ID NO: 23 - RNA, 19 nt, Artificial Sequence
  Description of Artificial Sequence Synthetic oligonucleotide
agguacuuccucaaucaca

SEQ ID NO: 24 - RNA, 19 nt, Artificial Sequence
  Description of Artificial Sequence Synthetic oligonucleotide
ugugauugaggaaguaccu

SEQ ID NO: 25 - RNA, 19 nt, Artificial Sequence
  Description of Artificial Sequence Synthetic oligonucleotide
gguacuuccucaaucacau

SEQ ID NO: 26 - RNA, 19 nt, Artificial Sequence
  Description of Artificial Sequence Synthetic oligonucleotide
augugauugaggaaguacc

SEQ ID NO: 27 - protein, 4 aa, Artificial Sequence
  Description of Artificial Sequence Synthetic peptide
RGDS